(19) United States
(12) Patent Application Publication
(10) Pub. No.: US 2026/0020841 A1
(43) Pub. Date: Jan. 22, 2026

(54) VESSEL CONTOUR TRACING AND BLOOD FLOW DETECTION IN INTRAVASCULAR ULTRASOUND IMAGING AND ASSOCIATED DEVICES, SYSTEMS, AND METHODS

(71) Applicant: Inari Medical, Inc., Irvine, CA (US)

(72) Inventors: Randall D. Hamlin, Irvine, CA (US); Haibo Wang, Cambridge, MA (US); Grzegorz Toporek, Cambridge, MA (US); Tong Xiao, Cambridge, MA (US); Sezen Yagmur Onol, Cambridge, MA (US)

(21) Appl. No.: 19/267,931

(22) Filed: Jul. 14, 2025

Related U.S. Application Data
(60) Provisional application No. 63/672,654, filed on Jul. 17, 2024.
(60) Provisional application No. 63/692,964, filed on Sep. 10, 2024.

(51) Int. Cl. A61B 8/12; A61B 8/00; A61B 8/06

(52) U.S. Cl. CPC A61B 8/12; A61B 8/06; A61B 8/465

(57) ABSTRACT

Disclosed herein is intravascular ultrasound (“IVUS”) system is disclosed which includes an IVUS console for generating a vessel contour trace and a blood flow image from an IVUS image. The IVUS console includes a communication interface; a memory circuit; an image output display; and a processor circuit configured to, when an interim vessel contour trace meets or exceeds accuracy criteria, display on the image output display a complete vessel contour trace comprising the interim vessel contour trace with user-interactable key nodes or control points and merge or superimpose a blood flow IVUS image with the complete vessel contour trace.

### CROSS-REFERENCE TO RELATED APPLICATION

This application claims the benefit of (i) U.S. Provisional Patent Application No. 63/672,654, filed Jul. 17, 2024, and titled “APPARATUS, METHOD AND SYSTEM FOR AUTOMATIC AND USER-INTERACTIVE VESSEL CONTOUR TRACING IN INTRAVASCULAR ULTRASOUND IMAGING,” and (ii) U.S. Provisional Patent Application No. 63/692,964, filed Sep. 10, 2024, and titled “APPARATUS, METHOD AND SYSTEM FOR AUTOMATIC AND USER-INTERACTIVE BLOOD FLOW DETECTION AND IMAGING IN INTRAVASCULAR ULTRASOUND,” which are incorporated herein by reference in their entirety.

### TECHNICAL FIELD

The present technology generally relates to intravascular ultrasound imaging, and more particularly, relates to an apparatus, method, and system for vessel contour tracing and blood flow detection in intravascular ultrasound imaging.

### BACKGROUND

Intravascular ultrasound (“IVUS”) imaging is used in interventional cardiology as a diagnostic tool for a diseased vessel within the human body, such as an artery or vein, to determine the need for treatment, to guide the intervention, and/or to assess its effectiveness. Example systems may include imaging catheters that may be used within the vascular system to transmit sound waves with the purpose of creating two-dimensional (2D) images from inside the vasculature. An IVUS catheter, may include one or more ultrasound transducers (also referred to equivalently herein as ultrasonic transducers) that emit and receive ultrasonic energy, and may be inserted into the vessel of interest and guided to the region to be imaged. The ultrasonic waves are reflected, to varying degrees and at varying depths, by discontinuities in the tissue structure and density, blood cells, and other anatomical and physiological features. The reflected ultrasonic waves are, in turn, received by one or more of the ultrasound transducers and converted into electrical signals, which, in turn, are provided to an IVUS imaging system. The IVUS imaging system processes the received signals to produce an image, such as a cross-sectional image of the vasculature in the region where the IVUS has been placed. These images are then used to determine the vessel anatomy, the existence or extent of disease, such as clot or other obstruction formation, and to measure the dimensions of the vessel area and degree of stenosis, for example, and without limitation.

During such vascular imaging, it is often imperative to obtain an accurate contour trace of the vessel wall for several reasons. First, an accurate contour trace will help determine the effective diameter of the vessel, which is highly significant for determining the sizing of a vascular stent to be inserted into the vessel. An improperly sized vascular stent can be highly detrimental to the subject or patient, resulting in significant pain if the selected size is too large for the vessel or potential dislodging and displacement if the selected size is too small. Second, an accurate contour trace may help determine the percent of vessel occlusion, if any, which in turn will typically impact medical and/or surgical treatment decisions. Tracing a venous wall boundary may be clinically invaluable, but current methods are both time-consuming and irreproducible.

Vessel wall (lumen/border) detection systems for intravascular ultrasound imaging of deep veins that are both accurate and robust to artifacts may be advantageous. Ultrasound images, especially IVUS image data, are relatively noisy, and measurement interobserver variability may be high. Currently, veins may be traced manually by end-users. This is often very time-consuming and requires several years of experience to gain accuracy. Intra- and interobserver variability is also very high. For instance, most of the users may inaccurately place the trace around a “ringdown” artifact surrounding the catheter and overlapping into the actual vessel wall, whereas the catheter itself may be touching the vessel wall and the trace may be provided between the catheter and the wall.

Conventional image processing techniques, such as edge detection or ellipse fitting, may also be applied to vessel wall tracing. These approaches tend to be highly limited, such as to images that have a clear vein edge, have an elliptical shape, and lack strong ultrasound artifacts (such as acoustic shadowing, reverberation, and grating lobes). In practice, the venous wall edge is often unclear due to low IVUS quality, imaging artifacts, and the presence of clots and arteries. Moreover, the vein shape may not be elliptical, due to compression or blood clots. Ringdown and blood flow can also bring challenges to vein tracing.

Additionally, during such vascular imaging, it is often significant and imperative to obtain accurate images, detections, and/or assessments of venous blood flow, for several reasons. First, an accurate image, detection, and/or assessment of venous blood flow may help determine the percent of vessel occlusion, if any, which in turn may impact medical and/or surgical treatment decisions, such as performance of a thrombectomy, for example. Second, an accurate image, detection, and/or assessment of venous blood flow may help determine the presence or absence of any thrombus (blood clot), and differentiate such a thrombus from other kinds of slow or turbulent flow (typically referred to as “smoke” in ultrasound images). Third, an accurate image, detection, and/or assessment of venous blood flow may help determine the effective diameter of the vessel, which is highly significant for determining the sizing of a vascular stent to be inserted into the vessel. An improperly sized vascular stent can be highly detrimental to the subject or patient, resulting in significant pain if the selected size is too large for the vessel or potential dislodging and displacement if the selected size is too small. Fourth, an accurate image, detection, and/or assessment of venous blood flow may be utilized to differentiate a thrombus from other tissue or imaging artifacts.

Currently, blood flow imaging may not be readily available using intravascular ultrasound catheterization procedures. Instead, blood flow imaging may be performed using a hand-held, considerably larger transducer array or system that is external to the patient, typically held against the patient's skin in the vicinity of the region of interest. The standard techniques may utilize Doppler flow imaging methods, which measure the frequency shift between the transmitted and reflected signals, and can detect blood flow magnitude and direction. Such Doppler imaging, however, has not been incorporated into any IVUS imaging. The key limitation of a Doppler technique is its requirement to transmit ultrasound beams within a range of transmit angles that are largely unavailable in IVUS systems. For example, to obtain proper reflected signals from flowing blood, Doppler imaging typically requires emitting the ultrasound beams or signals generally along the same direction as the motion to be detected (e.g., between 30° to 60° from the direction of flow motion). These required transmit angles are typically unavailable in IVUS systems, in which the ultrasonic transducers are located on the elongated catheter body and the emitted ultrasound signals are typically perpendicular (90°) to the elongated catheter body, the vessel walls, and the direction of blood flow.

Other approaches to blood flow imaging have utilized various filter banks, in which multiple filters are tuned for capturing different levels of motion. Such prior methods, however, have significant computational time delays, and furthermore, do not have sufficient sensitivity to distinguish thrombus formation from slow or turbulent blood flow. Many of these methods also operate under the assumption that the catheter or probe is arranged in the center of the vessel, which is typically not the case and, instead, the catheter or probe is often touching the vessel wall.

### BRIEF DESCRIPTION OF THE DRAWINGS

Many aspects of the present technology can be better understood with reference to the following drawings. The components in the drawings are not necessarily to scale. Instead, emphasis is placed on illustrating clearly the principles of the present disclosure.

FIG. 1 is an electrical block diagram illustrating a representative embodiment of an intravascular ultrasound (“IVUS”) system in accordance with embodiments of the present technology.

FIG. 2 is an electrical block diagram illustrating a representative embodiment of an IVUS (host) console in accordance with embodiments of the present technology.

FIG. 3 is an electrical block diagram illustrating a representative embodiment of a patient interface ultrasound module (“PIUM”) in accordance with embodiments of the present technology.

FIG. 4 is a first electrical block diagram illustrating a representative embodiment of an IVUS catheter in accordance with embodiments of the present technology.

FIG. 5 is a second, more detailed electrical block diagram illustrating a representative embodiment of an IVUS catheter in accordance with embodiments of the present technology.

FIGS. 6A and 6B are isometric views illustrating a representative embodiment of an IVUS catheter 200 in accordance with embodiments of the present technology.

FIG. 7 is a cross-sectional view (through the A-A′ plane of FIG. 6A) illustrating a representative circumferential ultrasonic transducer array of the representative embodiment of the IVUS catheter in accordance with embodiments of the present technology.

FIG. 8 is a diagram illustrating a frame or matrix of digital ultrasound signal data utilized to form an ultrasound image using the IVUS catheter in accordance with embodiments of the present technology.

FIG. 9 is a photograph illustrating a first ultrasound image of a vein and illustrating an overlaid, representative first user-interactive vessel contour trace in accordance with embodiments of the present technology.

FIG. 10 is a photograph illustrating a second ultrasound image of a vein and illustrating a ringdown artifact.

FIG. 11 is a photograph illustrating the second ultrasound image of a vein of FIG. 10 and illustrating an overlaid, representative second user-interactive vessel contour trace in accordance with embodiments of the present technology.

FIG. 12 is a photograph illustrating the second ultrasound image of a vein of FIG. 10 and illustrating an overlaid manual vessel contour trace together with an overlaid, representative third user-interactive vessel contour trace in accordance with embodiments of the present technology.

FIG. 13 is a photograph illustrating the second ultrasound image of a vein of FIG. 10 and illustrating an overlaid fourth user-interactive vessel contour trace together with an overlaid, representative fifth user-interactive vessel contour trace in accordance with embodiments of the present technology.

FIGS. 14A and 14B are flow diagrams illustrating a computing system implemented method to generate and display a user-interactive vessel contour trace in an ultrasound image provided using the IVUS catheter in accordance with embodiments of the present technology.

FIG. 15 is a flow diagram illustrating a computing system implemented method to remove a ringdown artifact in an ultrasound image provided using the IVUS catheter in accordance with embodiments of the present technology.

FIGS. 16A and 16B are flow diagrams illustrating a computing system implemented segmentation method to generate an initial vessel contour trace in accordance with embodiments of the present technology.

FIG. 17 is a diagram illustrating a frame or matrix of digital ultrasound signal data (RF line data) which has been padded with additional data in accordance with embodiments of the present technology.

FIG. 18 is a diagram illustrating a RF line data which has been rotated for double-inference during segmentation in accordance with embodiments of the present technology.

FIG. 19 is a flow diagram illustrating a computing system implemented method for active contouring and statistical shape modelling to generate and display the user-interactive vessel contour trace in an ultrasound image provided using the IVUS catheter in accordance with embodiments of the present technology.

FIGS. 20A and 20B are flow diagrams illustrating a computing system implemented method for statistical shape modelling to generate and display the user-interactive vessel contour trace in an ultrasound image provided using the IVUS catheter in accordance with embodiments of the present technology.

FIG. 21 is a first ultrasound image of a vein with a ringdown artifact, in accordance with embodiments of the present technology.

FIG. 22 is a second ultrasound image illustrating the first ultrasound image of FIG. 21, and further illustrating an overlaid, representative image of blood flow in accordance with embodiments of the present technology.

FIGS. 23A, 23B, and 23C are flow diagrams illustrating a computing system implemented method to generate and display a user-interactive blood flow image in an ultrasound image provided using the IVUS catheter in accordance with embodiments of the present technology.

FIG. 24 is a first ultrasound RF line data frame, utilized as a reference frame, illustrating selection of a first subframe (or block) for block or subframe searching and/or matching in accordance with embodiments of the present technology.

FIG. 25 is a second (or next) ultrasound RF line data frame illustrating selection of a second subframe (or block) for block or subframe searching and/or matching in accordance with embodiments of the present technology.

FIG. 26 is a diagram illustrating block or subframe searching and/or matching of the first subframe (or block) within the second subframe (or block) in accordance with embodiments of the present technology.

FIGS. 27A, 27B, 27C, and 27D are a sequence of diagrams illustrating block or subframe searching and/or matching of the first subframe (or block) within a sliding window of the second subframe (or block) in accordance with embodiments of the present technology.

FIG. 28 is a diagram illustrating selection of a region matching the first subframe (or block) within the second subframe (or block) and distance measurement for block or subframe searching and/or matching in accordance with embodiments of the present technology.

FIG. 29 is an IVUS console display screen shot or image illustrating a graphical user interface having a fourth ultrasound image of a vein and illustrating an overlaid, representative user-interactive blood flow detection and color selection interface in accordance with the disclosure of the disclosure herein.

### DETAILED DESCRIPTION

### I. Introduction

The present technology is generally directed to an apparatus, method, and system for vessel contour tracing in intravascular ultrasound imaging. The representative intravascular ultrasound (“IVUS”) apparatus, method, and system embodiments provide for assisting the end-user to obtain accurate measurements of certain biomarkers, such as vein/vessel area, minimum diameter, maximum diameter, average diameter and effective diameter, for example. Such an IVUS apparatus, method, and system uses a single or a plurality of IVUS images or a short IVUS sequence as input, and on each IVUS input, the representative IVUS apparatus, method, and system embodiments trace the vein wall and display the traced vein shape to the end-user (provided the vessel contour trace is considered sufficiently accurate). The representative IVUS apparatus, method, and system embodiments allow the user to accept the traced vein shape, edit it manually, or ignore it and re-trace the vein manually. Once the auto-tracing is completed, the representative IVUS apparatus, method, and system embodiments are able to calculate the biomarker measurements, among other features. The representative IVUS apparatus, method, and system embodiments provide reliable and reproducible vessel contour tracing, including avoiding various or typical user mistakes, such as the erroneous inclusion of the ringdown artifact within the region of the vessel contour trace.

A representative embodiment of a computing system implemented method of generating a vessel contour trace from an IVUS image is disclosed, with the computing system including an IVUS console having a processor circuit and an image output display.

A representative embodiment of an IVUS console for generating a vessel contour trace from an IVUS image is also disclosed, with the IVUS console comprising: a communication interface to receive a first frame of RF line data for the IVUS image and receive a user selection for the generation of the vessel contour trace; a memory circuit configured to store training data; an image output display; and a processor circuit coupled to the communication interface, to the memory circuit and to the image output display, the processor circuit configured to: convert the first frame of RF line data to Cartesian coordinates and display the IVUS image on the image output display; generate a segmentation of the IVUS image to identify the vessel of interest for the vessel contour trace; generate an initial vessel contour trace from the generated segmentation; generate an interim vessel contour trace using an active contour model initialized with the initial vessel contour trace and constrained with a statistical vein shape model (“SSM”); and the processor circuit configured, when the interim vessel contour trace meets or exceeds accuracy criteria, to display on the image output display a complete vessel contour trace comprising the interim vessel contour trace with user-interactable key nodes or control points.

In some embodiments, the processor circuit is further configured to generate and displaying on the image output display one or more selected biometrics. In some embodiments, the processor circuit is further configured to remove any ringdown artifact from the ultrasound image. In some embodiments, the processor circuit is further configured, when a user has selected autocorrection, to use a user-provided manual trace as the initial vessel contour trace and to display the user-provided manual trace on the image output display. In some embodiments, the processor circuit is further configured, when a user has selected autocompletion, to use user-provided key nodes as accurate or true key nodes in the complete vessel contour trace. In some embodiments, the processor circuit is further configured to convert the initial vessel contour trace, from RF line data, into a Cartesian space coordinate system.

In some embodiments, the processor circuit is further configured to modify the vessel contour trace in the region between an IVUS catheter and a vessel wall.

In some embodiments, the processor circuit is further configured, when the interim vessel contour trace does not meet or exceed the accuracy criteria, to generate a prompt to a user to select or input one or more key nodes or other control points. In some embodiments, the processor circuit is further configured, when a user modifies the generated vessel contour trace, to generate a next vessel contour trace incorporating the user modifications.

In some embodiments, the processor circuit is further configured to generate the segmentation by: generating a plurality of RF line data channels; combining the plurality of RF line data channels to generate a combined RF line data channel; generating a second frame of RF line data having an orientation shift from the first frame of RF line data; obtaining a first segmentation prediction result using the first frame of RF line data; obtaining a second segmentation prediction result using the second frame of RF line data; rotating the second segmentation prediction result to remove the orientation shift; combining or merging the first segmentation prediction result with the orientation-shifted second segmentation prediction result to generate a combined segmentation prediction result; and extracting edge data from the combined segmentation prediction result to generate the initial vessel contour trace.

In some embodiments, the processor circuit is further configured to generate the plurality of RF line data channels by: creating a first, positionally weighted RF line data channel; creating a second, depth normalization RF line data channel; and using the first frame of RF line data as a third RF line data channel. In some embodiments, the processor circuit is further configured to provide data padding to the combined RF line data channel. In some embodiments, the processor circuit is further configured to de-pad the combined segmentation prediction result and re-size the IVUS image. In some embodiments, wherein the processor circuit is further configured to filter the combined segmentation prediction result to retain the largest or maximum segmentation indicative of the vessel of interest.

In some embodiments, the processor circuit is further configured to generate the interim vessel contour trace by iteratively moving the initial vessel contour trace toward the vessel wall edge by optimizing an energy function. In some embodiments, the energy function comprises a plurality of parameters or constraints, the plurality of parameters or constraints comprising a statistical shape energy or constraint; an edge-based energy or constraint; and a region-based energy or constraint. In some embodiments, the edge-based energy constraint utilizes the intensity gradient in the image to move the initial vessel contour trace toward the highest gradient indicative of the edge of the vessel wall. In some embodiments, the region-based energy constraint maximizes the contrast between the intensity of the data averaged within the initial vessel contour trace, and the intensity of the data averaged over an elliptical shell.

In some embodiments, the processor circuit is further configured to generate the interim vessel contour trace by fitting a variation of an ellipse to the initial vessel contour trace using the SSM. In some embodiments, the processor circuit is further configured to modify the interim vessel contour trace to account for a contour of an edge of the vessel wall in the vicinity of a ringdown artifact.

In some embodiments, the processor circuit is further configured to train the SSM model by: sampling a fixed number of points from each shape or contour of a training data set and geometrically align and scaling the training data; computing a mean shape or contour “x̅”; and extracting the main modes of shape or contour variation by calculating the eigenvalues of the covariance matrix (Cov=M·M^{T}) of all of the training data to generate a matrix of eigenvalues (λ₁, λ₂, λ₃, λ₄, . . . λ_{N}), a corresponding matrix of variations (e₁, e₂, e₃, e₄, . . . e_{N}, with each “e” being a vector), and corresponding weightings (w₁, w₂, w₃, w₄, . . . w_{N}).

In some embodiments, the SSM model may be represented as a mean with weighted (“wᵢ”) variations:

$$x = \overline{x} + {\sum }_{i = 0}^{n} {w}_{i} {e}_{i} .$$

In some embodiments, the processor circuit is further configured to fit the initial or interim vessel contour trace to the SSM model by: sampling and scaling points of the initial or interim vessel contour trace to match the coordinate space of the mean shape; aligning the scaled initial or interim vessel contour trace with the mean shape (x̅); applying the SSM model to the aligned shape (“x”) to obtain the weightings:

$${w}_{i} = {{e}_{i} ( x - \overline{x} )}^{T} .$$

In some embodiments, the processor circuit is further configured to determine that the interim vessel contour trace meets or exceeds accuracy criteria when the weightings wi for the initial or interim vessel contour trace are within a predetermined amount or level of the corresponding eigenvalues (λ₁, λ₂, λ₃, λ₄, . . . ).

In some embodiments, the processor circuit is further configured to determine that the interim vessel contour trace meets or exceeds accuracy criteria when:

|wᵢ|≤\sqrt{3}|λi| (with ∥ indicating an absolute value).

A representative embodiment of a computing system implemented method of generating a vessel contour trace from an IVUS image is disclosed, with the computing system including an IVUS console having a processor circuit and an image output display, with the method comprising: using the IVUS console, receiving a first frame of RF line data for the IVUS image; using the processor circuit of the IVUS console, converting the first frame of RF line data to Cartesian coordinates and displaying the IVUS image; using the IVUS console, receiving a user selection for the generation of the vessel contour trace; using the processor circuit of the IVUS console, generating a segmentation of the IVUS image to identify the vessel of interest for the vessel contour trace; using the processor circuit of the IVUS console, generating an initial vessel contour trace from the generated segmentation; using the processor circuit of the IVUS console, generating an interim vessel contour trace using an active contour model initialized with the initial vessel contour trace and constrained with a SSM; and using the processor circuit of the IVUS console, when the interim vessel contour trace meets or exceeds accuracy criteria, displaying on the image output display a complete vessel contour trace comprising the interim vessel contour trace with user-interactable key nodes or control points.

In some embodiments, the computing system implemented method further comprises: using the processor circuit of the IVUS console, generating and displaying on the image output display one or more selected biometrics. In some embodiments, the computing system implemented method further comprises: using the IVUS console and removing any ringdown artifact from the ultrasound image.

In some embodiments, the step of receiving a user selection for the generation of the vessel contour trace further comprises: using the processor circuit of the IVUS console, when a user has selected autocorrection, using a user-provided manual trace as the initial vessel contour trace and displaying the user-provided manual trace on the image output display. In some embodiments, the step of receiving a user selection for the generation of the vessel contour trace further comprises: using the processor circuit of the IVUS console, when a user has selected autocompletion, using user-provided key nodes as accurate or true key nodes in the complete vessel contour trace.

In some embodiments, the computing system implemented method further comprises: using the processor circuit of the IVUS console, converting the initial vessel contour trace, from RF line data, into a Cartesian space coordinate system. In some embodiments, the computing system implemented method further comprises: using the processor circuit of the IVUS console, modifying the vessel contour trace in the region between an IVUS catheter and a vessel wall. In some embodiments, the computing system implemented method further comprises: using the processor circuit of the IVUS console, when the interim vessel contour trace does not meet or exceed the accuracy criteria, generating a prompt to a user to select or input one or more key nodes or other control points.

In some embodiments, the computing system implemented method further comprises: using the processor circuit of the IVUS console, when a user modifies the generated vessel contour trace, generating a next vessel contour trace incorporating the user modifications.

In some embodiments, the step of generating the segmentation further comprises: using the processor circuit of the IVUS console, generating a plurality of RF line data channels; using the processor circuit of the IVUS console, combining the plurality of RF line data channels to generate a combined RF line data channel; using the processor circuit of the IVUS console, generating a second frame of RF line data having an orientation shift from the first frame of RF line data; using the processor circuit of the IVUS console, and using the first frame of RF line data, obtaining a first segmentation prediction result; using the processor circuit of the IVUS console, and using the second frame of RF line data, obtaining a second segmentation prediction result; using the processor circuit of the IVUS console, rotating the second segmentation prediction result to remove the orientation shift; using the processor circuit of the IVUS console, combining or merging the first segmentation prediction result with the orientation-shifted second segmentation prediction result to generate a combined segmentation prediction result; and using the processor circuit of the IVUS console, extracting edge data from the combined segmentation prediction result to generate the initial vessel contour trace.

In some embodiments, the step of generating the plurality of RF line data channels further comprises: using the processor circuit of the IVUS console, creating a first, positionally weighted RF line data channel; using the processor circuit of the IVUS console, creating a second, depth normalization RF line data channel; and using the processor circuit of the IVUS console, using the first frame of RF line data as a third RF line data channel.

In some embodiments, the computing system implemented method further comprises: using the processor circuit of the IVUS console, providing data padding to the combined RF line data channel. In some embodiments, the computing system implemented method further comprises: using the processor circuit of the IVUS console, de-padding the combined segmentation prediction result and re-sizing the IVUS image. In some embodiments, the computing system implemented method further comprises: using the processor circuit of the IVUS console, filtering the combined segmentation prediction result to retain the largest or maximum segmentation indicative of the vessel of interest.

Representative apparatus, method and system embodiments provide imaging, detection, and/or assessment of venous blood flow in intravascular ultrasound imaging. The representative IVUS apparatus, method, and system embodiments provide for an accurate image, detection, and/or assessment of venous blood flow to help determine the percent of vessel occlusion, if any, which in turn will typically impact medical and/or surgical treatment decisions, such as performance of a thrombectomy, for example. The representative IVUS apparatus, method, and system embodiments provide for an accurate image, detection, and/or assessment of venous blood flow to help determine the presence or absence of any thrombus (blood clot), and differentiate such a thrombus from other kinds of slow or turbulent flow (e.g., “smoke”). The representative IVUS apparatus, method, and system embodiments provide for an accurate image, detection, and/or assessment of venous blood flow to help determine the effective diameter of the vessel, which is highly significant for determining the sizing of a vascular stent to be inserted into the vessel. The representative IVUS apparatus, method, and system embodiments provide for an accurate image, detection, and/or assessment of venous blood flow to aid differentiation of a thrombus from other tissue or imaging artifacts.

A representative embodiment of a computing system implemented method for detecting blood flow and generating a blood flow IVUS image is disclosed, with the computing system including an IVUS console having a processor circuit and an image output display.

In some embodiments, multiple consecutive ultrasonic waves are triggered at each location for capturing the subtle blood cell displacements and ensemble of the reflected ultrasonic waves are provided into at least two compounding sections: brightness mode and turbulence. The brightness mode is designed to enhance the stability of the obtained image to eliminate noise interference; whereas the turbulence stresses the difference between each ensemble both in magnitude and angle domains.

A representative embodiment of an IVUS console for detecting blood flow and generating a blood flow IVUS image is also disclosed, with the IVUS console comprising: a communication interface configured to receive a plurality of frames of RF line data for the blood flow IVUS image, the plurality of frames of RF line data comprising a first frame of RF line data and a second frame of RF line data; a memory circuit; an image output display; and a processor circuit coupled to the communication interface, to the memory circuit and to the image output display, the processor circuit configured to: select a subframe of the first RF line data frame; select a larger subframe of the second RF line data frame where the coordinates of the first subframe are included in the second subframe; perform block matching to calculate the similarity score between the first subframe and each subregions in the second subframe for detecting the best displacement position; convert displacement positions into a color image or mask to displace as as visual image of the blood flow; generate a brightness-mode (“B-mode”) IVUS image from one or more frames of RF line data of the plurality of frames of RF line data; and merge or superimpose the color image or mask on or with the B-mode IVUS image to generate the blood flow IVUS image to display on the image output display.

In some embodiments, the block matching comprises a comparison of at least one first pattern of a plurality of speckle pixels within the selected first subframe with at least one second pattern of a plurality of speckle pixels within the selected second subframe.

In some embodiments, a binary mask is configured to eliminate the contamination of tissue clutter, noise and other imaging artifacts from the block matching search to reduce computational time. The binary mask is a weighted composition of, but not limited to, pixel intensity thresholding, locational confidence metric and turbulence subregional criteria that focuses on motion. The mask is utilized as a soft criteria to the first and second RF line data frames.

In some embodiments, the communication interface is further configured to receive a user selection of one or more signal acquisition modes. In some embodiments, the processor circuit is further configured to determine one or more signal acquisition modes. In some embodiments, the communication interface is further configured to receive a user selection of blood flow detection and color parameters. In some embodiments, the processor circuit is further configured to apply the user-selected blood flow detection and color parameters.

In some embodiments, the processor circuit is further configured to apply one or more thresholds to RF line data of the first or second frames of RF line data. In some embodiments, the processor circuit is further configured to remove any ringdown artifact from the first and second RF line data frames. In some embodiments, the processor circuit is further configured to perform clutter filtering of the first and second RF line data frames. In some embodiments, the processor circuit is further configured to provide data padding to the second RF line data frame. In some embodiments, the processor circuit is further configured to apply positional (or resolutional) weighting to the first and second RF line data frames.

In some embodiments, the processor circuit is further configured to limit a search region of the second RF line data frame to a region bounded by a vessel contour trace. In some embodiments, the processor circuit is further configured to separate the first and second RF line data frames into a plurality of separate search regions.

In some embodiments, the processor circuit is further configured to determine one or more blood flow metrics, the one or more blood flow metrics comprising a displacement determination and unpadding operation to the velocity mask to match the b-mode imaging size.

In some embodiments, the processor circuit is further configured to apply spatial and temporal smoothing to the third RF line data frame. In some embodiments, the processor circuit is further configured to filter the third RF line data frame using a connected components analysis. In some embodiments, the processor circuit is further configured to generate one or more selected biometrics for display on the image output display.

A representative embodiment of a computing system implemented method of detecting blood flow from IVUS and generating a blood flow IVUS image is disclosed, with the computing system including an IVUS console having a processor circuit and an image output display, with the method comprising: using the IVUS console, receiving a plurality of frames of RF line data for the blood flow IVUS image, the plurality of frames of RF line data comprising a first frame of RF line data and a second frame of RF line data; calculating the plurality of correlation RF line data comprising a first frame of turbulence frame and a second frame of turbulence frame; using the processor circuit of the IVUS console, selecting a first subframe of a plurality of first subframes of the first RF line data frame; using a plurality of thresholding and weighting metrics for further focusing on the blood flow motion, using the processor circuit of the IVUS console, selecting a second subframe of a plurality of second subframes of the second RF line data frame for search and comparison, the second subframe larger than the first subframe; using the processor circuit of the IVUS console, performing block matching for each selected first subframe, of the plurality of first subframes, with each corresponding portion of the selected second subframe, of the plurality of second subframes, and generating a similarity score for each selected comparison; using the processor circuit of the IVUS console, and using of a plurality of similarity scores from the block matching, generating a third RF line data frame as a matrix of the plurality of similarity scores; using the processor circuit of the IVUS console, converting the matrix of the plurality of similarity scores to Cartesian coordinates to provide a color image or mask to display as a visual image of the blood flow; using the processor circuit of the IVUS console, generating a brightness-mode (“B-mode”) IVUS image from one or more frames of RF line data of the plurality of frames of RF line data; using a lateral smoothing on color image mask to prevent any artifacts caused from Cartesian coordinate conversion; using the processor circuit of the IVUS console, merging or superimposing the color image or mask on or with the B-mode IVUS image to generate the blood flow IVUS image; and using the processor circuit of the IVUS console, displaying the blood flow IVUS image on the image output display.

In a representative computing system implemented method, the block matching comprises a comparison of at least one first pattern of a plurality of speckle pixels within the selected first subframe with at least one second pattern of a plurality of speckle pixels within the selected second subframe.

In some embodiments, the computing system implemented method further comprises: using the IVUS console, receiving a user selection of or determining one or more signal acquisition modes. In some embodiments, the computing system implemented method further comprises: using the IVUS console, receiving a user selection of blood flow detection and color parameters. In some embodiments, the computing system implemented method further comprises: using the processor circuit of the IVUS console, applying the user-selected blood flow detection and color parameters. In some embodiments, the computing system implemented method further comprises: using the processor circuit of the IVUS console, applying one or more thresholds to RF line data of the first or second frames of RF line data.

In some embodiments, the computing system implemented method further comprises: using the processor circuit of the IVUS console, removing any ringdown artifact from the first and second RF line data frames. In some embodiments, the computing system implemented method further comprises: using the processor circuit of the IVUS console, performing clutter filtering of the first and second RF line data frames. In some embodiments, the computing system implemented method further comprises: using the processor circuit of the IVUS console, providing data padding to the second RF line data frame. In some embodiments, the computing system implemented method further comprises: using the processor circuit of the IVUS console, applying positional (or resolutional) weighting to the first and second RF line data frames.

In some embodiments, the computing system implemented method further comprises: using the processor circuit of the IVUS console, limiting a search region of the second RF line data frame to a region bounded by a vessel contour trace. In some embodiments, the computing system implemented method further comprises: using the processor circuit of the IVUS console, separating the first and second RF line data frames into a plurality of separate search regions.

In some embodiments, the computing system implemented method further comprises: using the processor circuit of the IVUS console, applying spatial and temporal smoothing to the third RF line data frame. In some embodiments, the computing system implemented method further comprises: using the processor circuit of the IVUS console, filtering the third RF line data frame using a connected components analysis. In some embodiments, the computing system implemented method further comprises: using the processor circuit of the IVUS console determining one or more blood flow metrics, the one or more blood flow metrics comprising a displacement determination. In some embodiments, the computing system implemented method further comprises: using the processor circuit of the IVUS console, generating and displaying on the image output display one or more selected biometrics.

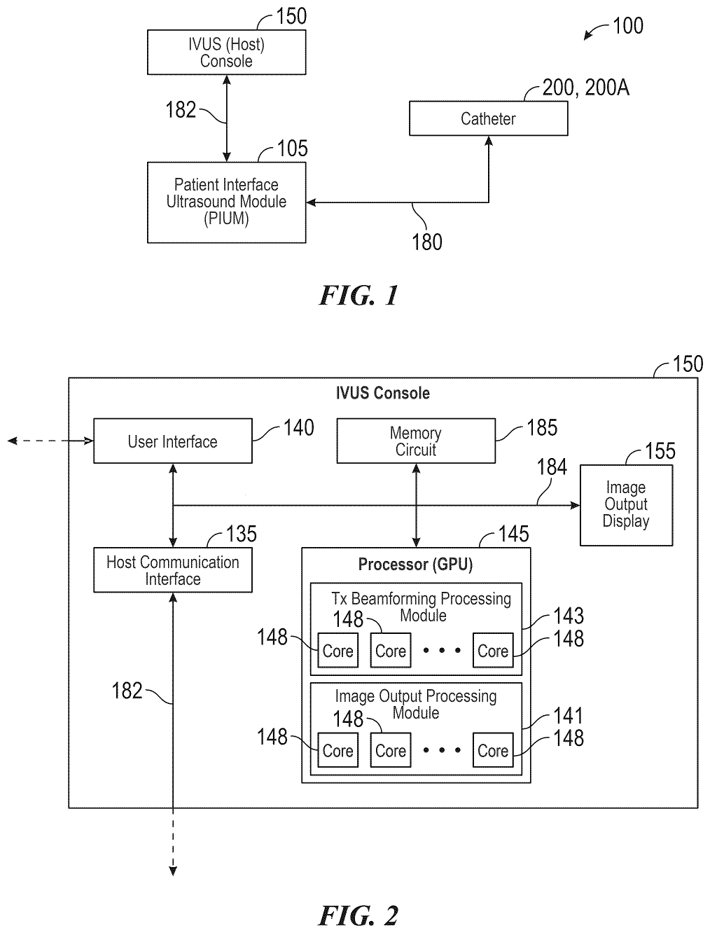

Certain details are set forth in the following description and in FIGS. 1-30 to provide a thorough understanding of various embodiments of the present technology. In other instances, well-known structures, materials, operations, and/or systems often associated with user-interactive vessel contour tracing and/or blood flow measuring in intravascular ultrasound imaging and/or the like are not shown or described in detail in the following disclosure to avoid unnecessarily obscuring the description of the various embodiments of the technology. Those of ordinary skill in the art will recognize, however, that the present technology can be practiced without one or more of the details set forth herein, and/or with other structures, methods, components, and so forth.

The terminology used below is to be interpreted in its broadest reasonable manner, even though it is being used in conjunction with a detailed description of certain examples of embodiments of the technology. Indeed, certain terms may even be emphasized below; however, any terminology intended to be interpreted in any restricted manner will be overtly and specifically defined as such in this Detailed Description section.

The accompanying Figures depict embodiments of the present technology and are not intended to be limiting of its scope unless expressly indicated. The sizes of various depicted elements are not necessarily drawn to scale, and these various elements may be enlarged to improve legibility. Component details may be abstracted in the Figures to exclude details such as the position of components and certain precise connections between such components when such details are unnecessary for a complete understanding of how to make and use the present technology. Many of the details, dimensions, angles, and other features shown in the Figures are merely illustrative of particular embodiments of the disclosure. Accordingly, other embodiments can have other details, dimensions, angles, and features without departing from the present technology. In addition, those of ordinary skill in the art will appreciate that further embodiments of the present technology can be practiced without several of the details described below.

With regard to the terms “distal” and “proximal” within this description, unless otherwise specified, the terms can reference a relative position of the portions of a catheter subsystem with reference to an operator and/or a location in the vasculature. It will be appreciated that such designations refer to the orientation of the referenced component as illustrated in the Figures; the systems of the present technology can be used in any orientation suitable to the user. To the extent any materials incorporated herein by reference conflict with the present disclosure, the present disclosure controls.

### II. Selected Embodiments of IVUS Systems

FIG. 1 is an electrical block diagram illustrating a representative embodiment of an intravascular ultrasound (IVUS) system 100 in accordance with embodiments of the present technology. In the illustrated embodiment, the IVUS system 100 comprises an IVUS catheter 200 which is electrically couplable or connected to a patient interface ultrasound module (“PIUM”) 105 via conductive pins, lines, wires, or a bus 180 (such as via conductive pins 180A of a catheter control connector 210A), which in turn is electrically couplable or connected to an IVUS console 150 (e.g., a host console or a host device) via lines, wires or bus 182. In some embodiments, the IVUS system 100, the IVUS console 150, the PIUM 105, and the IVUS catheter 200 can include several features generally similar or identical in structure and/or function to any of the systems and/or devices described in International Publication No. WO2025/111,572, titled “APPARATUSES, METHODS AND SYSTEMS FOR INTRAVASCULAR ULTRASOUND CIRCUMFERENTIAL SOLID-STATE ARRAY DYNAMIC BEAMFORMING AND METHODS USE,” and filed Nov. 22, 2024, which is incorporated by reference herein in its entirety.

FIG. 2 is an electrical block diagram illustrating a representative embodiment of an IVUS (host) console 150 in accordance with embodiments of the present technology. In the illustrated embodiment, an IVUS console 150 comprises a user interface 140, a host communication interface 135, a processor 145, and an image output display 155. In some embodiments, the IVUS console 150 also typically includes a memory circuit 185, such as to store received data for ultrasound image formation and to store various activation patterns (for loading into the IVUS catheter 200), which are coupled for selected communication via lines, wires or bus 184, for example. The processor 145 generates the data or signaling for the ultrasonic transmit beamforming (using a transmit (Tx) beamforming processing module 143, for example), transmitted through the host communication interface 135 to the PIUM 105 and ultimately to the IVUS catheter 200, 200A. The processor 145 also performs image processing (using an image output processing module 141, for example), generating the data or signaling for the ultrasound image to be displayed on the image output display 155, such as for viewing in real time by medical personnel, using received ultrasonic data transmitted from the IVUS catheter 200 through the PIUM 105 and host communication interface 135. Medical personnel also interact with the IVUS console 150 via the user interface 140, which may be implemented as a keyboard and/or mouse/trackball (not separately illustrated), for example, such as for input and selection of data (such as an energizing sequence) for the transmit beamforming and image selection for output on the image output display 155, also for example.

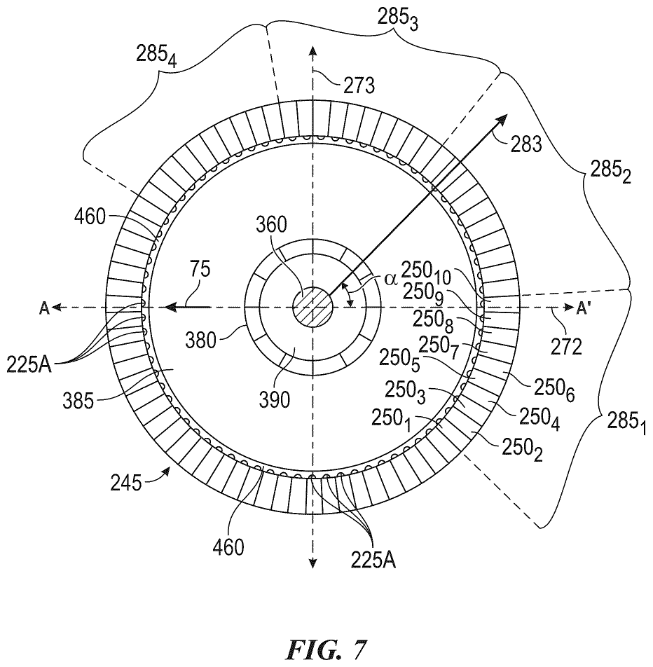

As described in greater detail below, the processor 145 of the IVUS console 150, typically implemented as one or more graphics processing units (“GPUs”) and/or multicore GPUs, performs the vessel contour tracing in the intravascular ultrasound imaging, with the resulting vessel contour trace graphically overlaid or combined with the ultrasound image and displayed on the image output display 155, such as illustrated in FIG. 7. The user typically interacts with the vessel contour trace using the user interface 140 (e.g., a mouse, a touchscreen), such as by adding or moving nodes (or points) of the vessel contour trace. For example, and without limitation, in some embodiments, the method of vessel contour tracing is implemented a plurality of executable instructions (e.g., software) for execution by the processor 145 of the IVUS console 150, together with other operating system software and other display drivers controlling the image output display 155.

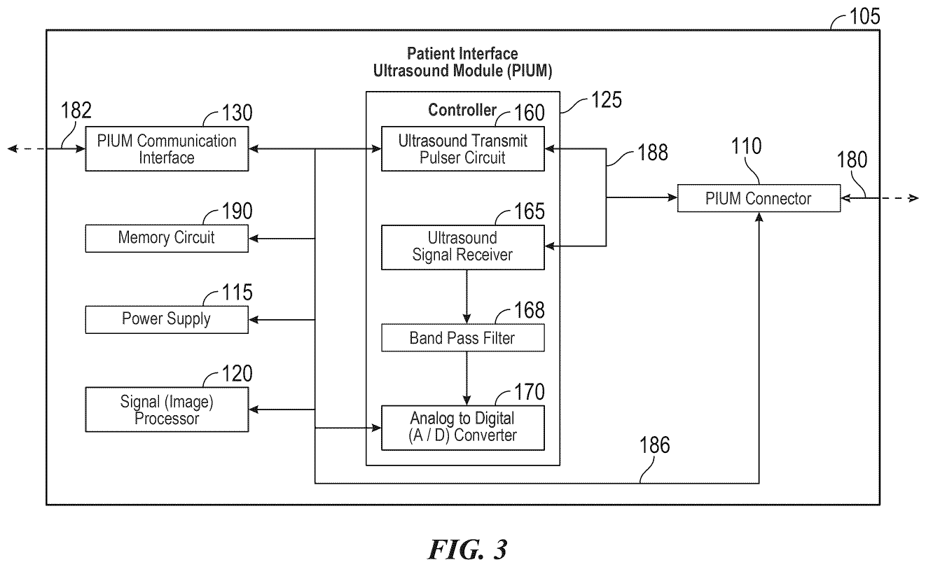

FIG. 3 is an electrical block diagram illustrating a representative embodiment of a PIUM 105 in accordance with embodiments of the present technology. In the illustrated embodiment, the PIUM 105 comprises, for example: a PIUM communication interface 130 (for communication with the IVUS console 150 via the host communication interface 135), a controller 125, a signal (image) processor 120, a power supply 115, and a PIUM connector 110 (for communication (via pins, cable, wires or bus 180, 180A) with the IVUS catheter 200, 200A, typically via a mating or corresponding catheter control connector 210, which are coupled for selected communication via lines, wires or bus 186. In some embodiments, the PIUM 105 also typically includes a memory circuit 190, such as to store received data for ultrasound image formation and possibly also to store various activation patterns (for loading into the IVUS catheter 200), for example. The controller 125 may typically include an ultrasound transmit pulser circuit 160 to generate or provide transmit signals to the IVUS catheter 200, and an “analog front end” comprising an ultrasound signal receiver 165 to receive and amplify signals generated by the IVUS catheter 200, 200A from received ultrasound reflections, a filter such as a bandpass filter 168 to provide bandpass filtering to the received signal, and an analog-to-digital converter (“ADC”) 170, to sample and convert the received analog ultrasound signal to digital ultrasound signal data for signal processing by the signal (image) processor 120 and/or the processor 145, which also may be coupled for selected communication via lines, wires or bus 188, in addition to or in lieu of using lines, wires or bus 186. The bandpass filter 168 may be an analog filter (when arranged to provide bandpass filtering of the received analog signal, as illustrated) or a digital filter (when arranged to provide digital bandpass filtering of the digitized received signal, not separately illustrated). The power supply 115 provides appropriate DC power, ground, and any desired or selected bias voltage to the IVUS catheter 200, 200A, also via the PIUM connector 110 and catheter control connector 210, coupled or couplable via cable, wires, or a bus 180).

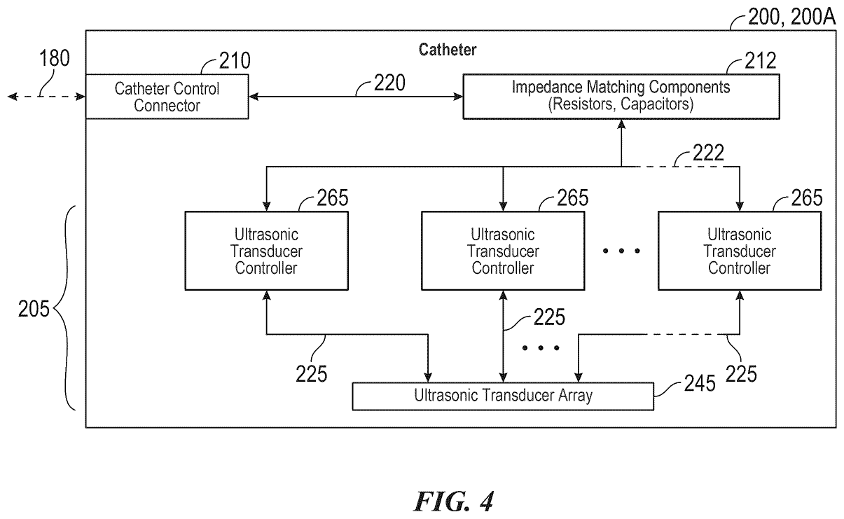

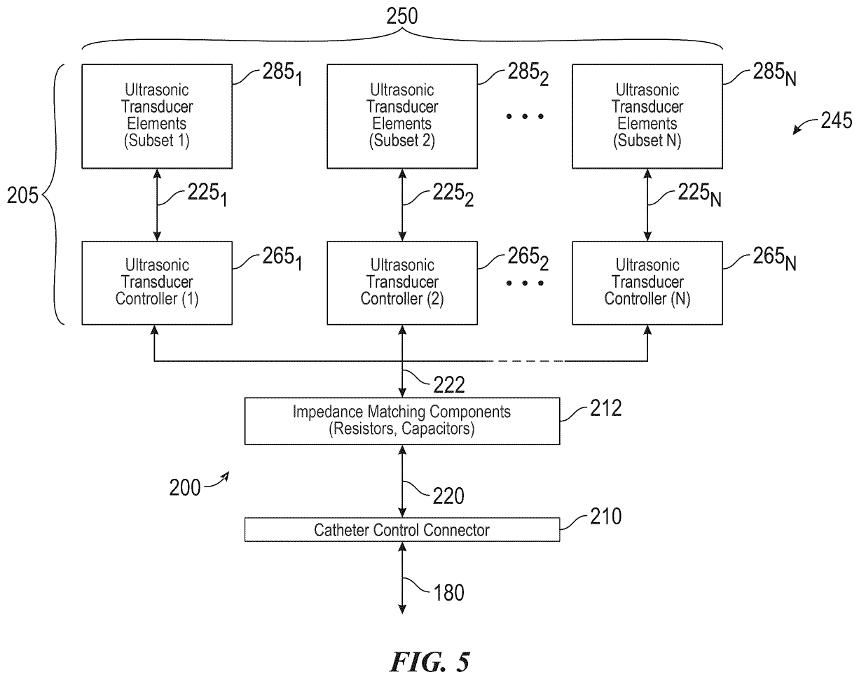

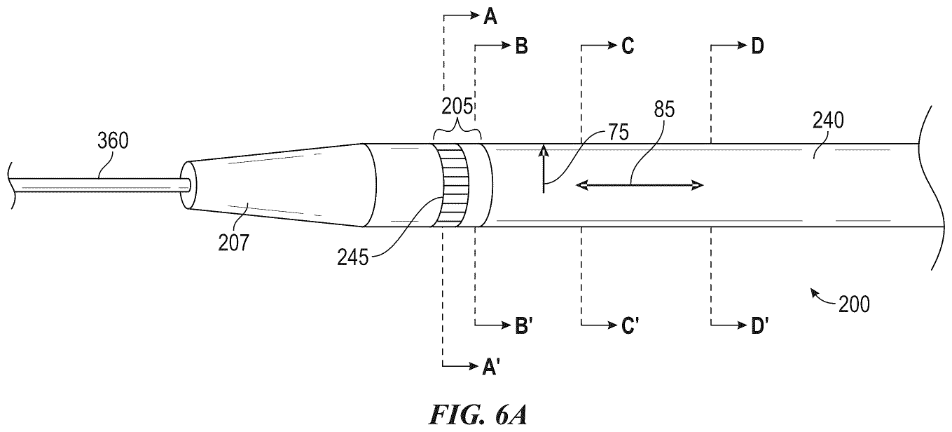

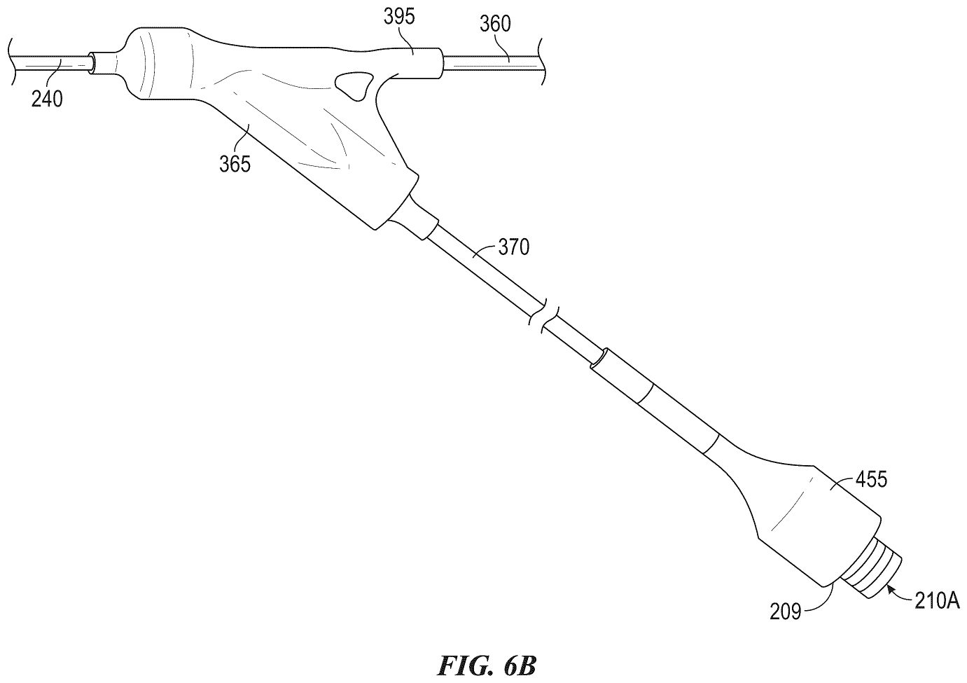

FIG. 4 is a first electrical block diagram illustrating a representative embodiment of an IVUS catheter 200 in accordance with embodiments of the present technology. FIG. 5 is a second, more detailed electrical block diagram illustrating a representative embodiment of an IVUS catheter 200 in accordance with embodiments of the present technology. FIGS. 6A and 6B are isometric views illustrating a representative embodiment of an IVUS catheter 200 in accordance with embodiments of the present technology. FIG. 7 is a cross-sectional view (through the A-A′ plane of FIG. 6A) illustrating a representative circumferential ultrasonic transducer array 245 of the representative embodiment of the IVUS catheter 200 in accordance with embodiments of the present technology.

In the illustrated embodiments, a representative embodiment of an IVUS catheter 200 comprises a catheter housing 240, an ultrasonic transducer assembly 205 (comprising an array 245 of a plurality of ultrasound transducer elements 250 (generally arranged or coupled near a distal end 207 of the IVUS catheter 200) and a plurality of ultrasonic transducer controllers 265 (also arranged within the catheter housing 240), and a catheter control connector 210 (generally arranged or coupled at or near a proximal end 209 of the IVUS catheter 200), for example. The catheter housing 240, in some embodiments, is a flexible, generally elongate (along or defining a longitudinal dimension) and tubular or otherwise cylindrically-shaped housing 240, for example, and may have a form factor and be fabricated as known in the art. The catheter control connector 210 is coupled to or integrally formed with the catheter housing 240, and provides for electrical coupling to the PIUM connector 110 of the PIUM 105 via cable, wires or bus 180, such as via conductive pins 180A. Additional components may also be included in an IVUS catheter 200, such as features or structures for slidably coupling with a catheter guidewire 360, for example.

The IVUS catheter 200 includes a tubular inner wall (or extrusion) 380, with the tubular inner wall 380 being spaced apart radially from the catheter housing 240, toward and generally extending along the central longitudinal axis 85. A first outer lumen 385 is formed between the catheter housing 240 and the tubular inner wall 380. The tubular inner wall (or extrusion) 380 has a second, inner lumen 390. As illustrated in FIGS. 6A and 6B, a guidewire 360 is insertable into the second, inner lumen 390, and the IVUS catheter 200 is thereby slidable along the guidewire 360, such as for following a guidewire inserted into a vein or artery of a subject.

The various signaling, power, and ground wires or cables 220A are arranged within the first, outer lumen 385 of the IVUS catheter 200, for providing signaling, power, and ground to the ultrasonic transducer controllers 265 to selectively energize and receive ultrasound signals from the plurality of the ultrasound transducer elements 250. The various signaling, power, and ground wires or cables 220A terminate in the catheter control connector 210, for coupling to the PIUM 105.

As illustrated in FIG. 6B, the IVUS catheter 200 includes a handle or grip 365 arranged to toward the proximal end 209 of the IVUS catheter 200, for aiding user manipulation of the IVUS catheter 200. As illustrated, the guidewire 360 also passes through a hollow guidewire track 395 of the handle or grip 365, while the various signaling, power, and ground wires or cables 220A are separately arranged within the cable 370 extending from the handle or grip 365 for coupling to the PIUM 105 (via conductive pins 180A).

Referring to FIG. 6A, the plurality of ultrasound transducer elements 250 are arranged adjacent to the ultrasonic transducer controllers 265 and coupled to each other (as described in greater detail below) and to the signaling, power, and ground wires or cables 220A using a flexible circuit board. The plurality of ultrasound transducer elements 250 are coupled to electrically conductive pads 225A, which are electrically coupled (in groups or subsets) to corresponding ultrasonic transducer controllers 265. The signaling, power, and ground wires or cables 220A are coupled to corresponding electrically conductive pads 470, which are electrically coupled (through impedance matching components 212) to the ultrasonic transducer controllers 265.

A representative embodiment of the ultrasonic transducer assembly 205 comprises a plurality of ultrasound transducer elements 250 arranged (typically spaced apart from each other or abutting each other) as a circumferential array 245 and coupled to a plurality of ultrasonic transducer controllers 265, as described in greater detail below. The plurality of ultrasound transducer elements 250 are arranged as a circumferential array 245, with each ultrasound transducer element 250 arranged at a predetermined or fixed radial distance 75 (rather than arranged longitudinally), around or about the catheter housing 240, as illustrated in FIGS. 6A and 6B. Not separately illustrated, the ultrasonic transducer assembly 205 may also include other supporting structure and electrical couplings, as may be needed, such as to space apart the ultrasound transducer elements 250 in the circumferential array 245 and/or further support and secure them to the catheter housing 240, for example. As mentioned above, the ultrasound (or ultrasonic) transducer elements 250 may also be referred to equivalently herein as ultrasound (or ultrasonic) transducers 250. The ultrasound (or ultrasonic) transducer elements 250 may be any type or kind of ultrasound (or ultrasonic) transducer elements, including piezoelectric zirconate transducers (PZTs), capacitive micromachined ultrasonic transducers (CMUTs), and/or piezoelectric micromachined ultrasound transducers, for example, and any and all such variations are considered equivalent and within the scope of the disclosure.

In some embodiments, each ultrasonic transducer controller 265 of the plurality of ultrasonic transducer controllers 265 is implemented as an integrated circuit (“IC”) and is coupled (typically via impedance matching components 212) through corresponding communication lines, wires or bus 220 to the catheter control connector 210, 210A to receive electrical signaling, power, and ground from the PIUM 105, for energizing the ultrasound transducer elements 250 of the ultrasonic transducer assembly 205, for transmitting the ultrasonic signals received from the ultrasound transducer elements 250 of the ultrasonic transducer assembly 205 to the PIUM 105, and for switching or providing power to and signaling from the circumferential array 245 of ultrasound transducer elements 250. The plurality of ultrasonic transducer controllers 265 are arranged and coupled in parallel with each other, between the catheter control connector 210 and the ultrasound transducer elements 250. In various representative embodiments, impedance matching components 212 (such as resistors 214 and capacitors 216) may also be included as an option, with the impedance matching components 212 typically electrically coupled between the ultrasonic transducer controllers 265 and the catheter control connector 210.

Viewing or considering the entire plurality of ultrasound transducer elements 250 to be a “set” of ultrasound transducer elements 250, as described in greater detail below, in some embodiments, the plurality of ultrasound transducer elements 250 are grouped into subsets 285, physically, electrically, and conceptually. In some embodiments, the plurality of ultrasound transducer elements 250 which are arranged in the circumferential array 245 are further grouped physically and electrically into a plurality of subsets 285 of ultrasound transducer elements 250, illustrated in FIGS. 5 and 7 as ultrasound transducer element subset 2851, ultrasound transducer element subset 2852, ultrasound transducer element subset 2853, and so on, through ultrasound transducer element subset 285N. In some embodiments, each ultrasonic transducer controller 265, of the plurality of ultrasonic transducer controllers 265, is coupled through corresponding transmit and receive lines, wires or bus 225 to a selected or corresponding subset 285 of the ultrasound transducer elements 250 of the ultrasonic transducer assembly 205, to selectively and individually address each ultrasound transducer element 250, or selectively address each pair of ultrasound transducer elements 250, of the ultrasound transducer elements 250 of the selected or corresponding subset 285, for example, to generate ultrasound transmission and reception of reflected ultrasound signals for image acquisition.

Each of these ultrasound transducer element subsets 285 is physically (mechanically and electrically) coupled (via corresponding separate busses, wires or lines 2251, 2252, 2253, etc., through 225N) to a separate ultrasonic transducer controller 265, illustrated as ultrasonic transducer controller 2651, ultrasonic transducer controller 2652, and so on, through ultrasonic transducer controller 265N. As such, the control and switching of the ultrasound transducer elements 250 are both electrically and physically (spatially) distributed, using a plurality of individual, separate ultrasonic transducer controllers 265 arranged within the catheter housing 240, 240A, rather than using a single, large integrated circuit having a corresponding large form factor or IC footprint for control and switching. As a result, ultrasonic transducer controller 2651 provides the control and switching of each of the ultrasound transducer elements 250 of ultrasound transducer element subset 2851, ultrasonic transducer controller 2652 provides the control and switching of each of the ultrasound transducer elements 250 of ultrasound transducer element subset 2852, and so on, through ultrasonic transducer controller 265N providing the control and switching of each of the ultrasound transducer elements 250 of ultrasound transducer element subset 285N, such that the plurality of distributed ultrasonic transducer controllers 265 thereby provide the control and switching of all of the ultrasound transducer elements 250 of the entire circumferential array 245 of ultrasound transducer elements 250.

As described in greater detail below, the method of energizing the corresponding ultrasonic transducers 250 and receiving reflected ultrasound signals from the corresponding ultrasonic transducers 250 may be considered to have separate digital and analog phases. The digital phases consist of the selection and switching on or off of the analog switches within the ultrasonic transducer controllers 265. The separate analog phase consists of the transmission of the energizing pulses to the corresponding ultrasonic transducers 250 of the selected sub-aperture 275 and the reception of the reflected ultrasonic signals by the corresponding ultrasonic transducers 250 and transmission of the received ultrasonic signals back to the IVUS console 150 (via the PIUM 105) and/or to the PIUM 105. In the analog phase, while the energizing pulses are being transmitted to the corresponding ultrasonic transducers 250 of the selected sub-aperture 275 and while ultrasound signals are being received by the corresponding ultrasonic transducers 250 of the selected sub-aperture 275, no digital switching is occurring. As a result, the ultrasonic transducer controller(s) 265 is quiescent during the analog phase, and therefore does not generate any digital noise which might potentially interfere with either the transmitted energizing pulses or the received ultrasound signals, providing a significantly greater signal-to-noise ratio than prior art devices.

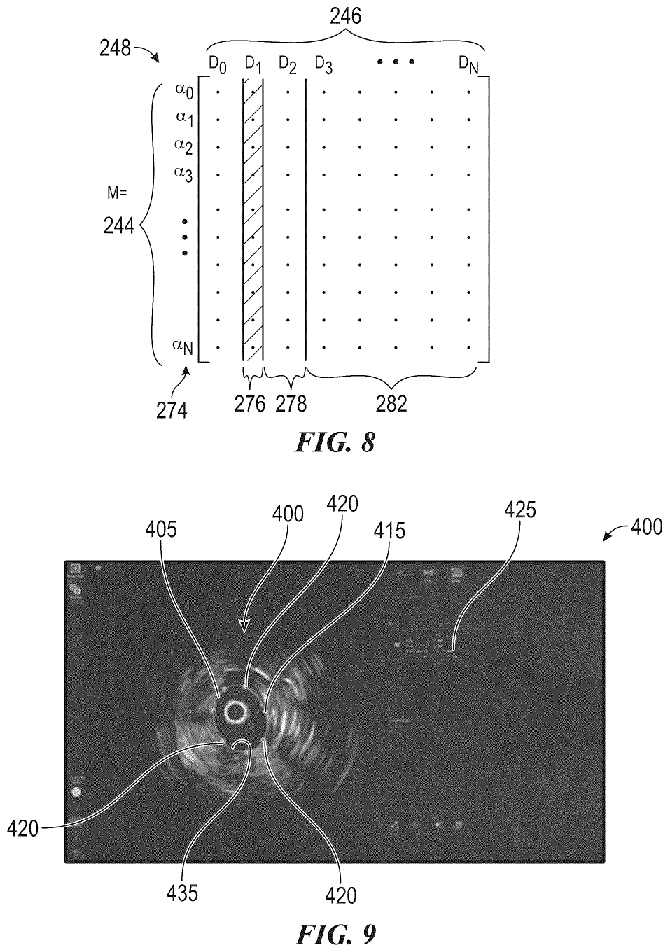

FIG. 8 is a diagram illustrating a frame or matrix of digital ultrasound signal data utilized to form an ultrasound image using the IVUS catheter 200 in accordance with embodiments of the present technology. Referring to FIGS. 7 and 8, in some embodiments, when selected ultrasonic transducers 250 of the selected sub-aperture 275 are energized with the appropriate time delays, the emitted ultrasonic beam is focused along a selected or programmable scan line and has a selectable or programmable focal point, such as a focal point 283 along the scan line 284. FIG. 7 also illustrates overlaid polar coordinates, using radial axis 272 and radial axis 273. Any scan line, such as the scan line 284, may be considered to form an angle “α” (in the radial dimension 75) with respect to the radial axis 272, measured from the longitudinal axis 85 (e.g., the longitudinal center) of the IVUS catheter 200. Following this energizing of the selected ultrasonic transducers 250 to form the scan line 284, reflected ultrasound signals are received and processed. The incoming stream of reflected and received analog ultrasound signals are sampled at predetermined time intervals (e.g., sampled at a particular or selected frequency or sampling rate), with each successive sample converted to digital ultrasound signal data by the PIUM 105, as described above, resulting in a series or sequence of digital ultrasound signal data which is typically referred to as “line data”, “radiofrequency (“RF”) data”, or “RF line data”. This transmission of ultrasound signals, followed by the reception, sampling and conversion of the received analog ultrasound signals to digital ultrasound signal data, is repeated for each selected scan line (e.g., repeated for each angle “α” 274), as selected or programmed by the user. The digital ultrasound signal data (RF line data) provided by the PIUM 105 to the IVUS console 150 for image processing is then characterized in polar coordinates by: (1) the angle “α” 274 (e.g., the angle of the scan line 284 in polar coordinates) for the transmission of the ultrasound signals; and (2) the resulting series or sequence of received, sampled, digital ultrasound signal data obtained following that ultrasound transmission at the selected angle “α” 274.

This series or sequence of received, sampled, digital ultrasound signal data corresponds to the time at which the reflected ultrasound signals are received (and sampled) which, in turn, corresponds to the penetration depth of the reflected ultrasound signal (in the radial dimension, away from the longitudinal axis 85 of the IVUS catheter 200). Stated another way, the transmitted ultrasound signals which are reflected from nearby or adjacent tissue, such as the vessel wall, are received and sampled at or over a first time interval, while the transmitted ultrasound signals which have penetrated further into more distant tissue, such as past the vessel wall and into surrounding tissue, are then reflected and received and sampled at a second, later time interval, while the transmitted ultrasound signals which have penetrated even further into even more distant tissue, such as into subject organs, are then reflected and received and sampled at a third time interval after the second time interval, and so on, for example, forming the series or sequence of received, sampled, digital ultrasound signal data. In addition, it should be noted that other received ultrasound signals, such as the received ultrasound and other signals which result in the ringdown artifact, are typically received and sampled before the transmitted ultrasound signals which are reflected from nearby or adjacent tissue, such as the vessel wall.

As the circumferential array 245 of ultrasound transducer elements 250 is sequentially energized, a plurality of scan lines 284 are formed at different angles “α” 274 and ultrasound signals are emitted around the entire circumferential array 245 (e.g., around the entire three hundred and sixty degrees (360°)), and the corresponding received and sampled digital ultrasound signal data is then used to form a complete image around the IVUS catheter 200, such as illustrated in FIG. 9. Referring to FIG. 8, for any given ultrasound image, the line (or RF) data may then be considered to have the form of a frame or matrix “M” 248, with each row 244 corresponding to the angle “α” 274 (e.g., α₀, α₁, α₂, α₃, . . . α_{N} (with N=359 for spanning the full 360°)), and each column 246 corresponding to the penetration depth of the received ultrasound signal (D₀, D₁, D₂, D₃, . . . D_{N}), as illustrated in FIG. 8. This frame or matrix 248 form of the RF line data is highly useful, as described in greater detail below, for removal of the ringdown artifact and for data padding to reduce or eliminate any stitching artifact.

Referring to FIGS. 1-8, in some embodiments, an IVUS catheter 200 have an array 245 of a plurality of ultrasound (or ultrasonic) transducer elements 250 arranged in a circumferential manner (e.g., spaced-apart circumferentially along or about the IVUS catheter 200) such as illustrated in FIG. 6A (or, stated another way, the ultrasound transducer elements 250 are spaced apart from each other along a circle (or arc) formed at a predetermined or otherwise fixed radial (75) distance from the longitudinal center or axis 85 of the IVUS catheter 200). It should be noted that, in accordance with typical usage in the IVUS imaging field, while each individual ultrasound transducer element 250 is a transducer which emits ultrasound waves and receives reflected ultrasound waves, they are typically referred to as “elements” 250, while the entire totality of ultrasound transducer elements 250 is typically referred to as the overall “transducer”. Accordingly, as utilized herein, an ultrasound transducer element 250 or element 250 refers to the single, individual ultrasound transducer element (e.g., a piezoelectric zirconate transducer (PZT) element), while the collective group of elements 250 is referred to herein as an array 245 of ultrasound transducer elements 250 (e.g., an ultrasound transducer array 245). Also in accordance with typical usage in the optical and imaging fields, for a compound device such as an IVUS catheter 200 having a plurality of individually selectable ultrasound transducer elements 250, as described in greater detail below, selected subsets of the plurality of the ultrasound transducer elements 250 are referred to as “sub-apertures” 275; however, these selected subsets of the plurality of the ultrasound transducer elements 250 may be referred to equivalently as “apertures”, and any and all such variations are considered equivalent and within the scope of the disclosure.

Specifically, in the various representative embodiments, not all of the ultrasound transducer elements 250 of the IVUS catheter 200 are energized and transmitting at the same time. Instead, a subset of the plurality of the ultrasound transducer elements 250 are energized, in groups referred to as sub-apertures (or, equivalently, apertures) 275, either with each ultrasound transducer element 250 of any selected sub-aperture 275 individually and separately energized in a selected sequence with a selected delay (e.g., the energizing of the element 250 having an offset time from the energizing of other ultrasound transducer elements 250 of the selected sub-aperture 275), or with symmetrical pairs of ultrasound transducer elements 250 of any selected sub-aperture 275 collectively and simultaneously energized in a selected sequence with a selected delay (e.g., the energizing of the pair of elements 250 having an offset time from the energizing of other ultrasound transducer elements 250 of the selected sub-aperture 275). This energizing sequence of the ultrasound transducer elements 250 of the selected sub-aperture 275 with selected delays creates geometric delays in the energizing of the ultrasound transducer elements 250 and resulting ultrasound transmission, thereby generating a corresponding ultrasonic wavefront pattern resulting in a dynamically focused ultrasonic beam.

As used herein, a processor 145, a signal processor 120, and a controller 125 may be implemented using any type of digital or analog electronic or other circuitry which is arranged, configured, designed, programmed or otherwise adapted to perform any portion of the signal processing, image generation, and beamforming (including delay calculations) described herein. As the term processor and/or controller is used herein, a processor 145, a signal processor 120, and/or a controller 125 may include use of a single integrated circuit (“IC”), or may include use of a plurality of integrated circuits or other electronic components connected, arranged or grouped together, such as processors, controllers, microprocessors, digital signal processors (“DSPs”), parallel processors, multiple core processors, custom ICs, application specific integrated circuits (“ASICs”), field programmable gate arrays (“FPGAs”), adaptive computing ICs, discrete electronic components, and any associated memory (such as RAM, DRAM and ROM), and other ICs and components, whether analog or digital. As a consequence, as used herein, the term processor should be understood to equivalently mean and include a single IC, or arrangement of custom ICs, ASICs, processors, microprocessors, controllers, FPGAs, adaptive computing ICs, or some other grouping of integrated circuits or discrete electronic components which perform the functions described above and further described below, and may further include any associated memory, such as microprocessor memory or additional RAM, DRAM, SDRAM, SRAM, MRAM, ROM, FLASH, EPROM or E²PROM. A processor 145, a signal processor 120, and/or a controller 125, with any associated memory, may be arranged, adapted or configured (via programming, FPGA interconnection, or hard-wiring) to perform any portion of the signal processing, image generation, and beamforming of the present disclosure, as described herein. For example, the methodology may be programmed and stored, in a processor 145, a signal processor 120, and/or a controller 125 with its associated memory (and/or memory 185, 190, respectively) and other equivalent components, as a set of program instructions or other code (or equivalent configuration or other program) for subsequent execution when the processor 145, signal processor 120, and/or controller 125 is operative (e.g., powered on and functioning). Equivalently, when the processor 145, signal processor 120, and/or controller 125 may implemented in whole or part as FPGAs, custom ICs and/or ASICs, the FPGAs, custom ICs or ASICs also may be designed, configured and/or hard-wired to implement any portion of the personalization of search results and search result rankings of the present disclosure. For example, the processor 145, signal processor 120, and/or controller 125 may be implemented as an arrangement of analog and/or digital circuits, controllers, microprocessors, DSPs and/or ASICs, collectively referred to as a “processor”, or “controller” which are respectively hard-wired, arranged, programmed, designed, adapted or configured to implement signal processing, image generation, and beamforming of the present disclosure, including possibly in conjunction with a memory 185, 190.

A memory 185, 190 and/or activation pattern registers 305 may be embodied as any type of data storage device, such as RAM, FLASH, DRAM, SDRAM, SRAM, MRAM, FeRAM, ROM, EPROM or E²PROM, and is utilized for data storage, and also may be utilized to store any data, activation patterns, program instructions or configurations which may be utilized by a processor 145, a signal processor 120, a controller 125, and/or activation pattern selection logic 310. More specifically, the memory 185, 190 and/or activation pattern registers 305 may be embodied in any number of forms, including within any nontransitory, machine-readable data storage medium, memory device or other storage or communication device for storage or communication of information, currently known or which becomes available in the future, including, but not limited to, a memory integrated circuit (“IC”), or memory portion of an integrated circuit (such as the resident memory within a processor 145, signal processor 120, and/or controller 125), whether volatile or non-volatile, whether removable or non-removable, including without limitation RAM, FLASH, DRAM, SDRAM, SRAM, MRAM, FeRAM, ROM, EPROM or E²PROM, or any other form of memory or data storage device, as the case may be (depending upon various form factors, for example), such as a magnetic hard drive, an optical drive, a magnetic disk or tape drive, a hard disk drive, other machine-readable storage or memory media such as a floppy disk, a CDROM, a CD-RW, digital versatile disk (DVD) or other optical memory, or any other type of memory, storage medium, or data storage apparatus or circuit, which is known or which becomes known, depending upon the selected embodiment. The memory 185, 190 and/or activation pattern registers 305 may store data in any way or configuration, including as various look up tables, parameters, coefficients, databases, other information and data, programs or instructions (of the software of the present technology), and other types of tables such as database tables or any other form of data repository.

The communication and user interface (I/O) circuits 130, 135, 140, 300 may be implemented as known or may become known in the art, and may include impedance matching capability, voltage rectification circuitry, voltage translation for a low voltage processor to interface with a higher voltage control bus for example, various switching mechanisms (e.g., transistors) to turn various lines or connectors on or off in response to signaling from a processor 145, signal processor 120, and/or controller 125, other control logic circuitry, and/or physical coupling mechanisms. In addition, the communication and user interface (I/O) circuits 130, 135, 140, 300 are also configured to receive and/or transmit signals, such as through hard-wiring or RF signaling, for example, to receive and transmit information in real-time, also for example. The communication and user interface (I/O) circuits 130, 135, 140, 300 are utilized for appropriate connection to a relevant channel, network or bus; for example, the communication and user interface (I/O) circuits 130, 135, 140, 300 may provide impedance matching, drivers and other functions for a wireline interface, may provide demodulation and analog to digital conversion for a wireless interface, and may provide a physical interface for the memory 185, 190 and/or activation pattern registers 305 with other devices. In general, the communication and user interface (I/O) circuits 130, 135, 140, 300 are used to receive and transmit data, depending upon the selected embodiment, including activation patterns, control messages, and other pertinent information.

As indicated above, the processor 145, signal processor 120, controller 125, and/or activation pattern selection logic 310 is or are hard-wired, configured or programmed, using software and data structures of the technology, for example, to perform any portion of the signal processing, image generation, and beamforming, of the present disclosure. As a consequence, portions of the system and method of the present disclosure may be embodied as software which provides such programming or other instructions, such as a set of instructions and/or metadata embodied within a nontransitory computer-readable medium, described above. In addition, metadata may also be utilized to define the various data structures of a look up table or a database. Such software may be in the form of source or object code, by way of example and without limitation. Source code further may be compiled into some form of instructions or object code (including assembly language instructions or configuration information). The software, source code or metadata of the present technology may be embodied as any type of code, such as C, C++, C#, Javascript, Adobe Flash, Silverlight, SystemC, LISA, XML, Java, Brew, SQL and its variations (e.g., SQL 99 or proprietary versions of SQL), DB2, Oracle, or any other type of programming language which performs the functionality described herein, including various hardware definition or hardware modeling languages (e.g., Verilog, VHDL, RTL) and resulting database files (e.g., GDSII). As a consequence, “software”, “program”, “computer program”, or a “module”, “program module”, “software module”, as used equivalently herein, means and refers to any programming language, of any kind, with any syntax or signatures, which provides or can be interpreted to provide the associated functionality or methodology specified (when instantiated or loaded into a processor or computer and executed, including the processor 145, signal processor 120, controller 125, and/or activation pattern selection logic 310, for example). In addition, any of such program or software modules may be combined or divided in any way. For example, a larger module combining first and second functions is considered equivalent to a first module which performs the first function and a separate second module which performs the second function.

In some embodiments, the computing devices and systems on which the system can be implemented can include a central processing unit, input devices, output devices (e.g., display devices and speakers), storage devices (e.g., memory and disk drives), network interfaces, graphics processing units, accelerometers, cellular radio link interfaces, global positioning system devices, and/or the like. The input devices can include keyboards, pointing devices, touchscreens, gesture recognition devices (e.g., for air gestures), thermostats, smart devices, head and eye tracking devices, microphones for voice or speech recognition, and/or the like. The computing devices and systems can include desktop computers, laptops, tablets, e-readers, personal digital assistants, smartphones, gaming devices, servers, and computer systems such as massively parallel systems. The computing devices and systems can each act as a server or client to other server or client devices. The computing devices and systems can access computer-readable media that include computer-readable storage media and data transmission media. The computer-readable storage media are tangible storage means that do not include transitory, propagating signals. Examples of computer-readable storage media include memory such as primary memory, cache memory, and secondary memory (e.g., CD, DVD, Blu-Ray), and include other storage means. Moreover, data may be stored in any of a number of data structures and data stores, such as databases, files, lists, emails, distributed data stores, storage clouds, and/or the like.

The computer-readable storage media can be recorded upon or encoded with computer-executable instructions or logic that implements the system, such as a component comprising computer-executable instructions stored in one or more memories for execution by one or more processors. In addition, the stored information can be encrypted. The data transmission media are used for transmitting data via transitory, propagating signals or carrier waves (e.g., electromagnetism) via a wired or wireless connection. In addition, the transmitted information can be encrypted. Various communications links can be used, such as the Internet, a local area network, a wide area network, a point-to-point dial-up connection, a cell phone network, and/or the like for connecting the computing systems and devices to other computing systems and devices to send and/or receive data, such as via the Internet or another network and its networking hardware, such as switches, routers, repeaters, electrical cables and optical fibers, light emitters and receivers, radio transmitters and receivers, and/or the like. While computing systems and devices configured as described above are typically used to support the operation of the system, those skilled in the art will appreciate that the system can be implemented using devices of various types and configurations, and having various components. Accordingly, computing steps, computing methods, computing operations, and/or the like described herein can be implemented locally at the IVUS system 100 (e.g., at the IVUS console 150) and/or remotely (e.g., via a cloud or other remote computing system).

The systems can be described in the general context of computer-executable instructions, such as program modules and components, executed by one or more computers, processors, or other devices, including single-board computers and on-demand cloud computing platforms. Generally, program modules or components include routines, programs, objects, data structures, and/or the like that perform particular tasks or implement particular data types. Typically, the functionality of the program modules can be combined or distributed as desired in various embodiments. Aspects of the system can be implemented in hardware using, for example, an application-specific integrated circuit (“ASIC”).

### III. Selected Embodiments of Vessel Contour Tracing Systems

FIG. 9 is a photograph illustrating a first ultrasound image 400 of a vein 415 and illustrating an overlaid, representative first user-interactive vessel contour trace 405 in accordance with embodiments of the present technology.

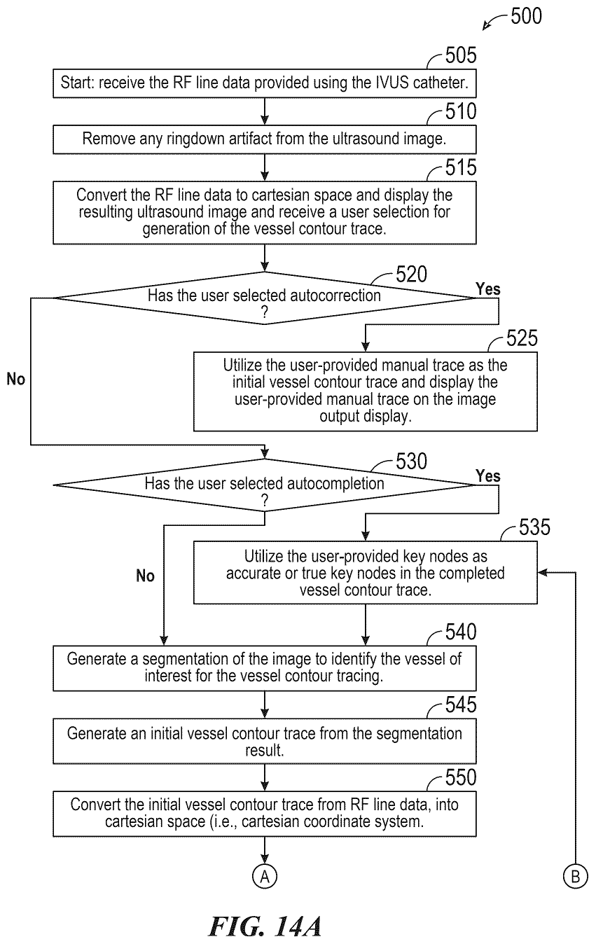

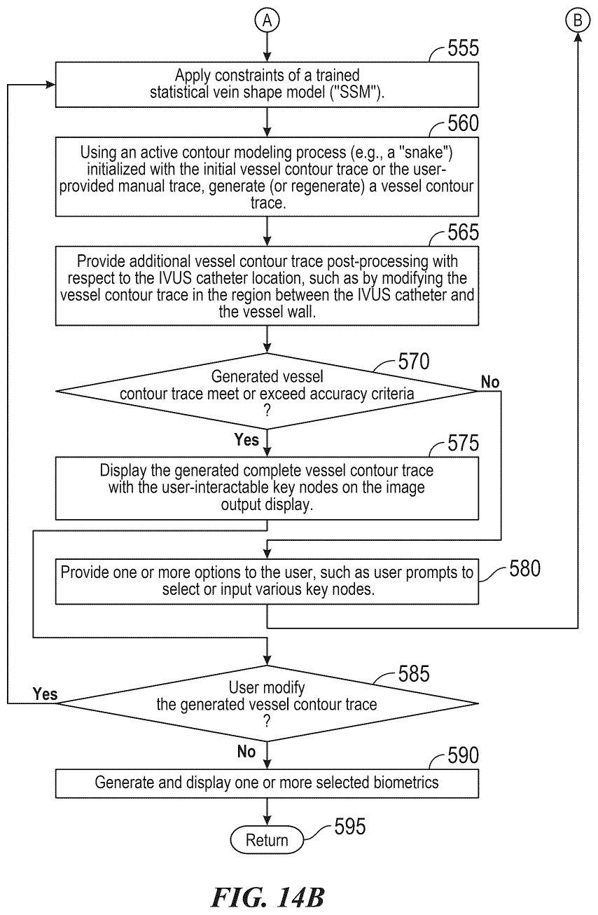

Referring to FIG. 9, a first method of user interaction with the vessel contour trace is illustrated. For this embodiment, the user can select or click on a graphical user interface (e.g., a GUI button) (not separately illustrated), and the processor 145 will generate (and display on the image output display 155) a complete vessel contour trace 405 (provided that the complete vessel contour trace 405 meets various accuracy criteria, such as quality metrics, described below). The generation of the complete vessel contour trace 405 by the processor 145 (with corresponding display of the complete vessel contour trace 405 on the image output display 155) is described in greater detail with reference to FIGS. 14A-20B. In some embodiments, the user-interactive vessel contour trace 405 may also include a plurality of control points, referred to herein as key nodes 420, with which the user also may interact to modify or otherwise edit the complete vessel contour trace 405, such as by selecting (e.g., clicking on) a selected key node 420 and dragging and dropping the selected key node 420 to a new location or by adding additional key nodes 420 (such as by clicking on a user-selected point on the IVUS image) (both of which, as described in greater detail below, will modify the displayed vessel contour trace 405). The image output display 155 of the IVUS console 150 displays the ultrasound image 400 with the complete vessel contour trace 405, and may also display or include other information, such as the various biometrics 425 described above. It should be noted that any vessel contour trace provided by the various apparatus, system, and method embodiments, as described in greater detail below, will have met or exceeded various quality metrics or other suitable accuracy criteria. Once the user considers the vessel contour trace 405 to be acceptable, the user can select or click on a graphical user interface (e.g., a GUI button) (not separately illustrated) to confirm the selected vessel contour trace and the selected vessel contour trace will be available for further operations, such as biometric measurement calculations.

In various representative embodiments, the user may interact with the displayed vessel contour trace 405 to determine the various biometrics. For example, the user may select and click on various points of the displayed vessel contour trace 405 for desired measurements, such as clicking on two points of the displayed vessel contour trace 405, and the processor 145 providing a ruler or a distance measurement between those two selected points. A quality indicator may also be displayed with the vessel contour trace 405, such as a numerical score or displaying the vessel contour trace 405 in a particular color, such as to indicate goodness of fit.

As the displayed vessel contour traces are generated for a plurality of IVUS images along a vessel (during IVUS catheter 200 pullback), the processor 145 of the IVUS console 150 may also use the plurality of vessel contour traces to generate three-dimensional (3D) images of the vessel, for example. Temporal or spatial smoothing may also be applied (to the two-dimensional or to the 3D images) by the processor 145 of the IVUS console 150, also for example. In addition, other views, such as sagittal, coronal, and cross-section views, also may be generated from the plurality of vessel contour traces by the processor 145 of the IVUS console 150.

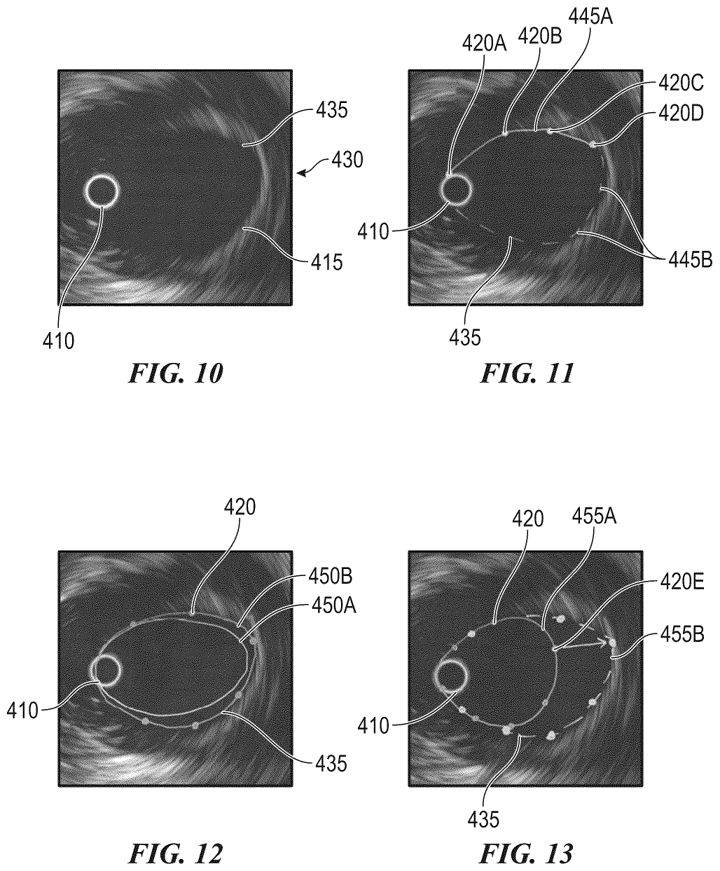

FIG. 10 is a photograph illustrating a second ultrasound image 430 of a vein 415 and illustrating a ringdown artifact 410. FIG. 11 is a photograph illustrating the second ultrasound image 430 of a vein 415 of FIG. 10 and illustrating an overlaid, representative second user-interactive vessel contour trace 445 (divided into vessel contour traces 445A, 445B) in accordance with embodiments of the present technology. FIG. 12 is a photograph illustrating the second ultrasound image 430 of a vein 45 of FIG. 10 and illustrating an overlaid manual vessel contour trace 450A together with an overlaid, representative third user-interactive vessel contour trace 450B in accordance with embodiments of the present technology. FIG. 13 is a photograph illustrating the second ultrasound image 430 of a vein 415 of FIG. 10 and illustrating an overlaid fourth user-interactive vessel contour trace 455A together with an overlaid, representative fifth user-interactive vessel contour trace 455B in accordance with embodiments of the present technology. Referring to FIGS. 10-13, the illustrated embodiments serve to illustrate various additional ways in which the user may interact with and manipulate the various vessel contour traces in accordance with the representative embodiments, and further illustrate the correct or proper formation of a vessel contour trace between the vessel wall 435 and the IVUS catheter 200 and through the ringdown artifact 410.

Referring to FIG. 11, a second method of user interaction with the vessel contour trace, referred to as “autocompletion,” is illustrated. One of the unique ways of representing the vessel contour trace is to use interactable key nodes 420, as mentioned above. Instead of drawing a complete contour, the user can initiate the vessel contour tracing function by clicking on points or nodes (420A, 420B, 420C, 420D) as identified by the user adjacent the vessel wall, and the processor 145 will complete the vessel contour trace 445A and continue with generating the complete vessel contour trace (445A together with 445B). Alternatively and equivalently, the user can initiate the vessel contour tracing function by clicking on points or nodes (420A, 420B, 420C, 420D) as identified by the user on the vessel contour trace 445A (which may be generated by either the processor 145 or by the user. The autocompletion method performed by the processor 145 takes those user-added points as true or accurate key nodes 420 and then generates and displays a suggested complete vessel contour trace (445A together with 445B) with those key nodes 420A, 420B, 420C, 420D (and potentially more key nodes if the vessel contour trace is not fully described by the user-provided key nodes). The generation of the complete vessel contour trace 445A, 445B by the processor 145 (with corresponding display of the complete vessel contour trace 445A, 445B on the image output display 155) is described in greater detail with reference to FIGS. 14A-20B. If the user is not satisfied with the current vessel contour trace suggestion, any further modifications such as adding new a key node 420 or dragging a current key node 420 to another position are accepted and will trigger the performance of autocompletion by the processor 145 again, with the processor 145 providing (and displaying on the image output display 155) a new suggested vessel contour trace based on all the key nodes 420 added or modified by the user. Once the user considers the vessel contour trace (445A together with 445B) to be acceptable, the user can select or click on a graphical user interface (e.g., a GUI button) (not separately illustrated) to confirm the selected vessel contour trace and the selected vessel contour trace will be available for further operations, such as biometric measurement calculations.

Referring to FIG. 12, a third method of user interaction with the vessel contour trace, referred to as “autocorrection”, is illustrated. In this third method, the user manually or roughly provides an initial vessel contour trace 450A, which may or may not be very accurate (e.g., by drawing a vessel contour trace on a touch screen with a finger or a stylus). In this case, the autocorrection methodology provides a suggested or corrected complete vessel contour trace 450B. The generation of the complete vessel contour trace 450B by the processor 145 (with corresponding display of the complete vessel contour trace 450B on the image output display 155) is described in greater detail with reference to FIGS. 14A-20B. Once the corrected vessel contour trace 450B is displayed, the user can choose to accept it or reject it. The original user-drawn vessel contour trace 450A may continue to be displayed as well, and the user may choose to accept the original vessel contour trace 450A, or the corrected vessel contour trace 450B, or reject both and reset the process by drawing a new vessel contour trace, for example, followed by the processor 145 generating another, corrected complete vessel contour trace 450B. Once the user considers the vessel contour trace 450B to be acceptable, the user can select or click on a graphical user interface (e.g., a GUI button) (not separately illustrated) to confirm the selected vessel contour trace 450B and the selected vessel contour trace will be available for further operations, such as biometric measurement calculations.

Referring to FIG. 13, a fourth method of user interaction with the vessel contour trace, referred to as “simultaneous suggestion”, is illustrated, and is a variant of the method described above. In this fourth method, the generation of the vessel contour trace 455A commences without any initialization inputs from the user (e.g., without the user clicking on the image to create any key nodes 420). For this embodiment, the user can select or click on a graphical user interface (e.g., a GUI button) (not separately illustrated), and the processor 145 will generate (and display on the image output display 155) a complete vessel contour trace 455A, with interactable key nodes 420. The generation of the complete vessel contour trace 450B by the processor 145 (with corresponding display of the complete vessel contour trace 450B on the image output display 155) is described in greater detail with reference to FIGS. 14A-20B. Similarly to the auto-completion method, tracing is triggered each time the user adds a new key node 420 or drags and drops an existing key node 420 to another location, such as dragging and dropping the key node 420E as illustrated, forming the revised or corrected vessel contour trace 455B. Unless the user chooses to further modify a key node 420, any key node 420 already modified by the user is considered to be accurate and will not be moved or modified in the newly revised or corrected vessel contour trace 455B. Once the user considers the vessel contour trace 455B to be acceptable, the user can select or click on a graphical user interface (e.g., a GUI button) (not separately illustrated) to confirm the selected vessel contour trace 455B and the selected vessel contour trace will be available for further operations, such as biometric measurement calculations.

In operation, the user selects the IVUS image for complete vessel contour tracing. In general, the user should select an IVUS image which is comparatively clear and shows a vessel wall, with or without blood clots or other occlusions. Once the user has selected an IVUS image and selected vessel contour tracing, the processor 145 will generate the complete vessel contour trace, with corresponding display of the complete vessel contour trace on the image output display 155, without further user involvement. If no editing activities are performed by the user, the complete vessel contour trace is utilized for further processes, such as biometric measurement calculations. In addition, the user may also freely edit the complete vessel contour trace, as indicated above, such as by interacting with (dragging, dropping, adding) the various control points referred to as key nodes 420.

In some embodiments, once the user has accepted the vessel contour trace, the processor 145 will determine and/or update various biometric measurement calculations including, for example, vein/vessel area, minimum diameter, maximum diameter, average diameter effective diameter, global maximum area, global minimum area, fractional occlusion (clot) burden, percent occlusion. For example, as a user may edit the complete vessel contour trace, the processor 145 may update any or all of these biometric measurement determinations. In addition, the processor 145 of the IVUS console 150 may also identify or flag which IVUS images indicate the presence of occlusions, for example.

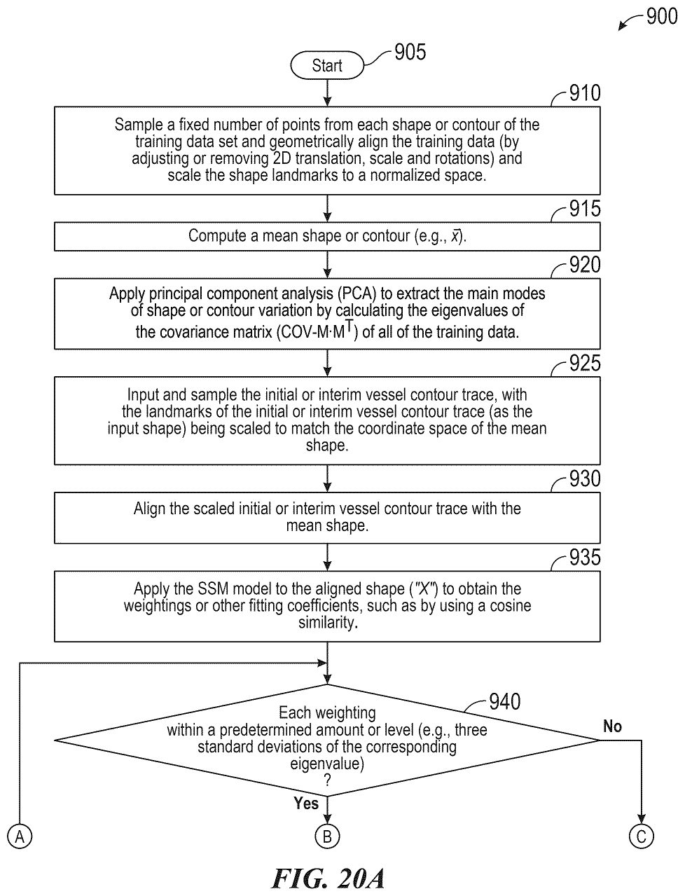

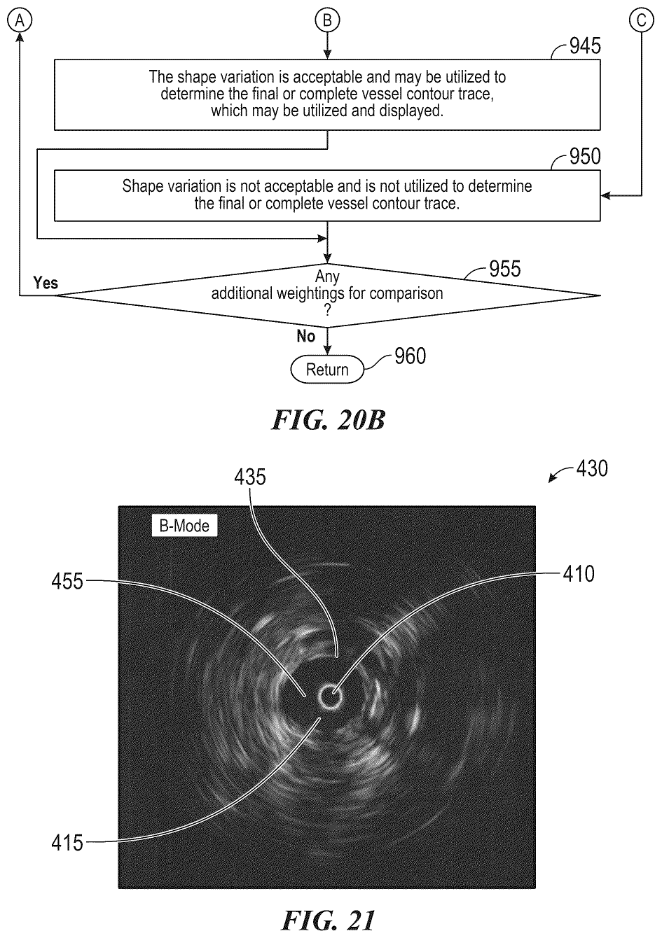

As mentioned above, vessel contour traces are provided by the various embodiments provided that the suggested vessel contour trace is sufficiently accurate (e.g., meets or exceeds selected quality metrics), as described in greater detail below with reference to FIGS. 20A and 20B. If the various embodiments are unable to provide an accurate vessel contour trace, such as due to the quality of the IVUS image, the various embodiments may also prompt the user to initiate the vessel contour trace, such as by clicking on regions of the image to form key nodes 420 or manually drawing an initial vessel contour trace. Manual tracing can also override the above process at any time, and it would reset any previous input.

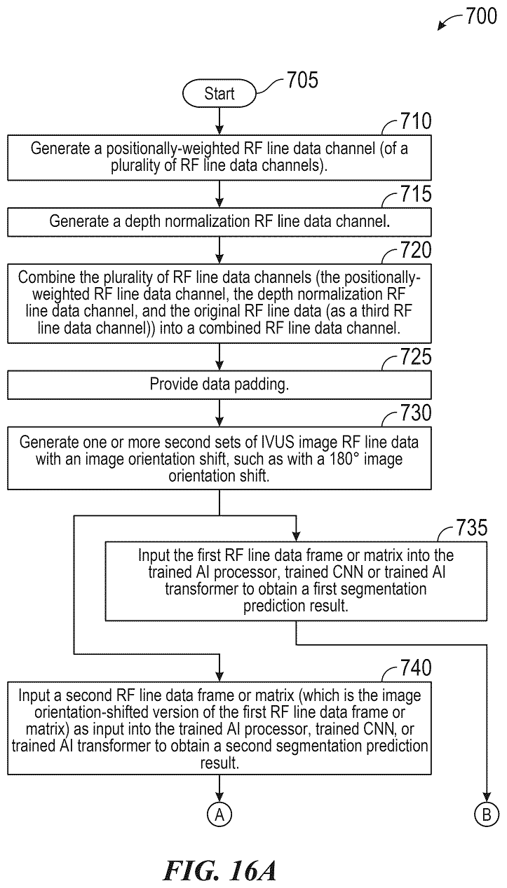

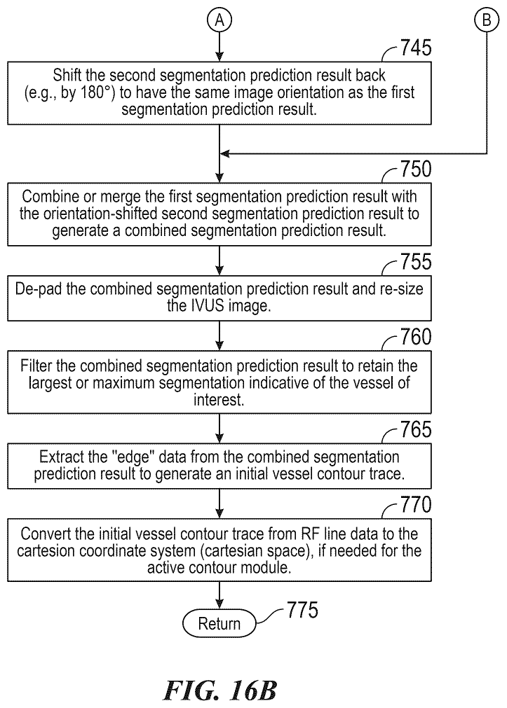

The complete vessel contour tracing method is explained in FIGS. 14A and 14B, and essentially consists of three modules. The first, a deep learning-based segmentation module, will have been pretrained using a supervised method on manual labels, which roughly segments the vein area from the rest of an IVUS image and generates an initial vessel contour trace. It should be noted that the processor 145 is configured as a trained processor, such as a trained artificial intelligence (“AI”) processor, a trained neural network (e.g., a trained convolutional neural network (“CNN”)), or as a trained AI vision-based transformer, for example (with additional available implementations discussed in greater detail below). The accurate image segmentation data has been generated by having trained professionals: (1) review a plurality of ultrasound images (displayed with Cartesian coordinates (e.g., in Cartesian space)) provided using the IVUS catheter; and (2) identify and mark features within each such ultrasound image (such as veins (with and without blood clots), arteries (with and without blood clots), collateral veins, stents, and other features). It should be noted that these IVUS images utilized in training intentionally include ultrasound artifacts, such as acoustic shadowing, blackout regions to account for dead elements, or comet-tail artifacts around the vessel walls, for example. This plurality of ultrasound images with the segmentations may also be re-converted back to RF line data, for use in training (or, alternatively, may remain in Cartesian space). In some embodiments, the plurality of ultrasound images with their professional, marked (e.g., manually labelled) segmentations are converted back into corresponding RF line data (with the segmentations), and with the additional processing steps 710 through 750 illustrated in FIGS. 16A and 16B, are then utilized as corresponding segmentation “masks” in the segmentation process. This plurality of ultrasound images with their professional, marked (e.g., manually labelled) segmentations may also be utilized to form the various vessel contour traces utilized for the training data set in the SSM.

For segmentation training, each image of the plurality of ultrasound images (either as RF line data or as an image in Cartesian space) is processed as described in greater detail below (processing steps 710 through 750 illustrated in FIGS. 16A and 16B) and is then passed through the AI processor, CNN or transformer to result in a predicted image segmentation, which is compared with the accurate image segmentation data (the RF line data or Cartesian space data), compared with the professional, marked (e.g., manually labelled) segmentations, and any resulting error is utilized to adjust corresponding weightings or other parameters utilized in the AI processor, CNN or transformer. For example, the weights to improve prediction or inferential accuracy during the next iteration may be adjusted using a stochastic backpropagation method which calculates a gradient of a loss function with respect to current weights. This process is repeated many times with all the images inside the training dataset until the model becomes good at predicting the correct segmentation, e.g., this training process continues until the selected or desired level of accuracy is achieved. As indicated above, this accurate segmentation data (the professional, marked (e.g., manually labelled) segmentations) is then converted from the Cartesian space into a corresponding plurality of “masks” comprised of RF line data, and utilized in the segmentation process on new IVUS images to generate an initial vessel contour trace.

The second module uses an active contour modelling process (e.g., a “snake”) to refine the initial vessel contour trace by finding the precise vein edges using gradient, brightness changes, as well as shape constrains to iteratively improve the vessel contour trace from the initial vessel contour trace provided by the segmentation module, generating an interim vessel contour trace. The third module uses a Statistical vein Shape Model (SSM) to regularize or constrain the interim vessel contour trace provided by the active contour modelling process, and make sure it is consistent with the statistical distribution of true vein shapes. Combining the active contour modelling process (e.g., a “snake”) with this additional statistical shape modelling provides a high degree of regularity and reproducibility of the resulting complete vessel contour trace, particularly compared with end-to-end AI-based methods. The SSM model has also been trained on prelabeled vein shapes. The SSM model also checks the final complete vessel contour trace and determines whether it is sufficiently accurate (meeting quality criteria) to be useable and displayed to the user, as mentioned above. Each module is described in greater detail below.

FIGS. 14A and 14B are flow diagrams illustrating a computing system implemented method 500 to generate and display a user-interactive vessel contour trace in an ultrasound image provided using the IVUS catheter 200 in accordance with embodiments of the present technology. As mentioned above, the IVUS console 150 (having and using the processor 145, the memory circuit 185, and the image output display 155) provides the computing system which implements the method 500 to generate and display a user-interactive vessel contour trace in an ultrasound image provided using the IVUS catheter 200. As indicated above, the processor 145 is configured as a trained AI processor, or a trained neural network (e.g., a trained convolutional neural network (“CNN”)) or as a trained artificial intelligence (“AI”) vision-based transformer, any or all of which have been trained using accurate image segmentation data, and that the memory circuit 185 is configured to store either or both such accurate image segmentation data utilized in the training and/or the weightings provided to the AI processor, CNN or transformer from the training, described in greater detail below with reference to FIGS. 16A and 16B.

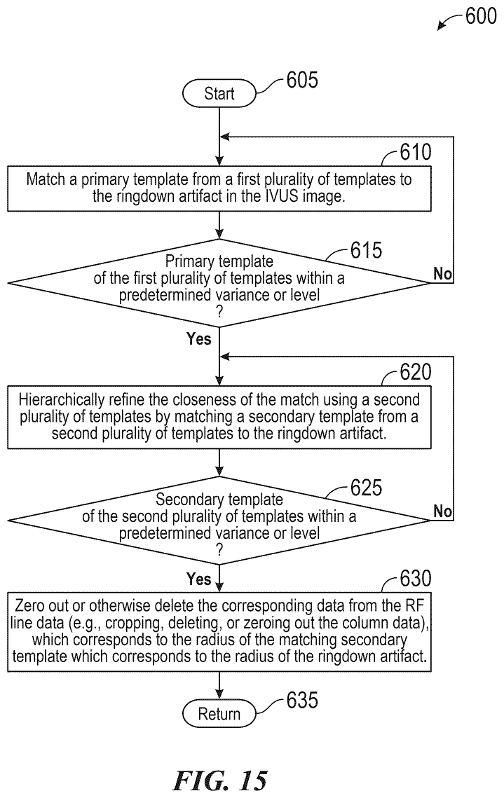

The method 500 begins, start step 505, with the IVUS console 150 receiving the RF line data provided using the IVUS catheter 200. As an option, the IVUS console 150 may remove any ringdown artifact from the ultrasound image, step 510, described in greater detail below with reference to FIG. 15. In step 515, the processor 145 of the IVUS console 150 then converts the RF line data to Cartesian space and displays the resulting ultrasound image (with or without the ringdown artifact) on the image output display 155, and receives a user selection for generation of the vessel contour trace (e.g., autocompletion, autocorrection, or simultaneous suggestion). (It should be noted that if the user does not want the vessel contour trace generated for the currently displayed image, the user may simply select a different image for vessel contour tracing.) When the user has selected autocorrection and has provided an initial, manual vessel contour trace, step 520, the user-provided manual trace will be utilized as the initial vessel contour trace and displayed on the image output display 155, step 525, and the method proceeds to step 555. When the user has selected autocompletion and has provided initial key nodes 420, step 530, the user-provided key nodes 420 will be utilized as accurate or true key nodes 420 in the completed vessel contour trace and displayed on the image output display 155, step 535, and the method proceeds to step 540. By default, if the user has not selected the autocorrection or autocompletion modes, the method proceeds with the mode (and/or its variant, simultaneous suggestion), and also proceeds to step 540.

Using the trained AI processor, trained CNN or trained AI transformer, and using the RF line data, the processor 145 of the IVUS console 150 generates a segmentation of the image to identify the vessel of interest for the vessel contour tracing, step 540. The segmentation process and the training methodology is described in greater detail below with reference to FIGS. 16A and 16B. The processor 145 of the IVUS console 150 then generates an initial vessel contour trace (e.g., from the segmentation result), step 545, and converts the initial vessel contour trace, from RF line data, into Cartesian space (e.g., Cartesian coordinate system). The initial vessel contour trace, either generated from the segmentation step 545 or as provided by the user in step 520, together with any user-provided key nodes 420 provided in steps 530 and 535, are then utilized to initialize the active contour modelling process of step 555.

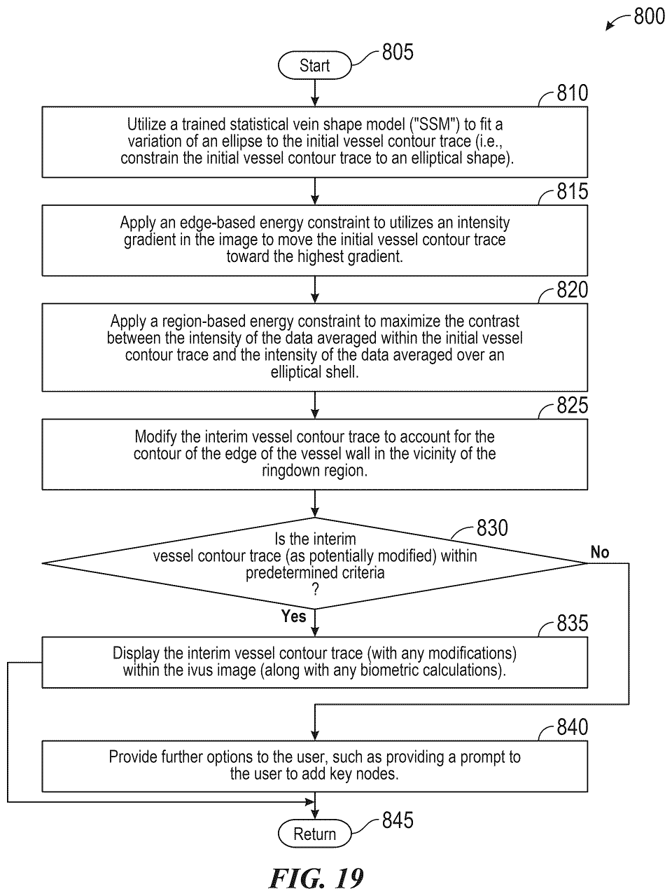

Under the constraints of a trained SSM criteria in step 560, and using an active contour modelling process (e.g., a “snake”), both described in greater detail below with reference to FIG. 19, the processor 145 of the IVUS console 150 generates (or regenerates) a vessel contour trace, step 555. The processor 145 of the IVUS console 150 then provides additional vessel contour trace post-processing with respect to the IVUS catheter 200 location, step 565, also described in greater detail below with reference to FIG. 19, such as by modifying the vessel contour trace in the region between the IVUS catheter and the vessel wall. When the generated vessel contour trace meets or exceeds accuracy criteria in step 570, the processor 145 of the IVUS console 150 displays the generated complete vessel contour trace with the user-interactable (selectable and moveable) key nodes 420 on the image output display 155, step 575. When the user modifies the generated vessel contour trace in step 585, such as by selecting and moving one or more of the various key nodes 420 or by adding more key nodes 420, the method returns to step 555 and iterates. When the generated vessel contour trace does not meet or exceed the accuracy criteria in step 570, the processor 145 of the IVUS console 150 does not display the generated complete vessel contour trace on the image output display 155, and instead provides various options to the user, step 580, such as user prompts to select or input various key nodes 420, and the method iterates, returning to step 535 to receive user input and generate a new vessel contour trace. When the user does not modify the generated vessel contour trace in step 585, the processor 145 of the IVUS console 150 generates and displays one or more selected biometrics, such as effective vein diameter and other biometrics described herein, step 590, and the method may end, return step 595. If there are additional ultrasound images for vessel contour tracing, the user may select the corresponding image and repeat the process, as necessary or desirable.

FIG. 15 is a flow diagram illustrating a computing system implemented method 600 to remove a ringdown artifact in an ultrasound image provided using the IVUS catheter 200 in accordance with embodiments of the present technology. As mentioned above, the IVUS console 150 (having and using the processor 145, the memory circuit 185, and the image output display 155) provides the computing system which implements the method 600 to remove a ringdown artifact in an ultrasound image provided using the IVUS catheter 200. Beginning with start step 605, the processor 145 matches a primary template from a first plurality of templates, to the ringdown artifact 410 in the IVUS image, step 610. The first plurality of templates are generally circular templates, with each template of the first plurality of templates having a different, predetermined and known radius. When a primary template of the first plurality of templates is not within a predetermined variance or level, step 615, the method iterates, returning to step 610 to match a next primary template, from the first plurality of templates, to the ringdown artifact 410 in the IVUS image. When a primary template of the first plurality of templates is within a predetermined variance or level in step 615 (e.g., a 3 mm template) the processor 145 hierarchically refines the closeness of the match using a second plurality of templates, with the processor 145 matching a secondary template from the second plurality of templates to the ringdown artifact 410 in the IVUS image, step 620. The second plurality of templates are also generally circular templates, with each secondary template of the second plurality of templates also having a different, predetermined and known radius (e.g., 2.8 mm, 2.9 mm, 3.0 mm, 3.1 mm, 3.2 mm). The templates from the first and second pluralities of templates differ insofar as the templates from the first plurality of templates are coarser-grained, so are referred to as “primary” templates in the hierarchical matching, while the templates from the second plurality of templates are finer-grained, so are referred to as “secondary” templates in the hierarchical matching. It should be noted that the templates of the first and second pluralities of templates may have additional shapes besides being generally circular, such as to remove artifacts having different shapes, and all such variations are considered equivalent and within the scope of the disclosure.

When a secondary template of the second plurality of templates is not within a predetermined variance or level, step 625, the method iterates, returning to step 620 to match a next secondary template, from the second plurality of templates, to the ringdown artifact 410 in the IVUS image. When a secondary template of the second plurality of templates is within a predetermined variance or level in step 625 (e.g., a 3.1 mm template), the processor 145 zeros out or otherwise deletes the corresponding data from the RF line data corresponding to the IVUS image, step 630, such as cropping, deleting or zeroing out the column data 276 illustrated in FIG. 8, which corresponds to the radius of the matching secondary template which, in turn, corresponds to the radius of the ringdown artifact 410. Following the cropping, deletion or zeroing out of the corresponding column data 276 of the RF line data, the method may end, return step 635. This ringdown removal can be repeated for each frame of RF line data for each corresponding IVUS image, along with any averaging or temporal smoothing to account for further variations. Alternatively, the cropping, deletion or zeroing out of the corresponding column data 276 of the RF line data may be done for any or all of the frames of RF line data for any or all of the relevant, corresponding IVUS images.

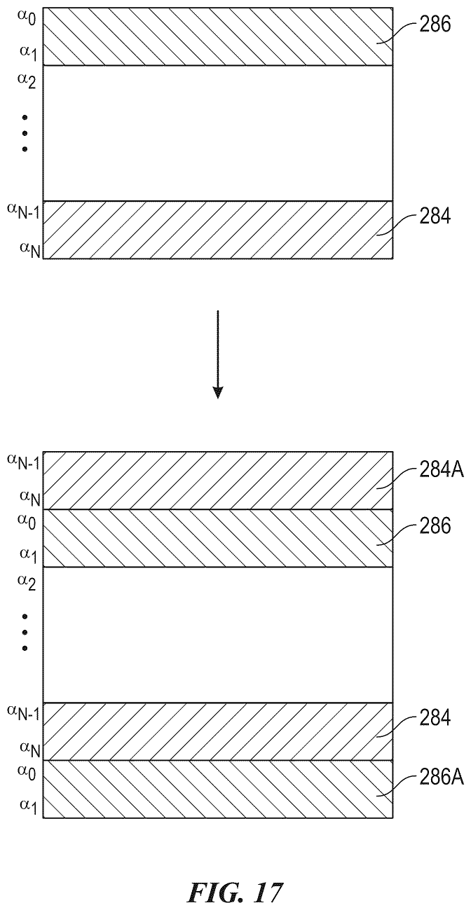

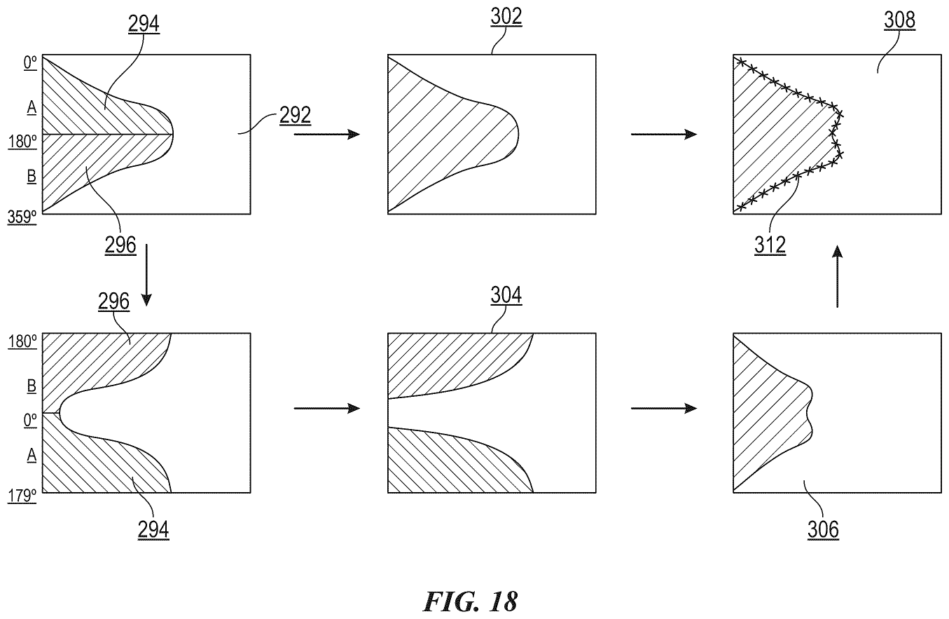

FIGS. 16A and 16B are flow diagrams illustrating a computing system implemented segmentation method 700 to generate an initial vessel contour trace in accordance with embodiments of the present technology. FIG. 17 is a diagram illustrating a frame or matrix of digital ultrasound signal data (RF line data) which has been padded with additional data in accordance with embodiments of the present technology. FIG. 18 is a diagram illustrating a RF line data which has been rotated for double-inference during segmentation in accordance with embodiments of the present technology. As mentioned above, the IVUS console 150 (having and using the processor 145, the memory circuit 185, and the image output display 155) provides the computing system which implements the segmentation method 700 to generate an initial vessel contour trace. Various software platforms may be utilized to implement and train the segmentation process performed by the processor 145, including without limitation YOLO v.8 (You Only Look Once) from Ultralytics, Inc. (available at www.ultralytics.com) (5001 Judicial Way, Frederick, MD 21703 USA); or vision transformers with CNNs or other methods with convolutional neural networks (e.g., Mask-Dino, SAM), hybrid approaches combining vision transformers with conventional convolutional neural networks, or methods based on convolutional neural networks (Mask RCNN, SegNet) etc. This disclosure is not limited to deep learning methods, however, and other methods may be utilized within the scope of this disclosure, including binary or adaptive thresholding, region growing, graph-cuts, watershed, and other segmentation methods, for example.

Referring to FIGS. 16A-18, the segmentation method 700 begins, start step 705, using a trained AI processor, trained CNN or trained AI transformer, for example, with generating a plurality of RF line data channels, namely, a positionally-weighted RF line data channel, step 710, and a depth normalization RF line data channel, step 715, while the original RF line data is also retained as a third RF line data channel. As a vessel wall appears in the near field of an IVUS image, for the positionally-weighted RF line data channel, the near field RF line data 278 (illustrated as columns of RF line data in FIG. 8) is given a higher weighting compared to the remaining, far field RF line data 282 (also illustrated as columns of RF line data in FIG. 8). This also reduces any time gain compensation which may have been provided to the far field data. In some embodiments, the near field RF line data 278 is assigned a digital “1” weighting, so that the near field RF line data 278 is included in the positionally-weighted RF line data channel, while the far field RF line data 282 is assigned a digital “0” weighting, so that the far field RF line data 282 is not included in the positionally-weighted RF line data channel.

As described above, in the frame or matrix 248, each column 246 represents a different depth in the IVUS image. For each column Cᵢ, a mean value is determined for all of the intensity data (pixel data) in that column Cᵢ, for all columns 246 in the frame or matrix 248. For the depth normalization RF line data channel, each intensity value is then divided by the mean intensity value for the selected depth/column: normalized I_{Ci}=I_{Ci}/mean I_{Ci} (Equation 1), for example. Other methods of generating a depth normalization RF line data channel, such as using an average of the minimum and maximum of each column/depth, or using a standard deviation or other statistical measure of each column depth, for example, are also considered equivalent and within the scope of the disclosure. The original RF line data for the IVUS image is then utilized as a third RF line data channel.

The three RF line data channels (the positionally-weighted RF line data channel, the depth normalization RF line data channel, and the original RF line data channel) are combined together (e.g., added) into a combined RF line data channel as the input into the balance of the segmentation process 700, step 720 (e.g., the corresponding intensity values at each angle α 274 and at each depth 246) from each RF line data channel, are added together to create a single intensity value at each angle α 274 and at each depth 246 to form the combined RF line data channel.

Depending upon how the segmentation method 700 is implemented, the combined RF line data channel may need to be padded (with corresponding image resizing), step 725, such as to be divisible by 32, for example. In some embodiments, the data padding takes note of the fact that the first line and the last line of the RF line data are adjacent or connected to each other during the scan conversion (e.g., 359° is next to 0°), so that continuity of the segmentation space is maintained by mirroring the RF line data with adjacent RF line data. One method of data padding is illustrated in FIG. 17, using adjacent or surrounding rows of data, such that one or more of the lower rows of data 284 are copied to form upper rows 284A and one or more of the upper rows of data 286 arc copied to form lower rows 286A. For example, the data at angles 358° and 359° are copied to wrap around and be adjacent to 0°, and the data at angles 0° and 1° are copied to wrap around and be adjacent to 359°. Alternatively, the rows and columns of the combined RF line data channel may be padded with zeros on the right and bottom edges, as needed, also for example.

One or more second sets of IVUS image RF line data is then generated, step 730, such as with a 180° image orientation shift (e.g., the segmentation method flips or rotates the image around the IVUS catheter 200 longitudinal axis 85 by wrapping the RF data lines at the selected or desired orientation shift). As illustrated in FIG. 18, for the RF line data frame or matrix 292, the RF line data 296 (from 180° to 359°) has been wrapped or shifted 180° to form the RF line data frame or matrix 298. For each IVUS image, both the first and second sets of RF line data, the original and the shifted, are utilized in both segmentation training and segmentation prediction/inference, so that both images are utilized in the training and prediction processes, providing a dual inference. In step 735, a first RF line data frame or matrix is provided as input into the trained AI processor, trained CNN or trained AI transformer to obtain a first segmentation prediction result (illustrated as RF line data frame or matrix 302 in FIG. 18), and in step 740, a second RF line data frame or matrix (which is the image orientation-shifted version of the first RF line data frame or matrix) is also provided as input into the trained AI processor, trained CNN or trained AI transformer to obtain a second segmentation prediction result (illustrated as RF line data frame or matrix 304 in FIG. 18). To be commensurate with the first segmentation prediction result, the second segmentation prediction result is then shifted back (e.g., by 180°) to have the same image orientation as the first segmentation prediction result, resulting in an orientation-shifted second segmentation prediction result, illustrated as RF line data frame or matrix 306, step 745. In step 750, the first segmentation prediction result is then combined or merged with the orientation-shifted second segmentation prediction result, such as by using a logical AND operation or a logical OR operation (e.g., ANDing or ORing each corresponding pixel of the first segmentation prediction result with the orientation-shifted second segmentation prediction result), to generate a combined segmentation prediction result from the dual inference process, such as the combined segmentation prediction result illustrated as RF line data frame or matrix 308 in FIG. 18).

This double or dual inference process for segmentation has many additional benefits and improves accuracy, such as be removing or decreasing over-segmentation, under-segmentation, and noise. For example, the double inference process tends to reduce or eliminate over-segmentation, in which the selected segment overlaps into the vessel wall, and under-segmentation, in which the selected segment is excessively far away from the vessel wall. It should also be noted that in addition to creating a second RF line data frame or matrix (which has been shifted 180° from the first RF line data frame or matrix), additional RF line data frames or matrices may also be generated and utilized, such as RF line data frames or matrices shifted by 90° and 270°, for example.

This segmentation process may be performed for any region of interest in the IVUS image, in addition to veins, such as arteries, collateral veins, arteries with stents, veins with stents, and veins or arteries with or without various occlusions such as blood clots. These segmentations may then be transformed into “masks”, such as to improve the speed of processing, such as creating a “multi-class” mask for segmenting veins, arteries, and collateral veins, for example, or a single binary mask, such as for veins and veins with blood clots, also for example.

The segmentation method may also include optional post-processing, including de-padding the combined segmentation prediction result (e.g., removing padded data from the RF line data frame or matrix) and re-sizing the IVUS image, step 755, and filtering the combined segmentation prediction result, step 760, such as filtering out smaller objects and only retaining the largest or maximum segmentation indicative of the vessel of interest, for example.

The processor 145 then extracts the “edge” data from the combined segmentation prediction result to generate an initial vessel contour trace, step 765, illustrated as initial vessel contour trace 312 in FIG. 18, and converts the initial vessel contour trace from RF line data to the Cartesian coordinate system (Cartesian space), if needed for the active contour module, step 770. The initial vessel contour trace is then utilized to initialize the active contour modelling process (of step 555), and the segmentation method may end, return step 775.

FIG. 19 is a flow diagram illustrating a computing system implemented method for active contouring and statistical shape modelling to generate and display the user-interactive vessel contour trace in an ultrasound image provided using the IVUS catheter in accordance with embodiments of the present technology. As mentioned above, the IVUS console 150 (having and using the processor 145, the memory circuit 185, and the image output display 155) provides the computing system which implements the method 800 for active contouring and statistical shape modelling to generate and display the user-interactive vessel contour trace in an ultrasound image provided using the IVUS catheter 200. Also as mentioned above, the active contour modelling process refines the initial vessel contour trace by finding the precise vein edges using gradient, brightness changes, as well as shape constrains to iteratively improve the vessel contour trace from the initial vessel contour trace provided by the segmentation module, generating an interim vessel contour trace. Beginning with start step 805, the processor 145 uses an active contour model (e.g., a parameter or parametric snake) to iteratively morph or drive the initial vessel contour trace toward the true vessel wall edge by optimizing an energy function having at least three parameters or constraints: (1) a statistical shape energy or constraint; (2) an edge-based energy or constraint; and (3) a region-based energy or constraint. In step 810, a trained SSM fits a variation of an ellipse to the initial vessel contour trace (e.g., constrains the initial vessel contour trace to an elliptical shape), which regularizes the initial vessel contour trace to a statistically more typical elliptical vein shape, as described in greater detail below with reference to FIGS. 20A and 20B. In step 815, the edge-based energy constraint utilizes the intensity gradient in the image to move the initial vessel contour trace toward the highest gradient, where the black (zero) values of the IVUS image become white (one) values indicative of the edge of the vessel wall. In step 820, the region-based energy constraint maximizes the contrast between the intensity of the data averaged within the initial vessel contour trace, and the intensity of the data averaged over the elliptical shell. It should be noted that these parameters or constraints of steps 810, 815, and 820 may be applied concurrently or sequentially by the processor 145. The output of the active contour model generates an interim vessel contour trace which, as an option, may be smoothed further, such as by using spline interpolation. The active contour model which may be utilized may have other or additional settings or controls as well, including the number of control points, regularization factors or weightings which may be applied to each of the shape, edge, and region constraints (or energies), and the maximum number of iterations which may be allowed during the iterative optimization of the vessel contour trace, for example. Alternatively, the optimization can finish when the sum of energies is below a predetermined threshold, also for example.

The processor 145 will also modify the interim vessel contour trace to account for the contour of the edge of the vessel wall in the vicinity of the ringdown region, step 825. In many IVUS images, the IVUS catheter 200 is often abutting or very close to the vessel wall, with the ringdown artifact 410 obscuring portions of the vessel wall, such as illustrated in FIGS. 10-13. As the radius (Rc) of the IVUS catheter 200 is known, and the radius of the ringdown artifact 410 has been determined, and given that the IVUS catheter 200 is always the center point of the IVUS images, the processor 145 calculates the distance between each point on the interim vessel contour trace (Xp) and the longitudinal center of the IVUS catheter 200 (Xc). If any calculated distance is less than or equal to the radius of the IVUS catheter 200 (|Xp−Xc|≤Rc), that portion of the interim vessel contour trace has crossed the IVUS catheter 200 and, in accordance with the representative embodiments, is moved to a distance which is greater than the radius of the IVUS catheter 200, typically between the outer edge of the IVUS catheter 200 and the ringdown artifact 410, for example. Using the SSM, when the interim vessel contour trace (as potentially modified in step 825) is within predetermined criteria, such as predetermined quality metrics, step 830, the interim vessel contour trace (with any modifications) is displayed within the IVUS image (along with any biometric calculations, as described above), step 835. When the interim vessel contour trace (as potentially modified in step 825) is not within the predetermined criteria, such as the predetermined quality metrics in step 830, the processor 145 does not display the interim vessel contour trace and instead provides further options to the user, step 840, as described above, such as providing a prompt to the user to add key nodes 420 (which will trigger auto-tracing, as described above), also for example. Following steps 835 and 840, the vessel contour trace method may end (e.g., return step 845).

FIGS. 20A and 20B are flow diagrams illustrating a computing system implemented method 900 for statistical shape modelling to generate and display the user-interactive vessel contour trace in an ultrasound image provided using the IVUS catheter in accordance with embodiments of the present technology. The SSM modelling is used at two different stages in the vessel contour tracing methodology. First, the SSM model is applied to the initial vessel contour trace provided by the segmentation module, in step 805, described above. This additional step reduces number of steps required by the active contouring (e.g., snake) to find the optimal solution. Second, SSM modelling is used on the interim vessel contour trace, and fitting coefficients such as weightings are used to calculate quality metrics. Based on these quality metrics, the SSM modelling determines whether the interim vessel contour trace (as potentially modified in step 820) will be shown to the user in step 825. The statistical shape modelling illustrated in FIGS. 20A and 20B includes three distinct phases, the training phase (steps 910, 915, and 920), the inference phase (steps 925, 930, and 935), and the quality metrics phase (steps 940, 945, 950, and 955).

Statistical Shape Models (SSMs) can express a range of expected, evidence-based variation on top of a mean shape (e.g., an elliptical trace or contour) derived from a cohort or population. The purpose of utilizing the SSM for the vessel contour trace is to assure that the generated vessel contour trace should vary only in ways seen in the training set of labelled examples, such as from the labelled IVUS images utilized in the segmentation process, e.g., the plurality of ultrasound images with their accurate, professional, marked (e.g., manually labelled) segmentations providing accurate contour traces for use as the SSM training data set. Each shape or contour is represented by a set of two-dimensional (“2D”) landmarks (e.g., samples) in the IVUS image coordinates. A first step is to align the training set, e.g., remove any variations that are caused by global shape transformations. The aligned training set forms a cloud in the 2×N dimensional space, where “N” is the number of shape points, and can be considered to be samples from a probability density function. In the simplest formulation, the cloud may be approximated with a Gaussian, for example. Principal component analysis (“PCA”) is utilized to select the main axes of the cloud, and model only the first few, which account for the majority of the variations.

The process of training the SSM modelling involves three steps, as illustrated in FIGS. 20A and 20B. In this process, each shape or contour may be represented as a variance (e.g., x₁, x₂, x₃, . . . x_{N}) from a mean shape or contour (e.g., x̅), so that a plurality of shapes or contours may be represented by the mean and variance (e.g., x₁-x̅, x₂-x̅, x₃-x̅, . . . x_{N}-x̅). Following start step 905, the first step is to sample a fixed number of points from each shape or contour of the training data set and geometrically align the training data (by adjusting or removing 2D translation, scale and rotations) and scale the shape landmarks to a normalized space, such as [−1, 1], step 910. The second step is to compute a mean shape or contour (e.g., x̅), step 915, where each landmark is the average of the corresponding landmarks in the training data. The third step is to apply PCA to extract the main modes of shape or contour variation by calculating the eigenvalues of the covariance matrix (Cov=M·M^{T}) of all of the training data, step 920, such as by applying singular value decomposition to generate a matrix of eigenvalues (λ₁, λ₂, λ₃, λ₄, . . . λ_{N}), a corresponding matrix of variations (e₁, e₂, e₃, e₄, . . . e_{N}, with each “e” being a vector), and corresponding weightings (w₁, w₂, w₃, w₄, . . . w_{N}), with the highest eigenvalues corresponding to or indicative of the principal variations. As a result, the SSM model may be represented as a mean with weighted (“wᵢ”) variations (Equation 2):

$$x = \overline{x} + {\sum }_{i = 0}^{n} {w}_{i} {e}_{i} .$$

In some embodiments, each shape or contour is represented by “N” 2D landmarks, such as with N=128 (although the number of landmarks may vary), and the number of principal variations is limited or trimmed to retain 94%-98% of the total variations, as experimentally determined, for example.

The process of shape fitting (or inference) for the initial or interim vessel contour trace using the SSM modelling involves three steps, also as illustrated in FIGS. 20A and 20B. First, in step 925, initial or interim vessel contour trace is input and sampled, with the landmarks of the initial or interim vessel contour trace (as the input shape) being scaled to match the coordinate space of the mean shape (e.g., [−1, +1], [0, 1]). Second, in step 930, the scaled initial or interim vessel contour trace is aligned with the mean shape (x̅, from step 915), such as by using a Procrustes Analysis, also for example, which provides isomorphic scaling, translation, and rotation to find the “best” fit between the scaled initial or interim vessel contour trace and the mean shape. Third, in step 935, the SSM model is applied to the aligned shape (“x”) to obtain the weightings or other fitting coefficients, such as by using a cosine similarity, (e.g., obtaining the weightings wᵢ for the scaled initial or interim vessel contour trace), such as by using (Equation 3):

$${w}_{i} = {{e}_{i} ( x - \overline{x} )}^{T} .$$

These coefficients indicate the statistical distribution of the input shape (e.g., whether it follows a normal distribution), and are also utilized in the quality metrics. Typically, the fitting coefficients are constrained within [−3, +3] standard deviations.

Lastly, quality metrics may be determined, step 940, with the weightings wi for the scaled initial or interim vessel contour trace compared to a predetermined amount or level (e.g., within 3 standard deviations) of the corresponding eigenvalues (λ₁, λ₂, λ₃, λ₄, . . . ), which provides about a 98% confidence level. More particularly, in step 940, the processor 145 determines whether each weighting is within a predetermined amount or level, such as within three standard deviations of the corresponding eigenvalue, where the three standard deviations is represented as \sqrt{3}, using Equation 4:

$$\vert {w}_{i} \vert \leq \sqrt{3} \vert λ i \vert .$$

When the weighting is within (or equal to) three standard deviations of the corresponding eigenvalue in step 940, that shape variation is acceptable and may be utilized to determine the final or complete vessel contour trace, which may be utilized and displayed, step 945. When the weighting is not within three standard deviations of the corresponding eigenvalue in step 940, that shape variation is not acceptable (and is considered statistically wrong) and is not utilized to determine the final or complete vessel contour trace, step 950. Following steps 945 and 950, when there are any additional weightings for comparison, step 955, the method iterates, returning to step 940. When there are no further weightings to evaluate in step 955, the method may end, return step 960. As indicated above, the SSM model is used on the final output from the contour tracing, and the weightings or other fitting coefficients are used to calculate quality metrics, which determines whether the contour trace is displayed to the user.

Alternatively, in another representative embodiment, the quality metrics are calculated using Equation 5, where “z” is a set of non-zero fitting coefficients, where coefficients above lower and upper thresholds are set to 0, and “n” is the number of coefficients (Equation 5):

$$C = \frac{{\sum }_{i = 0}^{n} {z}_{i}}{n} .$$

Alternatively, confidence metrics could be calculated as a weighted sum from all coefficients, or by setting a threshold only on a selected single coefficient, for example.

As mentioned above, the various representative embodiments provide an apparatus, method and system for vessel contour tracing in intravascular ultrasound imaging. The representative IVUS apparatus, method, and system embodiments provide for assisting the end-user to obtain accurate measurements of certain biomarkers, such as vein/vessel area, minimum diameter, maximum diameter, average diameter and effective diameter, for example. Such an IVUS apparatus, method, and system uses a single or a plurality of IVUS images or a short IVUS sequence as input, and on each IVUS input, the representative IVUS apparatus, method, and system embodiments trace the vein wall and display the traced vein shape to the end-user (provided the vessel contour trace is considered sufficiently accurate). The representative IVUS apparatus, method, and system embodiments allow the user to accept the traced vein shape, edit it manually, or ignore it and re-trace the vein manually. Once the auto-tracing is completed, the representative IVUS apparatus, method, and system embodiments are able to calculate the biomarker measurements, among other features. The representative IVUS apparatus, method, and system embodiments provide reliable and reproducible vessel contour tracing, including avoiding various or typical user mistakes, such as the erroneous inclusion of the ringdown artifact within the region of the vessel contour trace.

### IV. Selected Embodiments of Blood Flow Detection Systems

FIG. 21 is a first ultrasound image 430 of a vein 415 with a ringdown artifact 410, in accordance with embodiments of the present technology. In the first ultrasound image 430, the vein 415 appears to be largely black (limited to no brightness) with some visible graininess, bounded by the venous wall 435, which appears much brighter. Various ultrasound images are also known to include “speckle”, which is a random granular texture that may obscure anatomy in ultrasound images and is usually described as a type of noise. Speckle may be created by a complex interference of ultrasound echoes made by reflectors spaced closer together than the ultrasound system's resolution limit, for example, such as from blood cells. As used herein, speckle simply may be considered to be one or more pixels (or grains) having a selected brightness in the ultrasound image, such as one or more pixels having a selected brightness in the ultrasound image within a blood vessel (e.g., vein 415) in a region in which an ultrasound reflection from other tissue would not be expected. As blood vessels typically appear as a largely black region 455 bounded by a vessel wall (e.g., venous wall 435) having a much higher brightness in an ultrasound B-mode image, the speckle appearance within the blood vessel within the ultrasound image may be considered to be indicative of blood cells, and such speckle it utilized to detect, assess and image blood flow in accordance with embodiments of the present technology.

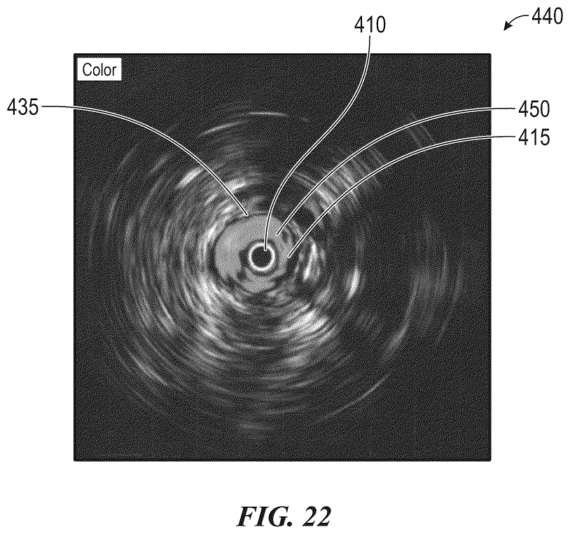

FIG. 22 is a second ultrasound image illustrating the first ultrasound image of FIG. 21, and further illustrating an overlaid, representative image (in red and yellow) of blood flow 450 in accordance with embodiments of the present technology. Such a blood flow image 450 is generated using speckle tracking or tracing in successive frames (805, 810) of RF line data, as described in greater detail below, and is overlaid or merged with the B-mode image to generate the combined, second ultrasound image 440. In various representative embodiments, different colors (such as red and yellow) may be utilized to indicate the speed or velocity of blood flow, for example.

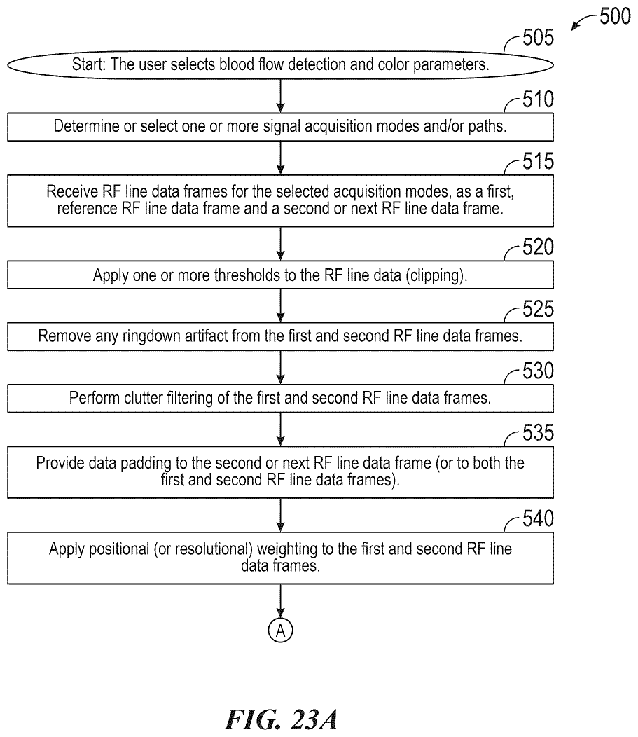

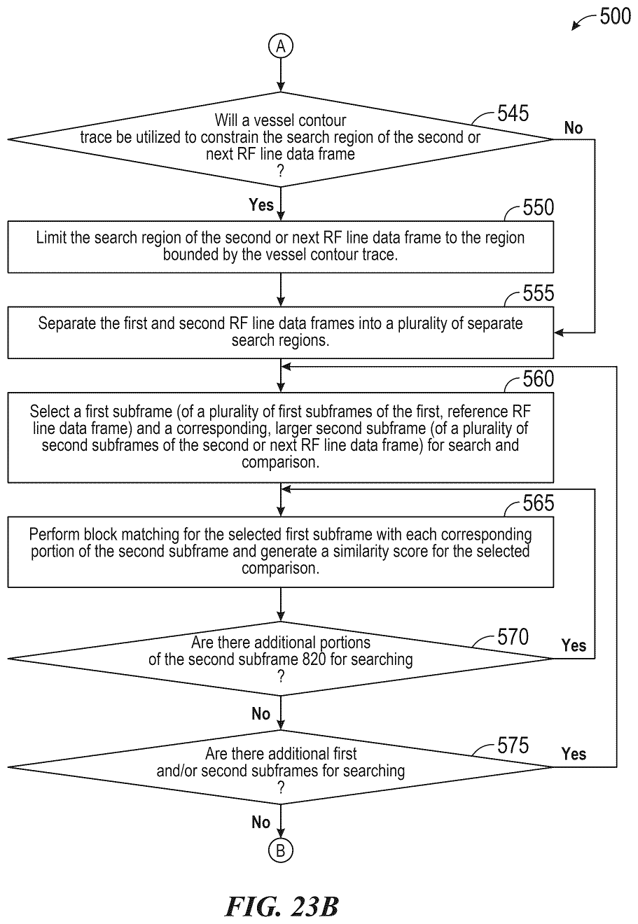

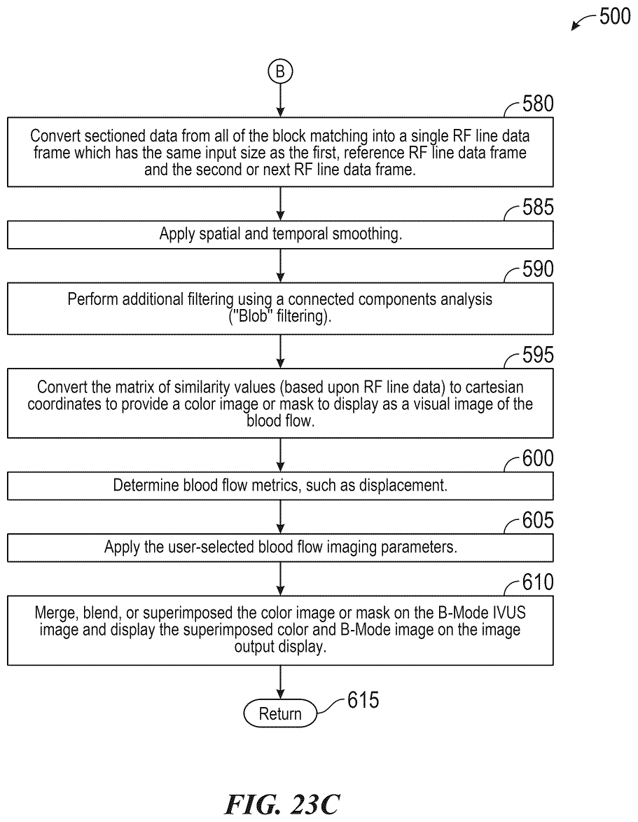

In some embodiments, a vessel contour trace 405 may also be overlaid both with the ultrasound image 400 of a vein 415 and also the blood flow image (not separately illustrated). In some embodiments discussed in greater detail below with reference to FIGS. 23A-23C, as an option, the vessel contour trace 405 may be utilized to provide a searching boundary for the block or subframe matching between successive frames (805, 810) which are utilized in the formation of one or more blood flow images in accordance with embodiments of the present technology.

FIGS. 23A, 23B, and 23C are flow diagrams illustrating a computing system implemented method 500 to generate and display a user-interactive blood flow image 450 in an ultrasound image 440 provided using the IVUS catheter 200 in accordance with embodiments of the present technology. The IVUS console 150 (having and using the processor 145, the memory circuit 185, and the image output display 155) provides the computing system that implements the method 500 to generate and display a user-interactive blood flow image 450 in an ultrasound image 440 provided using the IVUS catheter 200. In some embodiments, the method described in greater detail above with reference to FIG. 15 can be used to remove a ringdown artifact in the ultrasound image 440 provided using the IVUS catheter 200.

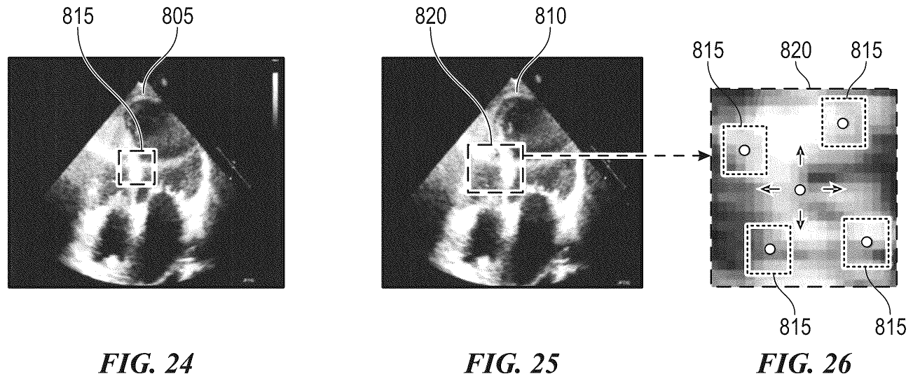

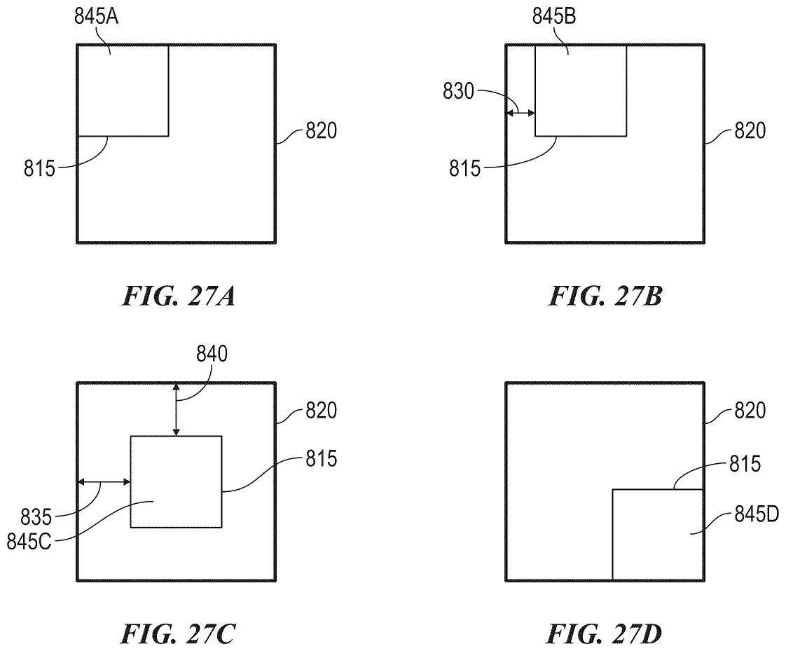

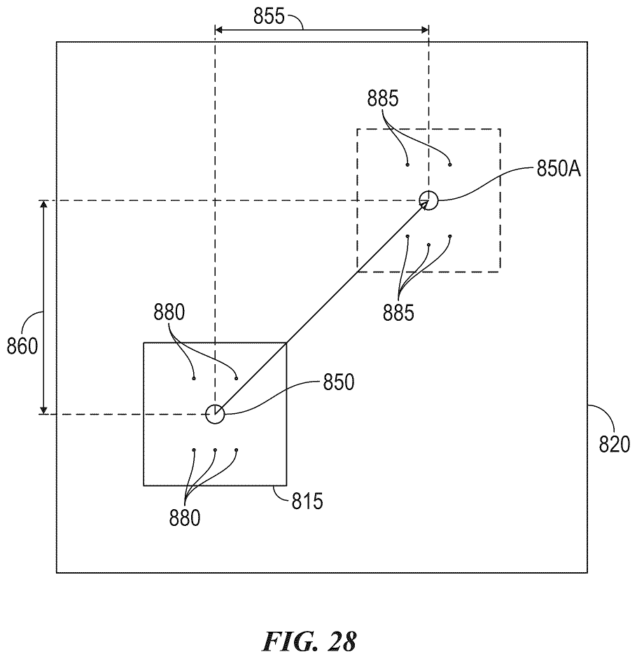

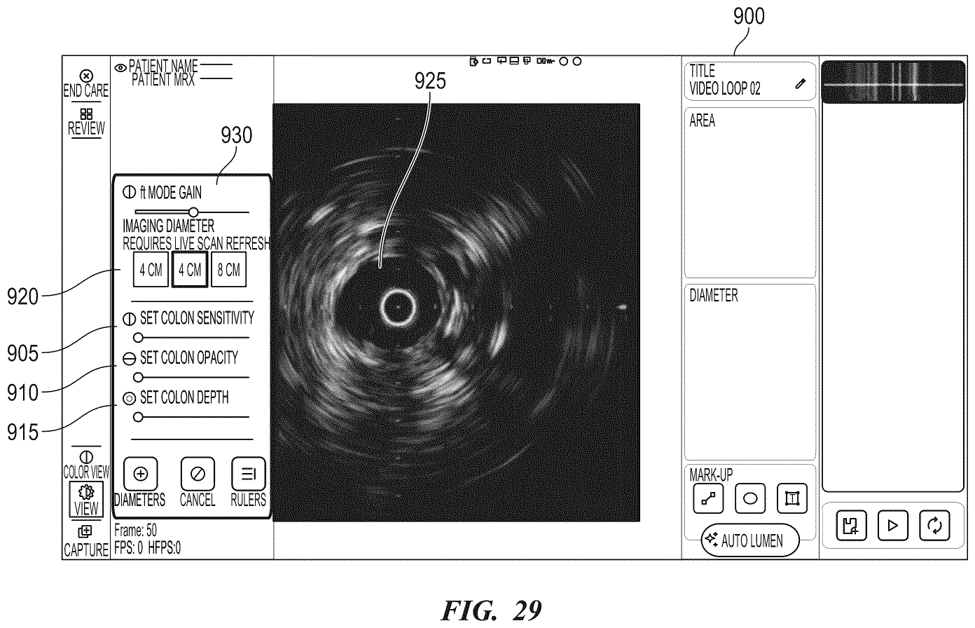

FIG. 24 is a first ultrasound RF line data frame 805, utilized as a reference frame, illustrating selection of a first subframe (or block) for block 815 or subframe searching and/or matching in accordance with embodiments of the present technology. FIG. 25 is a second (or next) ultrasound RF line data frame 810 illustrating selection of a second subframe (or block) 820 for block or subframe searching and/or matching in accordance with embodiments of the present technology. FIG. 26 is a diagram illustrating block or subframe searching and/or matching of the first subframe (or block) 815 within the second subframe (or block) in accordance with embodiments of the present technology. FIGS. 27A, 27B, 27C, and 27D are a sequence of diagrams illustrating block or subframe searching and/or matching of the first subframe (or block) 815 within a sliding window of the second subframe (or block) 820 in accordance with embodiments of the present technology. FIG. 28 is a diagram illustrating the selection of a region matching the first subframe (or block) 815 within the second subframe (or block) 820 and distance measurement for block or subframe searching and/or matching in accordance with embodiments of the present technology. FIG. 29 is an IVUS console display 155 screen shot or image illustrating a graphical user interface (“GUI”) 900 having a fourth ultrasound image 925 of a vein 415 and illustrating an overlaid, representative user-interactive blood flow detection and color selection interface 930 in accordance with embodiments of the present technology.

As described above, speckle is a very common phenomenon in ultrasound imaging and refers to the grainy appearance, like tiny bright dots or noise, that is caused by the interaction of ultrasound waves with the tissues being scanned. It is assumed that as the ultrasound waves reflect from the same tissue and blood cells, they show similar speckle structures, and the speckle tracking of this disclosure aims to track these features. As described in FIG. 23A-23C, the method 500 works on any two successive or consecutive frames of RF line data, a first reference RF line data frame 805, and a second or next search and comparison RF line data frame 810 for search. As the method 500 continues, the second RF line data frame 810 will become the next reference RF line data frame 805, and a next, third, frame (not separately illustrated) will become the next search and comparison RF line data frame 810. Once the first two RF line data frames 805, 810 are obtained, the reference RF line data frame 805 is divided into smaller region of interest sections, referred to as first subframes 815 (or target regions) and then each first subframe 815 is searched in the next RF line data frame 810 within (slightly) larger sections, referred to as second subframes 820, to find the best match and obtain distance calculations. In a first representative embodiment, each first subframe 815 is searched in one second subframe 820 or in a limited number of second subframes 820 in the region or vicinity within the second or next search and comparison RF line data frame 810 into where it is likely that the slow blood represented in the first subframe 815 might have flowed. In a second representative embodiment, each first subframe 815 is searched in all second subframes 820 within the second or next search and comparison RF line data frame 810.

The first subframe 815 has special speckle characteristics, and it is searched in the second subframe 820 by using a block matching technique, for example. As indicated previously, the interior of a vein appears black in an ultrasound image, with a significant portion of the pixels in the ultrasound image effectively having a zero (or close to zero) value, and with the speckle appearing as pixels having non-zero values, such as illustrated with pixels 880 in the first subframe 815 illustrated in FIG. 28, for example. The closest or best matching section 875 of the second subframe 820 is selected, and displacement calculations (displacements 855, 860) are made to detect motion. It should be noted that the matching section 875 has a similar, but not exact, arrangement of non-zero pixels 885, indicating speckle movement (blood flow) between the successive first reference RF line data frame 805 and the second or next RF line data frame 810. If the first subframe 815 in the first reference RF line data frame 805 is found at exactly the same location in the second or next RF line data frame 810, no motion is likely to have been detected, but is more likely to be an artifact or noise, as some variation would be expected from flow of blood. This process of selecting and comparing a first subframe 815 with a second subframe 820 is performed for all of the first subframes 815 of the first reference RF line data frame 805 having non-zero pixel values (or non-zero pixel values greater than a predetermined threshold). In some embodiments, this process of selecting and comparing a first subframe 815 with a second subframe 820 may be performed in parallel for all first subframes 815 of the first reference RF line data frame 805, with each search and comparison of a selected first subframe 815 with a selected second subframe 820 assigned to a separate GPU core 148, for example.

As the closest or best matching section 875 recedes across successive RF line data frames, the speed of blood flow may be determined based upon the degree or amount of displacement, and the range of speeds of blood flow may be displayed using the color spectrum, such as the color yellow for a first speed and the color red for a second, faster speed. The method 500 is repeated for all new arriving frames, by replacing the first reference RF line data frame 805 by the second or next RF line data frame 810 and using the newly arrived RF line data frame as the new search and comparison RF line data frame 810.

Because the blood flow detection method 500 utilizes consecutive echo responses at a subregional ultrasonic element for fast flow, as well as two consecutive ultrasound RF line data frames for slow flow 805, 810, the method 500 eliminates prior art delays as waiting until an entire frame buffer is obtained is not required. Since the method 500 allows the first reference RF line data frame 805 to move in all possible directions, it also does not require perfect parallel blood flow, which is the most limiting assumption of the prior art. Lastly, the speckle tracking approach allows detection of any motion attitudes because it does not assume any motion in advance and explores all nearby locations to find the best match.

The method 500 begins, start step 505, with the user selecting blood flow detection and various color parameters for the blood flow detection, such as color sensitivity, depth and saturation, in addition to other B-mode parameters (such as illustrated in the GUI 900 illustrated in FIG. 29), such as transmit frequency, gain, the line density (e.g., 64, 128), the number of cycles, spatial compounding, apodization, aperture size, transmit focus, etc. In step 510, either automatically or as selected by the user, one or more signal acquisition modes and/or paths are determined or selected. In a first representative embodiment, RF line data is acquired in a dual mode, one for B-mode and another for a blood flow (or Color) mode. For example, in one variation, alternating lines (e.g., every other line, such as all odd numbered lines) of the RF line data frame are utilized for the B-mode signal acquisition and the remaining alternating lines of the RF line data are used for blood flow (or Color) mode (e.g., all even numbered lines). The total number of scan lines could be any combination of number of elements (e.g., 64, 128, 256). Scanlines for each mode can be acquired with different imaging parameters, such as analog gain, transmit frequency, etc. In another variation, RF line data is acquired every second frame for B-mode, and every other frame for blood flow (or Color) mode, respectively. In another variation, RF line data is acquired at a pre-defined ratio of B-mode data to blood flow (or Color) mode data. For instance, when 1-to-3 ratio is used, a first RF line data frame is used for B-mode imaging, and the next three RF line data frames are used for blood flow (or Color) mode imaging, which can provide a higher frame rate to blood flow (or Color) mode and boosting its accuracy. In a second representative embodiment, RF line data is acquired in a single mode, such as B-mode, and modified to optimize the data for blood flow (or Color) mode, thus keeping only one signal path and maximizing the overall frame rate. In step 515, RF line data frames are received for the selected acquisition modes, as a first reference RF line data frame 805 and a second or next RF line data frame 810. As the ultrasound process continues, additional RF line data frames will continue to be received, with any successive RF line data frames being referred to as a first RF line data frame 805 followed by a second or next RF line data frame 810, which may be acquired in any mode, such as in B-mode, blood flow (or Color) mode, or any combination of B-mode and/or blood flow (or Color) mode.

In step 520, as an option the received RF line data for the blood flow (or Color) mode may be clipped, applying one or more thresholds to the RF line data (to both of the first reference RF line data frame 805 and the second or next RF line data frame 810), such as capping the data to less than or equal to a maximum value (e.g., hard, soft, Otsu's thresholding, local adaptive thresholding). For example, using a intensity scale of 0 to 80, values greater than 80 may not provide additional information, and may be zeroed out or replaced with a threshold value.

Additional thresholding may also be performed as an option in step 520 (or later, in post-processing, such as after step 565 or 575), such as to reduce the amount of data that may need to be processed. One way of eliminating unnecessary flow information is to gray-scale values above which flow information is suppressed. It is assumed that flow in the blood vessels is anechoic and vessels are hypoechoic. Any flow arising from pixels that are not anechoic or very hyperechoic are avoided using a single or a plurality of gray-scale value thresholds. In this step, physiological blood flow velocities may be utilized alternatively and the potential motion flow values based on them may be suppressed or enhanced. First, given the fact that the blood moves at a rate of approximately 1.2-4.8 ml/minute in veins in adult human subjects, and if the best matching section 875 is found to have exceeded that upper limit, it is likely to mean that it is only a noise value. Second, the method 500 has not eliminated any similarity values up to this point, but if a similarity metric value is too high for the corresponding section, that may indicate that the best matching section 875 does not represent the first subframe 815 (the target region of interest) anymore. Another way of eliminating non-flow information is to apply locational confidence metric. It is commonly known that SNR of ultrasonic waves reduces as it penetrates further than the signal source that causes more noise interference which resembles blood cell speckles. A spatial confidence threshold would be utilized to aid the low SNR problem in far field. In some embodiments, one or more different upper limits may be applied to cancel the contribution of such potentially erroneous calculations in the final matching decision. The values that are lower than the threshold(s) remain in the matching process.

In step 525, the ringdown artifact 410 is removed, which may be performed in a first representative embodiment as illustrated in FIG. 23A-23C or which may be performed in a second representative embodiment by using an estimated ringdown artifact radius, with all the data samples from the input RF line data frames that are below the ringdown radius being removed, such as column data 276, for both of the first reference RF line data frame 805 and the second or next RF line data frame 810.

Alternatively, as previously indicated, the cropping, deletion or zeroing out of the corresponding column data 276 of the RF line data may be done for any or all of the frames of RF line data for any or all of the relevant, corresponding IVUS images, without any template matching. For example, the ringdown artifact radius may be estimated, and all the data samples from the input RF line data frame that are below the ringdown radius are removed, such as column data 276.

Referring to FIG. 23A, in step 530, clutter filtering is performed, for both of the first reference RF line data frame 805 and the second or next RF line data frame 810. Clutter, including probe motion, reverberation clutter, side lobe clutter, electrical noise clutter, can mask or mimic real movement, hindering the ability to differentiate true speckle motion from background noise. By suppressing clutter, filtering improves the signal-to-noise ratio (SNR), enabling more precise tracking and reducing spurious errors. Furthermore, clutter filtering enhances visualization by providing a clearer image, aiding researchers and clinicians in interpreting the tracked tissue dynamics, but without inadvertently removing valuable information about slow-moving tissues and subtle flow patterns. Optimization of filter parameters will typically balance parameters to maximize SNR and preserve relevant data, and any of several known clutter filters may be utilized equivalently, including high-pass filters (FIR, IIR, Butterworth), adaptive filters (weighted median, clutter rejection with spatial eigenvector filtering), SVD-based, eigen-based, Wavelet and Fourier-based clutter filters, as well as anisotropic diffusion, coherence enhancing non-linear diffusion filters, etc.

In step 535, data padding is provided to the RF line data frame, generally only the second or next RF line data frame 810 (or to both of the first reference RF line data frame 805 and the second or next RF line data frame 810), as described in greater detail above with reference to FIG. 17. Additionally, the reverse of the padding method erases the added lines before Cartesian conversion can be utilized after all operations depicted in FIGS. 23A-23C are completed in order to meet the expected RF frame line data shape.

The speckle tracking of the disclosure uses a typically square or rectangular second subframe 820 as the search space around each sample of a first subframe 815. To provide for continuity of the search space, the line data is mirrored with adjacent lines. The size of the padding depends on the search space size. For example, in some embodiments, the data padding and depadding may be set to half of the search space size. Alternatively, the full size of the search space may be utilized and any redundant lines may be removed after processing. As described previously, this data padding of the frame or matrix of digital ultrasound signal data (RF line data) provides for a greater range of searching and smoother results for subframe matching between and among subframes, such as into the regions adjacent to the image regions near 359° and near 0°, for example.

Referring again to FIGS. 23A-23C, in optional step 540, positional (or resolutional) weighting is applied to the first reference RF line data frame 805 and the second or next RF line data frame 810. The resolution of any ultrasound system tends to degrade by depth due to signal absorption. This is relevant to the IVUS imaging catheter 200, which has a comparatively small diameter (e.g., 5-8 F) and a comparatively limited number of piezoelectric transducer elements 250. Since the noise interference increases as the emitted signal is absorbed, the flow motion characteristics, as well as speckle characteristics, may also change by depth. Additionally, as the IVUS catheter 200 is inside of the vein, the most clinically important blood flow occurs mostly in the near-field (less than 2 cm (20 mm)), including blood flow present in the veins, collaterals, and arteries. After two cm (20 mm), the likelihood of encountering relevant tissue or flow information decreases significantly. As an option in step 540, a resolution weighting is applied to grade the importance of regions, which not only divides the imaging region into multiple sections (described below) but also utilizes linearly decreasing coefficients to reduce the noise effect.

In step 545, the user may determine whether the vessel contour trace 405 will be utilized to constrain the search region of the second or next RF line data frame 810, to confine the speckle searching to the area bounded by the vessel contour trace 405 (e.g., the venous wall 435). Alternatively, when the vessel contour trace 405, the method 500 may constrain the search region of the second or next RF line data frame 810, to confine the speckle searching to the area bounded by the venous wall 435. When the vessel contour trace 405 will be utilized to constrain the search region of the second or next RF line data frame 810 in step 545, the method limits the search region of the second or next RF line data frame 810 to the region bounded by the venous wall 435, step 550. When the vessel contour trace 405 is unavailable or will not be utilized to constrain the search region of the second or next RF line data frame 810 in step 545, the method 500 then proceeds to step 555. Following step 550, the method also proceeds to step 555, but utilizes the constrained search region of the second or next RF line data frame 810.

Following steps 545 or 550, as an option, the first and second RF line data frames 805, 810 are separated into a plurality of separate search regions, step 555, such as for using different search parameters, such as a near-field (with a higher density of data), a mid-field, and a far-field (which may be noisier and have a weaker signal), for example. Different search parameters may be utilized in the different regions, such as the comparative sizes of the first and second subframes 815, 820, thresholds for matching, etc. In addition, if B-mode data is utilized, any time gain compensation which may have been applied may now be suppressed.

Since the signal to noise ratio as well as speckle patterns show different characteristics in these regions, such as near-, mid- and far-fields, the size for the first and second subframes 815, 820 may be varied depending on the depth. For example, in the near field, a comparatively smaller first subframe 815 (as the region of interest (ROI)) is searched in the second or next RF line data frame 810, and size of the first subframe 815 may increase with the depth (far-field). Alternatively, the first and second subframes 815, 820 could be constant along the depth, decrease or increase with depth in a linear or non-linear fashion, or adaptively adjusted based on the image content. For example, if the average motion in a search region for multiple consecutive frames is comparatively small, the size of the search space may be reduced. In addition, using the divided search regions may also improve the parallel processing speed, such as by using local GPU memory for near-field determinations and global memory registers for mid- and far-field determinations.

A first subframe 815 (of a plurality of first subframes 815 of the first reference RF line data frame 805) and a corresponding, larger second subframe 820 (of a plurality of second subframes 820 of the second or next RF line data frame 810) are then selected for search and comparison, referred to as “block matching”, step 560. In some embodiments, each pixel of the first reference RF line data frame 805 may be selected to be the center of a first subframe 815, forming a plurality of first subframes 815 covering the entire first reference RF line data frame 805, so that every pixel of the first reference RF line data frame 805 has a corresponding first subframe 815. Other methods of selecting first subframes 815 are considered equivalent and within the scope of the disclosure, such as dividing the first reference RF line data frame 805 into a plurality of adjacent first subframes 815 covering the entire first reference RF line data frame 805 (e.g., similar to all of the adjacent squares of a checkerboard). Block matching is then performed for each first subframe 815 of the first reference RF line data frame 805 with each corresponding portion of the second subframe 820 of the second or next RF line data frame 810, step 565, generating a similarity score for that selected comparison. As indicated previously, the interior of a vein appears black in an ultrasound image, with a significant portion of the pixels in the ultrasound image effectively having a zero (or close to zero) value, and with the speckle appearing as pixels having non-zero values, such as illustrated with pixels 880 in the first subframe 815 illustrated in FIG. 28, for example. The closest or best matching section 875 of the second subframe 820 is selected and displacement calculations (displacements 855, 860) are made (step 580) to detect motion. It should be noted that the matching section 875 has a similar, but not exact, arrangement of non-zero pixels 885, indicating speckle movement (blood flow) between the successive first reference RF line data frame 805 and the second or next RF line data frame 810.

When there are additional portions of the second subframe 820 for searching, step 570, the block matching continues, returning to step 565 and iterating. When the block search of the first subframe 815 with the second subframe 820 of the second or next RF line data frame 810 is completed in step 570, such that there are no remaining portions of the second subframe 820 for searching, the method determines whether there are additional first and/or second subframes 815, 820 for searching, step 575, and if so, this block matching continues, returning to step 560 and iterating, selecting the next pair of a first subframe 815 and a corresponding, larger second subframe 820 for search and comparison. When there are no additional first subframes 815 and/or second subframes 820 for searching in step 575, the block matching has been completed for the first reference RF line data frame 805 and the second or next RF line data frame 810, and the method proceeds to various post-processing steps.

In some embodiments, as described above, the second subframe 820 for search and comparison is comparatively larger than the first subframe 815. As illustrated in FIG. 26, a first subframe 815 is compared to different regions of a corresponding second subframe 820. As illustrated in FIG. 27A, a first subframe 815 is compared to different regions of a corresponding second subframe 820, using a “sliding window” approach. In FIG. 27A, the first subframe 815 is compared to a first region 845A of the corresponding second subframe 820. In FIG. 27B, the first subframe 815 is compared to a second region 845B of the corresponding second subframe 820, with the second region 845B displaced in a first dimension from the first region 845A by a predetermined distance 830, as the sliding window for comparison, such as one pixel. As this comparison process continues, in FIG. 27C, the first subframe 815 is compared to a third region 845C of the corresponding second subframe 820, with the third region 845C displaced in first and second dimensions from the first region 845A by predetermined distances 835, 840. Finally, the comparison of the first subframe 815 with the second subframe 820 is complete, as illustrated in FIG. 27D, with the first subframe 815 compared to a last or final region 845D of the corresponding second subframe 820.

In some embodiments, block matching may be used to implement speckle tracking using different distance or other similarity metrics, to detect the most similar speckle pattern for each pair of first and second subframes 815, 820 of each successive first reference RF line data frame 805 and second, search and comparison RF line data frame 810. The output of the block matching generally comprises one or more matrices which are the Euclidean distance values, similarity measurements, and/or displacements on x and y dimensions, for example. Displacement can be absolute or normalized in respect to kernel size. For instance, in FIG. 28, region or portion 875 of the second subframe 820 has been found to be the closest match, and a displacement may be determined or measured using the center 850 of the first subframe 815 and the center 850A of the matching section 875, for example, with the center 850A of the matching section 875 having been displaced in the x dimension by displacement 855 and in the y-dimension by a displacement 860, and a Euclidean distance may be determined (e.g., as the square root of the sum of the squares of each displacement amount).

A wide variety of similarity metrics may be utilized to determine the best match (if any) of the speckle pattern within a first subframe 815 to a selected portion of the corresponding second subframe 820, as each non-zero pixel (representing speckle) of the first subframe 815 is compared to the non-zero pixels of a selected portion of the corresponding second subframe 820. For example, any of a plurality of similarity metrics and combinations of various similarity metrics may be utilized for block matching, including cross-correlation, normalized cross-correlation, sum of squared differences (SSD), normalized sum of squared differences, and/or correlation coefficient-based metrics. In finding a block match, additionally, a degree of matching may also be implemented, such as only finding a block match above a predetermined threshold, also for example. In some embodiments, the best matching section 875 may be determined as a peak similarity value or global maximum value compared to a mean similarity value (e.g., as a ratio of global maximum value or peak similarity value to a mean similarity value), for example. In another representative embodiment, the best matching section 875 may be determined with additional thresholding, as a ratio of global maximum value or peak similarity value to a mean similarity value, which ratio is greater than a predetermined threshold, also for example. During the search process of comparing the selected first subframe 815 with the various second subframes 820, a plurality of local maximum similarity values may be determined, with the final global similarity maximum selected as the highest value from among the plurality of local maximum similarity values. It should also be emphasized that in some embodiments, all of the searching and block matching has been performed using RF line data, which has not yet been converted to Cartesian space for image display.

Following step 575, post-processing begins. As indicated above, such post-processing may include various types of thresholding or otherwise filtering out unreliable information, for example. In step 580, as an option, all of the sectioned data from all of the block matching (such as performed in various GPU or other processing cores 148) is converted into a single RF line data frame which has the same input size as the first reference RF line data frame 805 and the second or next RF line data frame 810. Such merging may also incorporate any type of blending methods, for instance alpha blending, gaussian pyramid blending, or simple region compounding, for example.

In step 585, spatial and temporal smoothing is applied, such as to multiple frames, to eliminate noise and sustain temporal consistency, for example. Given that soft tissues like blood vessels and organs have smoother edges instead of sharp transitions, a low pass filter may be applied to obtain a more fluid-like flow motion mask, smoothing across multiple frames in the vicinity of each matching section 875. In some embodiments, a 5×5 pixel block is utilized (as an example), and the average or mean of the pixel values is taken, which is also known as Gaussian blurring. In another representative embodiment, a fluid-like regularization is applied to displacement field, which can be for instance approximated by Gaussian filtering (e.g., performing a convolution operation on the auxiliary field with the Gaussian kernel with a scale σ). Alternatively, a smooth thresholding is applied to the ratio of non-zero pixels to number of pixels for a selection of a subvolume and the mean of all non-zero values are referred as displacement for the selected subvolume. In addition, any outliers may be eliminated or dropped, as an option, such as pixels which appear in one frame and disappear in the next frame. A wide variety of smoothing techniques may be utilized equivalently, such as mean, median, Gaussian, majority voting, mean scoring, weighting, and local averages, for example.

In step 590, additional filtering is performed using a connected components analysis (also referred to as “blob” filtering). Connected component analysis may be used to identify and analyze connected regions (also known as “blobs”) within an image, such as using near template matching. It recognizes the fact that both blood flow and tissue are always depicted using more than a single pixel, and if the pixels in the vicinities do not show the same behavior, it may be considered to be noise. In some embodiments, a color mask value (representing blood flow) may be eliminated if it is below a predetermined threshold. Alternative mask post-processing methods may also be used, including simple thresholding, watershed algorithms, various morphological operations (erosion, dilatation, opening, closing), and deep learning-based methods, for example.

As indicated above, one of the outputs of the block matching is a matrix of similarity values indicative of blood flow (comprising the highest similarity values for all of the block matching of each of the first subframes 815 of the first reference RF line data frame 805 with each of the corresponding second subframes 820 of the second or next RF line data frame 810). In step 595, this matrix of similarity values based upon RF line data is converted is to Cartesian coordinates, to provide a color image or mask to display as a visual image of the blood flow. As part of this conversion, additional smoothing or blurring may also be performed. For example, a weighted smoothing operation where the weights are configured based on the spatial information of a subframe after Cartesian conversion is used.

In step 605, blood flow metrics are determined, such as displacement. In the block matching steps, there are two output matrices: a first similarity matrix, described above and used to convert the RF line data to Cartesian coordinates to generate the visual image, and a second displacement metrics matrix, indicating the amount of speckle movement indicative of blood flow. The first similarity matrix is used in various thresholding and filter steps to select only sufficiently similar matchings. The second displacement metrics matrix indicates the magnitude and direction of movement of the first subframes 815. In step 605, the magnitude of the displacement is determined, such as by taking the square root of the sum of squared difference between x and y values. If the displacement magnitude becomes higher, for example, the pixel may become redder to demonstrate the faster motion. (It should be noted that the x and y values would take negative values if the best matching block is detected towards left and/or down but that information disappears after calculating the squares.) In some embodiments, a distance between two consecutive RF line data frames, is known, such as based upon pullback of the IVUS catheter 200 within the vein during imaging. When the IVUS catheter 200 is pulled back at a constant rate, a time difference between two consecutive frames is also known. This time information, as a third dimension, may be appended to the spatial (x,y) direction in order to predict 3D motion of one or more blood cells. In some embodiments, a distance between two such consecutive RF line data frames is calculated using hardware-free image processing methods such as optical flow, deep learning dense flow, or hom0graphy, for example.

In step 605, the user-selected blood flow imaging parameters are applied. As indicated above, in start step 505, the user may select blood flow detection as an option, and additionally select various color parameters for the blood flow detection, such as using the GUI 900 illustrated in FIG. 29, such as color sensitivity 905, color opacity 910, and color depth 915, in addition to other B-mode parameters.

Color sensitivity 905 is a parameter for changing the sensitivity for the various thresholds used in determining similarity metrics and displacement. As previously described, the speckle tracking provides a matching score which increases as the first subframe 815 and second subframe 820 are more similar, and a displacement matrix which represents how much the first subframe 815 has moved. The sensitivity setting is to adapt the matching based on the user preference, to tune accepted matching score values as well as set the limits for displacement, which would allow slower or faster displacement measures based on the parameter settings.

Color opacity 910 is a parameter which allows a user to determine the blending ratio between the B-mode image and the color image (or mask) representing blood flow. After the color image or mask is produced, it is displayed superimposed on top of or over the B-mode image, and as the color opacity 910 increases, the color image or mask overwrites the B-mode image more. In other words, if the opacity is 0, the transparency is 100% and the user would only see the B-mode image. Conversely, when the color opacity is 1 (e.g., the highest setting), the B-mode image would be completely superseded.

Color depth 915 is a parameter which allows the user to determine the anatomical region of interest for the generation of the color image. In some embodiments, for example, an IVUS catheter 200 scan may penetrated to as much as four cm k (40 mm), and the color blood flow image may extend to the entire scanning area or may be more limited, such as to limit the blood flow imaging to the near field. Limiting the color depth 915 to the near field, for example, may be advantageous, allowing a higher frame rate and lower amount of computation, for example.

Lastly, in step 610, the color image or mask is merged, blended with or superimposed on the B-mode IVUS image, as illustrated in FIG. 21, and displayed to the user, such as displayed on the image output display 155. The method 500 may also provide various types of blending, such as alpha-blending, and utilized different colors to indicate the speed of blood flow, as mentioned above. Not separately illustrated, additional information may also be displayed, such as by using colored arrows to indicate the direction and magnitude of blood flow. Following display of the merged blood flow color and B-mode image, the method may end, return step 615. While not separately illustrated in FIGS. 23A-23C, it should be noted that the method 500 may be repeated for successive RF line data frames received from the IVUS catheter 200 imaging.

It should also be noted that the method 500 may be performed in real-time, during scan acquisition, or at a later time, post-acquisition. In the various embodiments, a dual signal path (including flow imaging) may be enabled and the additional blood flow data is saved along the primary signal path. Saving additional data allows the computation of the blood flow image in the review mode (post-acquisition), thereby significantly reducing procedure time. This can also be done using a single signal path (B-mode). In addition, additional frames may be saved prior to any display of an image to the user, in order to provide a temporal smoothing to the blood flow image, for example.

### V. Examples

The following examples are illustrative of several embodiments of the present technology:
1. A computing system implemented method of generating a vessel contour trace from an intravascular ultrasound (“IVUS”) image, the computing system including an IVUS console having a processor circuit and an image output display, the method comprising:
using the IVUS console, receiving a first frame of RF line data for the IVUS image;
using the processor circuit of the IVUS console, converting the first frame of RF line data to Cartesian coordinates and displaying the IVUS image;
using the IVUS console, receiving a user selection for the generation of the vessel contour trace;
using the processor circuit of the IVUS console, generating a segmentation of the IVUS image to identify the vessel of interest for the vessel contour trace;
using the processor circuit of the IVUS console, generating an initial vessel contour trace from the generated segmentation;
using the processor circuit of the IVUS console, generating an interim vessel contour trace using an active contour model initialized with the initial vessel contour trace and constrained with a statistical vein shape model (“SSM”); and
using the processor circuit of the IVUS console, when the interim vessel contour trace meets or exceeds accuracy criteria, displaying on the image output display a complete vessel contour trace comprising the interim vessel contour trace with user-interactable key nodes or control points.
2. The method of example 1, further comprising:
using the processor circuit of the IVUS console, generating and displaying on the image output display one or more selected biometrics.
3. The method of either example 1 or example 2, further comprising:
using the IVUS console, removing any ringdown artifact from the ultrasound image.
4. The method of any of examples 1-3, wherein the step of receiving a user selection for the generation of the vessel contour trace further comprises:
using the processor circuit of the IVUS console, when a user has selected autocorrection, using a user-provided manual trace as the initial vessel contour trace and displaying the user-provided manual trace on the image output display.
5. The method of any of examples 1-4, wherein the step of receiving a user selection for the generation of the vessel contour trace further comprises:
using the processor circuit of the IVUS console, when a user has selected autocompletion, using user-provided key nodes as accurate or true key nodes in the complete vessel contour trace.
6. The method of any of examples 1-5, further comprising:
using the processor circuit of the IVUS console, converting the initial vessel contour trace, from RF line data, into a Cartesian space coordinate system.
7. The method of any of examples 1-6, further comprising:
using the processor circuit of the IVUS console, modifying the vessel contour trace in the region between an IVUS catheter and a vessel wall.
8. The method of any of examples 1-7, further comprising:
using the processor circuit of the IVUS console, when the interim vessel contour trace does not meet or exceed the accuracy criteria, generating a prompt to a user to select or input one or more key nodes or other control points.
9. The method of any of examples 1-8, further comprising:
using the processor circuit of the IVUS console, when a user modifies the generated vessel contour trace, generating a next vessel contour trace incorporating the user modifications.
10. The method of any of examples 1-9, wherein the step of generating the segmentation further comprises:
using the processor circuit of the IVUS console, generating a plurality of RF line data channels;
using the processor circuit of the IVUS console, combining the plurality of RF line data channels to generate a combined RF line data channel;
using the processor circuit of the IVUS console, generating a second frame of RF line data having an orientation shift from the first frame of RF line data;
using the processor circuit of the IVUS console, and using the first frame of RF line data, obtaining a first segmentation prediction result;
using the processor circuit of the IVUS console, and using the second frame of RF line data, obtaining a second segmentation prediction result;
using the processor circuit of the IVUS console, rotating the second segmentation prediction result to remove the orientation shift;
using the processor circuit of the IVUS console, combining or merging the first segmentation prediction result with the orientation-shifted second segmentation prediction result to generate a combined segmentation prediction result; and
using the processor circuit of the IVUS console, extracting edge data from the combined segmentation prediction result to generate the initial vessel contour trace.
11. The method of example 10, wherein the step of generating the plurality of RF line data channels further comprises:
using the processor circuit of the IVUS console, creating a first, positionally weighted RF line data channel;
using the processor circuit of the IVUS console, creating a second, depth normalization RF line data channel; and
using the processor circuit of the IVUS console, using the first frame of RF line data as a third RF line data channel.
12. The method of example 10, further comprising:
using the processor circuit of the IVUS console, providing data padding to the combined RF line data channel.
13. The method of example 12, further comprising:
using the processor circuit of the IVUS console, de-padding the combined segmentation prediction result and re-sizing the IVUS image.
14. The method of example 10, further comprising:
using the processor circuit of the IVUS console, filtering the combined segmentation prediction result to retain the largest or maximum segmentation indicative of the vessel of interest.
15. The method of any of examples 1-14, wherein the step of generating the interim vessel contour trace further comprises:
using the processor circuit of the IVUS console, iteratively moving the initial vessel contour trace toward the vessel wall edge by optimizing an energy function.
16. The method of example 15, wherein the energy function comprises a plurality of parameters or constraints, the plurality of parameters or constraints comprising a statistical shape energy or constraint; an edge-based energy or constraint; and a region-based energy or constraint.
17. The method of example 16, wherein the edge-based energy constraint utilizes the intensity gradient in the image to move the initial vessel contour trace toward the highest gradient indicative of the edge of the vessel wall.
18. The method of example 16, wherein the region-based energy constraint maximizes the contrast between the intensity of the data averaged within the initial vessel contour trace, and the intensity of the data averaged over an elliptical shell.
19. The method of any of examples 1-18, wherein the step of generating the interim vessel contour trace further comprises:
using the processor circuit of the IVUS console, fitting a variation of an ellipse to the initial vessel contour trace using the SSM.
20. The method of any of examples 1-19, further comprising:
using the processor circuit of the IVUS console, modify the interim vessel contour trace to account for a contour of an edge of the vessel wall in the vicinity of a ringdown artifact.
21. The method of any of examples 1-20, further comprising:
using the using the processor circuit of the IVUS console, training the SSM model by:
sampling a fixed number of points from each shape or contour of a training data set and geometrically align and scaling the training data;
computing a mean shape or contour “x̅”; and
extracting the main modes of shape or contour variation by calculating the eigenvalues of the covariance matrix (Cov=M·M^{T}) of all of the training data to generate a matrix of eigenvalues (λ₁, λ₂, λ₃, λ₄, . . . λ_{N}), a corresponding matrix of variations (e₁, e₂, e₃, e₄, . . . e_{N}, with each “e” being a vector), and corresponding weightings (w₁, w₂, w₃, w₄, . . . w_{N}).
22. The method of example 21, wherein the SSM model may be represented as a mean with weighted (“wᵢ”) variations:

$$x = \overline{x} + {\sum }_{i = 0}^{n} {w}_{i} {e}_{i} .$$

23. The method of example 21, further comprising:
using the using the processor circuit of the IVUS console, fitting the initial or interim vessel contour trace to the SSM model by:
sampling and scaling points of the initial or interim vessel contour trace to match the coordinate space of the mean shape;
aligning the scaled initial or interim vessel contour trace with the mean shape (x̅);
applying the SSM model to the aligned shape (“x”) to obtain the weightings: wᵢ=eᵢ(x−x̅)^{T}.
24. The method of example 23, further comprising:
using the using the processor circuit of the IVUS console, determining that the interim vessel contour trace meets or exceeds accuracy criteria when the weightings wi for the initial or interim vessel contour trace are within a predetermined amount or level of the corresponding eigenvalues (λ₁, λ₂, λ₃, λ₄, . . . ).
25. The method of example 24, further comprising:
using the using the processor circuit of the IVUS console, determining that the interim vessel contour trace meets or exceeds accuracy criteria when: |wᵢ|≤\sqrt{3}|λi|.
26. An intravascular ultrasound (“IVUS”) console for generating a vessel contour trace from an IVUS image, the IVUS console comprising:
a communication interface to receive a first frame of RF line data for the IVUS image and receive a user selection for the generation of the vessel contour trace;
a memory circuit configured to store training data;
an image output display; and
a processor circuit coupled to the communication interface, to the memory circuit and to the image output display, the processor circuit configured to: convert the first frame of RF line data to Cartesian coordinates and display the IVUS image on the image output display; generate a segmentation of the IVUS image to identify the vessel of interest for the vessel contour trace; generate an initial vessel contour trace from the generated segmentation; generate an interim vessel contour trace using an active contour model initialized with the initial vessel contour trace and constrained with a statistical vein shape model (“SSM”); and the processor circuit configured, when the interim vessel contour trace meets or exceeds accuracy criteria, to display on the image output display a complete vessel contour trace comprising the interim vessel contour trace with user-interactable key nodes or control points.
27. The IVUS console of example 26, wherein the processor circuit is further configured to generate and displaying on the image output display one or more selected biometrics.
28. The IVUS console of either example 26 or example 27, wherein the processor circuit is further configured to remove any ringdown artifact from the ultrasound image.
29. The IVUS console of any of examples 26-28, wherein the processor circuit is further configured, when a user has selected autocorrection, to use a user-provided manual trace as the initial vessel contour trace and to display the user-provided manual trace on the image output display.
30. The IVUS console of any of examples 26-29, wherein the processor circuit is further configured, when a user has selected autocompletion, to use user-provided key nodes as accurate or true key nodes in the complete vessel contour trace.
31. The IVUS console of any of examples 26-30, wherein the processor circuit is further configured to convert the initial vessel contour trace, from RF line data, into a Cartesian space coordinate system.
32. The IVUS console of any of examples 26-31, wherein the processor circuit is further configured to modify the vessel contour trace in the region between an IVUS catheter and a vessel wall.
33. The IVUS console of any of examples 26-32, wherein the processor circuit is further configured, when the interim vessel contour trace does not meet or exceed the accuracy criteria, to generate a prompt to a user to select or input one or more key nodes or other control points.
34. The IVUS console of any of examples 26-33, wherein the processor circuit is further configured, when a user modifies the generated vessel contour trace, to generate a next vessel contour trace incorporating the user modifications.
35. The IVUS console of any of examples 26-34, wherein the processor circuit is further configured to generate the segmentation by: generating a plurality of RF line data channels; combining the plurality of RF line data channels to generate a combined RF line data channel; generating a second frame of RF line data having an orientation shift from the first frame of RF line data; obtaining a first segmentation prediction result using the first frame of RF line data; obtaining a second segmentation prediction result using the second frame of RF line data; rotating the second segmentation prediction result to remove the orientation shift; combining or merging the first segmentation prediction result with the orientation-shifted second segmentation prediction result to generate a combined segmentation prediction result; and extracting edge data from the combined segmentation prediction result to generate the initial vessel contour trace.
36. The IVUS console of example 35, wherein the processor circuit is further configured to generate the plurality of RF line data channels by: creating a first, positionally weighted RF line data channel; creating a second, depth normalization RF line data channel; and using the first frame of RF line data as a third RF line data channel.
37. The IVUS console of example 36, wherein the processor circuit is further configured to provide data padding to the combined RF line data channel.
38. The IVUS console of any of examples 26-37, wherein the processor circuit is further configured to de-pad the combined segmentation prediction result and re-size the IVUS image.
39. The IVUS console of example 35, wherein the processor circuit is further configured to filter the combined segmentation prediction result to retain the largest or maximum segmentation indicative of the vessel of interest.
40. The IVUS console of any of examples 26-39, wherein the processor circuit is further configured to generate the interim vessel contour trace by iteratively moving the initial vessel contour trace toward the vessel wall edge by optimizing an energy function.
41. The IVUS console of example 40, wherein the energy function comprises a plurality of parameters or constraints, the plurality of parameters or constraints comprising a statistical shape energy or constraint; an edge-based energy or constraint; and a region-based energy or constraint.
42. The IVUS console of example 41, wherein the edge-based energy constraint utilizes the intensity gradient in the image to move the initial vessel contour trace toward the highest gradient indicative of the edge of the vessel wall.
43. The IVUS console of example 41, wherein the region-based energy constraint maximizes the contrast between the intensity of the data averaged within the initial vessel contour trace, and the intensity of the data averaged over an elliptical shell.
44. The IVUS console of any of examples 26-43, wherein the processor circuit is further configured to generate the interim vessel contour trace by fitting a variation of an ellipse to the initial vessel contour trace using the SSM.
45. The IVUS console of any of examples 26-44, wherein the processor circuit is further configured to modify the interim vessel contour trace to account for a contour of an edge of the vessel wall in the vicinity of a ringdown artifact.
46. The IVUS console of any of examples 26-45, wherein the processor circuit is further configured to train the SSM model by:
sampling a fixed number of points from each shape or contour of a training data set and geometrically align and scaling the training data;
computing a mean shape or contour “x̅”; and
extracting the main modes of shape or contour variation by calculating the eigenvalues of the covariance matrix (Cov=M·M^{T}) of all of the training data to generate a matrix of eigenvalues (λ₁, λ₂, λ₃, λ₄, . . . λ_{N}), a corresponding matrix of variations (e₁, e₂, e₃, e₄, . . . e_{N}, with each “e” being a vector), and corresponding weightings (w₁, w₂, w₃, w₄, . . . w_{N}).
47. The IVUS console of example 46, wherein the SSM model may be represented as a mean with weighted (“wᵢ”) variations:

$$x = \overline{x} + {\sum }_{i = 0}^{n} {w}_{i} {e}_{i} .$$

48. The IVUS console of example 46, wherein the processor circuit is further configured to fit the initial or interim vessel contour trace to the SSM model by:
sampling and scaling points of the initial or interim vessel contour trace to match the coordinate space of the mean shape;
aligning the scaled initial or interim vessel contour trace with the mean shape (x̅);
applying the SSM model to the aligned shape (“x”) to obtain the weightings: wᵢ=eᵢ(x−x̅)^{T}.
49. The IVUS console of example 48, wherein the processor circuit is further configured to determine that the interim vessel contour trace meets or exceeds accuracy criteria when the weightings wi for the initial or interim vessel contour trace are within a predetermined amount or level of the corresponding eigenvalues (λ₁, λ₂, λ₃, λ₄, . . . ).
50. The IVUS console of example 49, wherein the processor circuit is further configured to determine that the interim vessel contour trace meets or exceeds accuracy criteria when: |wᵢ|≤\sqrt{3}|λi|.
51. A computing system implemented method of detecting blood flow from intravascular ultrasound (“IVUS”) and generating a blood flow IVUS image, the computing system including an IVUS console having a processor circuit and an image output display, the method comprising:
using the IVUS console, receiving a plurality of frames of RF line data for the blood flow IVUS image, the plurality of frames of RF line data comprising a first frame of RF line data and a second frame of RF line data;
using the processor circuit of the IVUS console, selecting a first subframe of a plurality of first subframes of the first RF line data frame;
using the processor circuit of the IVUS console, selecting a second subframe of a plurality of second subframes of the second RF line data frame for search and comparison, the second subframe larger than the first subframe;
using the processor circuit of the IVUS console, performing block matching for each selected first subframe, of the plurality of first subframes, with each corresponding portion of the selected second subframe, of the plurality of second subframes, and generating a similarity score for each selected comparison;
using the processor circuit of the IVUS console, and using of a plurality of similarity scores from the block matching, generating a third RF line data frame as a matrix of the plurality of similarity scores;
using the processor circuit of the IVUS console, converting the matrix of the plurality of similarity scores to Cartesian coordinates to provide a color image or mask to display as a visual image of the blood flow;
using the processor circuit of the IVUS console, generating a brightness-mode (“B-mode”) IVUS image from one or more frames of RF line data of the plurality of frames of RF line data;
using the processor circuit of the IVUS console, merging or superimposing the color image or mask on or with the B-mode IVUS image to generate the blood flow IVUS image; and
using the processor circuit of the IVUS console, displaying the blood flow IVUS image on the image output display.
52. The method of example 51, wherein the block matching comprises a comparison of at least one first pattern of a plurality of speckle pixels within the selected first subframe with at least one second pattern of a plurality of speckle pixels within the selected second subframe.
53. The method of either example 51 or example 52, further comprising:
using the IVUS console, receiving a user selection of or determining one or more signal acquisition modes.
54. The method of any of examples 51-53, further comprising:
using the IVUS console, receiving a user selection of blood flow detection and color parameters.
55. The method of any of examples 51-54, further comprising:
using the processor circuit of the IVUS console, applying the user-selected blood flow detection and color parameters.
56. The method of any of examples 51-55, further comprising:
using the processor circuit of the IVUS console, applying one or more thresholds to RF line data of the first or second frames of RF line data.
57. The method of any of examples 51-56, further comprising:
using the processor circuit of the IVUS console, removing any ringdown artifact from the first and second RF line data frames.
58. The method of any of examples 51-57, further comprising:
using the processor circuit of the IVUS console, performing clutter filtering of the first and second RF line data frames.
59. The method of any of examples 51-58, further comprising:
using the processor circuit of the IVUS console, providing data padding to the second RF line data frame.
60. The method of any of examples 51-59, further comprising:
using the processor circuit of the IVUS console, applying positional (or resolutional) weighting to the first and second RF line data frames.
61. The method of any of examples 51-60, further comprising:
using the processor circuit of the IVUS console, limiting a search region of the second RF line data frame to a region bounded by a vessel contour trace.
62. The method of any of examples 51-61, further comprising:
using the processor circuit of the IVUS console, separating the first and second RF line data frames into a plurality of separate search regions.
63. The method of any of examples 51-62, further comprising:
using the processor circuit of the IVUS console, applying spatial and temporal smoothing to the third RF line data frame.
64. The method of any of examples 51-63, further comprising:
using the processor circuit of the IVUS console, filtering the third RF line data frame using a connected components analysis.
65. The method of any of examples 51-64, further comprising:
using the processor circuit of the IVUS console determining one or more blood flow metrics, the one or more blood flow metrics comprising a displacement determination.
66. The method of any of examples 51-65, further comprising:
using the processor circuit of the IVUS console, generating and displaying on the image output display one or more selected biometrics.
67. An intravascular ultrasound (“IVUS”) console for detecting blood flow and generating a blood flow IVUS image, the IVUS console comprising:
a communication interface configured to receive a plurality of frames of RF line data for the blood flow IVUS image, the plurality of frames of RF line data comprising a first frame of RF line data and a second frame of RF line data;
a memory circuit;
an image output display; and
a processor circuit coupled to the communication interface, to the memory circuit and to the image output display, the processor circuit configured to: select a first subframe of a plurality of first subframes of the first RF line data frame; select a second subframe of a plurality of second subframes of the second RF line data frame for search and comparison, the second subframe larger than the first subframe; perform block matching for each selected first subframe, of the plurality of first subframes, with each corresponding portion of the selected second subframe, of the plurality of second subframes, and generate a similarity score for each selected comparison; use a plurality of similarity scores from the block matching and generate a third RF line data frame as a matrix of the plurality of similarity scores; convert the matrix of the plurality of similarity scores to Cartesian coordinates to provide a color image or mask to display as a visual image of the blood flow; generate a brightness-mode (“B-mode”) IVUS image from one or more frames of RF line data of the plurality of frames of RF line data; and merge or superimpose the color image or mask on or with the B-mode IVUS image to generate the blood flow IVUS image to display on the image output display.
68. The IVUS console of example 67, wherein the block matching comprises a comparison of at least one first pattern of a plurality of speckle pixels within the selected first subframe with at least one second pattern of a plurality of speckle pixels within the selected second subframe.
69. The IVUS console of either example 67 or example 68, wherein the communication interface is further configured to receive a user selection of one or more signal acquisition modes.
70. The IVUS console of any of examples 67-69, wherein the processor circuit is further configured to determine one or more signal acquisition modes.
71. The IVUS console of any of examples 67-70, wherein the communication interface is further configured to receive a user selection of blood flow detection and color parameters.
72. The IVUS console of example 71, wherein the processor circuit is further configured to apply the user-selected blood flow detection and color parameters.
73. The IVUS console of any of examples 67-72, wherein the processor circuit is further configured to apply one or more thresholds to RF line data of the first or second frames of RF line data.
74. The IVUS console of any of examples 67-73, wherein the processor circuit is further configured to remove any ringdown artifact from the first and second RF line data frames.
75. The IVUS console of any of examples 67-74, wherein the processor circuit is further configured to perform clutter filtering of the first and second RF line data frames.
76. The IVUS console of any of examples 67-75, wherein the processor circuit is further configured to provide data padding to the second RF line data frame.
77. The IVUS console of any of examples 67-76, wherein the processor circuit is further configured to apply positional (or resolutional) weighting to the first and second RF line data frames.
78. The IVUS console of any of examples 67-77, wherein the processor circuit is further configured to limit a search region of the second RF line data frame to a region bounded by a vessel contour trace.
79. The IVUS console of any of examples 67-78, wherein the processor circuit is further configured to separate the first and second RF line data frames into a plurality of separate search regions.
80. The IVUS console of any of examples 67-79, wherein the processor circuit is further configured to apply spatial and temporal smoothing to the third RF line data frame.
81. The IVUS console of any of examples 67-80, wherein the processor circuit is further configured to filter the third RF line data frame using a connected components analysis.
82. The IVUS console of any of examples 67-81, wherein the processor circuit is further configured to determine one or more blood flow metrics, the one or more blood flow metrics comprising a displacement determination.
83. The IVUS console of any of examples 67-82, wherein the processor circuit is further configured to generate one or more selected biometrics for display on the image output display.
84. A method of detecting blood flow and generating a vessel contour trace from an intravascular ultrasound (“IVUS”) image of a vessel, the method comprising:
receiving a plurality of frames of radiofrequency (RF) line data for the IVUS image;
generating an interim vessel contour trace of the vessel based on one or more of the frames of RF line data;
generating one or more user-interactable key nodes along the interim vessel contour trace;
generating a color mask representative of blood flow through the vessel based on one or more of the frames of RF line data; and
displaying a composite image on an output display, wherein the composite image comprises (a) the IVUS image, (b) the interim vessel trace and the user-interactable key nodes overlaid over the IVUS image, and (c) the color mask representative of blood flow through the vessel overlaid over the IVUS image.
85. The method of example 84, further comprising:
modifying the interim vessel contour trace in response to user interaction with the one or more user-interactable key nodes.
86. The method of either example 84 or example 85, wherein generating the interim vessel contour trace comprises:
generating a segmentation of the IVUS image to identify the vessel;
generating an initial vessel contour trace from the segmentation; and
refining the initial vessel contour trace using an active contour model constrained with a statistical vein shape model.
87. The method of any of examples 84-86, wherein generating the color mask representative of blood flow comprises:
selecting a first subframe from a first frame of the plurality of frames of RF line data;
selecting a second subframe from a second frame of the plurality of frames of RF line data;
performing block matching between the first subframe and the second subframe to generate similarity scores; and
converting the similarity scores to Cartesian coordinates to provide the color mask.
88. The method of any of examples 84-87, further comprising:
removing a ringdown artifact from one or more of the plurality of frames of RF line data prior to generating the interim vessel contour trace or the color mask.
89. The method of any of examples 84-88, further comprising:
applying one or more thresholds to the plurality of frames of RF line data; and
performing clutter filtering on the plurality of frames of RF line data based on the one or more thresholds.
90. The method of any of examples 84-89, further comprising:
generating and displaying on the output display one or more selected biometrics.
91. A method of generating a vessel contour trace from an intravascular ultrasound (“IVUS”) image, the method comprising:
receiving a first frame of RF line data for the IVUS image;
converting the first frame of RF line data to Cartesian coordinates and displaying the IVUS image;
receiving a user selection for the generation of the vessel contour trace;
generating a segmentation of the IVUS image to identify a vessel of interest for the vessel contour trace;
generating an initial vessel contour trace from the generated segmentation;
generating an interim vessel contour trace using an active contour model initialized with the initial vessel contour trace and constrained with a statistical vein shape model; and
displaying on an image output display, when the interim vessel contour trace meets or exceeds accuracy criteria, a complete vessel contour trace comprising the interim vessel contour trace with user-interactable key nodes or control points.
92. The method of example 91, further comprising:
generating and displaying on the image output display one or more selected biometrics.
93. The method of either example 91 or example 92, further comprising:
removing any ringdown artifact from the IVUS image.
94. The method of any of examples 91-93, wherein receiving a user selection for the generation of the vessel contour trace further comprises:
using a user-provided manual trace as the initial vessel contour trace and displaying the user-provided manual trace on the image output display when a user has selected autocompletion.
95. The method of any of examples 91-94, wherein receiving a user selection for the generation of the vessel contour trace further comprises:
using user-provided key nodes as accurate or true key nodes in the complete vessel contour trace when a user has selected autocompletion.
96. The method of any of examples 91-95, further comprising:
converting the initial vessel contour trace, from RF line data, into a Cartesian space coordinate system.
97. The method of any of examples 91-96, further comprising:
modifying the vessel contour trace in a region between an IVUS catheter and a vessel wall.
98. A method of detecting blood flow from intravascular ultrasound (“IVUS”) and generating a blood flow IVUS image, the method comprising:
receiving a plurality of frames of RF line data for the blood flow IVUS image, the plurality of frames of RF line data comprising a first frame of RF line data and a second frame of RF line data;
selecting a first subframe of a plurality of first subframes of the first RF line data frame;
selecting a second subframe of a plurality of second subframes of the second RF line data frame for search and comparison, the second subframe larger than the first subframe;
performing block matching for each selected first subframe, of the plurality of first subframes, with each corresponding portion of the selected second subframe, of the plurality of second subframes, and generating a similarity score for each selected comparison;
generating a third RF line data frame as a matrix of the plurality of similarity scores using a plurality of similarity scores from the block matching;
converting the matrix of the plurality of similarity scores to Cartesian coordinates to provide a color image or mask to display as a visual image of the blood flow;
generating a brightness-mode (“B-mode”) IVUS image from one or more frames of RF line data of the plurality of frames of RF line data;
merging or superimposing the color image or mask on or with the B-mode IVUS image to generate the blood flow IVUS image; and
displaying the blood flow IVUS image on an image output display.
99. The method of example 98, wherein the block matching comprises a comparison of at least one first pattern of a plurality of speckle pixels within the selected first subframe with at least one second pattern of a plurality of speckle pixels within the selected second subframe.
100. The method of either example 98 or example 99, further comprising:
receiving a user selection of or determining one or more signal acquisition modes.
101. The method of any of examples 98-100, further comprising:
receiving a user selection of blood flow detection and color parameters; and
applying the user selection of blood flow detection and color parameters.
102. The method of any of examples 98-101, further comprising:
performing clutter filtering of the first and second RF line data frames.
103. The method of any of examples 98-102, further comprising:
applying one or more thresholds to RF line data of the first or second frames of RF line data.

### VI. Conclusion

The above detailed descriptions of embodiments of the technology are not intended to be exhaustive or to limit the technology to the precise form disclosed above. Although specific embodiments of, and examples for, the technology are described above for illustrative purposes, various equivalent modifications are possible within the scope of the technology as those skilled in the relevant art will recognize. For example, although steps are presented in a given order, alternative embodiments may perform steps in a different order. The various embodiments described herein may also be combined to provide further embodiments.

From the foregoing, it will be appreciated that specific embodiments of the technology have been described herein for purposes of illustration, but well-known structures and functions have not been shown or described in detail to avoid unnecessarily obscuring the description of the embodiments of the technology. Where the context permits, singular or plural terms may also include the plural or singular term, respectively.

Moreover, unless the word “or” is expressly limited to mean only a single item exclusive from the other items in reference to a list of two or more items, then the use of “or” in such a list is to be interpreted as including (a) any single item in the list, (b) all of the items in the list, or (c) any combination of the items in the list. Additionally, the term “comprising” is used throughout to mean including at least the recited feature(s) such that any greater number of the same feature and/or additional types of other features are not precluded. It will also be appreciated that specific embodiments have been described herein for purposes of illustration, but that various modifications may be made without deviating from the technology. Further, while advantages associated with some embodiments of the technology have been described in the context of those embodiments, other embodiments may also exhibit such advantages, and not all embodiments need necessarily exhibit such advantages to fall within the scope of the technology. Accordingly, the disclosure and associated technology can encompass other embodiments not expressly shown or described herein.

I/We claim:

1. A method of detecting blood flow and generating a vessel contour trace from an intravascular ultrasound (“IVUS”) image of a vessel, the method comprising:
receiving a plurality of frames of radiofrequency (RF) line data for the IVUS image;
generating an interim vessel contour trace of the vessel based on one or more of the frames of RF line data;
generating one or more user-interactable key nodes along the interim vessel contour trace;
generating a color mask representative of blood flow through the vessel based on one or more of the frames of RF line data; and
displaying a composite image on an output display, wherein the composite image comprises (a) the IVUS image, (b) the interim vessel trace and the user-interactable key nodes overlaid over the IVUS image, and (c) the color mask representative of blood flow through the vessel overlaid over the IVUS image.

2. The method of claim 1, further comprising:
modifying the interim vessel contour trace in response to user interaction with the one or more user-interactable key nodes.

3. The method of claim 1, wherein generating the interim vessel contour trace comprises:
generating a segmentation of the IVUS image to identify the vessel;
generating an initial vessel contour trace from the segmentation; and
refining the initial vessel contour trace using an active contour model constrained with a statistical vein shape model.

4. The method of claim 1, wherein generating the color mask representative of blood flow comprises:
selecting a first subframe from a first frame of the plurality of frames of RF line data;
selecting a second subframe from a second frame of the plurality of frames of RF line data;
performing block matching between the first subframe and the second subframe to generate similarity scores; and
converting the similarity scores to Cartesian coordinates to provide the color mask.

5. The method of claim 1, further comprising:
removing a ringdown artifact from one or more of the plurality of frames of RF line data prior to generating the interim vessel contour trace or the color mask.

6. The method of claim 1, further comprising:
applying one or more thresholds to the plurality of frames of RF line data; and
performing clutter filtering on the plurality of frames of RF line data based on the one or more thresholds.

7. The method of claim 1, further comprising:
generating and displaying on the output display one or more selected biometrics.

8. A method of generating a vessel contour trace from an intravascular ultrasound (“IVUS”) image, the method comprising:
receiving a first frame of RF line data for the IVUS image;
converting the first frame of RF line data to Cartesian coordinates and displaying the IVUS image;
receiving a user selection for the generation of the vessel contour trace;
generating a segmentation of the IVUS image to identify a vessel of interest for the vessel contour trace;
generating an initial vessel contour trace from the generated segmentation;
generating an interim vessel contour trace using an active contour model initialized with the initial vessel contour trace and constrained with a statistical vein shape model; and
displaying on an image output display, when the interim vessel contour trace meets or exceeds accuracy criteria, a complete vessel contour trace comprising the interim vessel contour trace with user-interactable key nodes or control points.

9. The method of claim 8, further comprising:
generating and displaying on the image output display one or more selected biometrics.

10. The method of claim 8, further comprising:
removing any ringdown artifact from the IVUS image.

11. The method of claim 8, wherein receiving a user selection for the generation of the vessel contour trace further comprises:
using a user-provided manual trace as the initial vessel contour trace and displaying the user-provided manual trace on the image output display when a user has selected autocompletion.

12. The method of claim 8, wherein receiving a user selection for the generation of the vessel contour trace further comprises:
using user-provided key nodes as accurate or true key nodes in the complete vessel contour trace when a user has selected autocompletion.

13. The method of claim 8, further comprising:
converting the initial vessel contour trace, from RF line data, into a Cartesian space coordinate system.

14. The method of claim 8, further comprising:
modifying the vessel contour trace in a region between an IVUS catheter and a vessel wall.

15. A method of detecting blood flow from intravascular ultrasound (“IVUS”) and generating a blood flow IVUS image, the method comprising:
receiving a plurality of frames of RF line data for the blood flow IVUS image, the plurality of frames of RF line data comprising a first frame of RF line data and a second frame of RF line data;
selecting a first subframe of a plurality of first subframes of the first RF line data frame;
selecting a second subframe of a plurality of second subframes of the second RF line data frame for search and comparison, the second subframe larger than the first subframe;
performing block matching for each selected first subframe, of the plurality of first subframes, with each corresponding portion of the selected second subframe, of the plurality of second subframes, and generating a similarity score for each selected comparison;
generating a third RF line data frame as a matrix of the plurality of similarity scores using a plurality of similarity scores from the block matching;
converting the matrix of the plurality of similarity scores to Cartesian coordinates to provide a color image or mask to display as a visual image of the blood flow;
generating a brightness-mode IVUS image from one or more frames of RF line data of the plurality of frames of RF line data;
merging or superimposing the color image or mask on or with the brightness-mode IVUS image to generate the blood flow IVUS image; and
displaying the blood flow IVUS image on an image output display.

16. The method of claim 15, wherein the block matching comprises a comparison of at least one first pattern of a plurality of speckle pixels within the selected first subframe with at least one second pattern of a plurality of speckle pixels within the selected second subframe.

17. The method of claim 15, further comprising:
receiving a user selection of or determining one or more signal acquisition modes.

18. The method of claim 15, further comprising:
receiving a user selection of blood flow detection and color parameters; and
applying the user selection of blood flow detection and color parameters.

19. The method of claim 15, further comprising:
performing clutter filtering of the first and second RF line data frames.

20. The method of claim 15, further comprising:
applying one or more thresholds to RF line data of the first or second frames of RF line data.
